(19) United States
(12) Patent Application Publication
(10) Pub. No.: US 2024/0174756 A1
(43) Pub. Date: May 30, 2024

(54) ANTIBODIES THAT BIND EGFR AND CMET

(71) Applicant: MERUS N.V., Utrecht (NL)

(72) Inventors: Cecilia Anna Wilhelmina GEUIJEN, Utrecht (NL); Robertus Cornelis ROOVERS, Utrecht (NL); Mark THROSBY, Utrecht (NL); Cornelis Adriaan DE KRUIF, Utrecht (NL); Ton LOGTENBERG, Utrecht (NL)

(21) Appl. No.: 18/446,795

(22) Filed: Aug. 9, 2023

Related U.S. Application Data
(62) Division of application No. 16/637,464, filed on Feb. 7, 2020, now Pat. No. 11,773,170.

(30) Foreign Application Priority Data
Aug. 9, 2017 (EP) 17185572.9

(51) Int. Cl. C07K 16/28; A61P 35/00; C07K 16/40

(52) U.S. Cl. CPC C07K 16/2863; A61P 35/00; C07K 16/40; A61K 2039/505

(57) ABSTRACT

The invention as disclosed herein relates to bispecific antibodies that comprises a first variable domain that can bind an extracellular part of epidermal growth factor receptor (EGFR) and a second variable domain that can bind an extracellular part of MET Proto-Oncogene, Receptor Tyrosine Kinase (cMET). The antibody may comprise a common light chain. It may be a human antibody. The antibody may be a full-length antibody. In some aspects, the bispecific antibody is an IgG1 format antibody having an anti-EGFR, anti-cMET stoichiometry of 1:1. In some aspects, the antibody has one variable domain that can bind EGFR and one variable domain that can bind cMET.

### CROSS REFERENCE TO RELATED APPLICATIONS

This application is a division of U.S. application Ser. No. 16/637/464 filed Feb. 7, 2020, now U.S. Pat. No. 11,773,170, which is a U.S. national stage filing of International Application No. PCT/NL2018/050537, filed Aug. 9, 2018; which claims priority to EP Application No. 17185572.9, filed Aug. 9, 2017. The entire contents of International Application No. PCT/NL2018/050537 are hereby incorporated herein by reference.

### REFERENCE TO A SEQUENCE LISTING SUBMITTED ELECTRONICALLY VIA EFS-WEB

This application includes a Sequence Listing submitted electronically via EFS-Web (Name: 4096_0310002_Seqlisting_ST26; Size 240,560 bytes; and Date of Creation: Aug. 7, 2023), which is hereby incorporated by reference in its entirety.

The invention relates to the field of antibodies. In particular it relates to the field of therapeutic antibodies, including human antibodies, for the treatment of diseases involving aberrant cells. Further, it relates to antibodies that bind EGFR and cMET, including multispecific antibodies, and their use in the binding of EGFR and cMET positive cells, particularly tumor cells.

The epidermal growth factor (EGF) receptor (EGFR) is a cell-surface receptor for members of the epidermal growth factor family (EGF-family) of extracellular protein ligands. EGFR is also known as the ErbB-1 receptor. The receptor has been given various names in the past (EGFR; ERBB; ERBB1; HER1; PIG61; mENA). In the present invention the names ErbB-1, EGFR or HER1 in humans will be used interchangeably. EGFR is a member of the ErbB family of receptors, a subfamily of four closely related receptor tyrosine kinases: ErbB-1 (EGFR), ErbB-2 (HER2/c-neu; Her2), ErbB-3 (Her 3) and ErbB-4 (Her 4).

EGFR exists on a cell surface and may be activated by binding of its specific ligands, including epidermal growth factor and transforming growth factor α (TGFα). Upon activation by its growth factor ligands, the receptor may undergo a transition from an inactive mostly monomeric form to an active homodimer. In addition to forming homodimers after ligand binding, EGFR may pair with another member of the ErbB receptor family, such as ErbB2, to create an activated heterodimer. Dimers may also form in the absence of ligand-binding and clusters of activated EGFRs may form after ligand binding.

EGFR dimerization stimulates intrinsic intracellular protein-tyrosine kinase (PTK) activity. This activity induces several signal transduction cascades that lead to cell proliferation and differentiation. The kinase domain of EGFR can cross-phosphorylate tyrosine residues of other receptors it is complexed with, and can itself be activated in that manner.

Mutations involving EGFR have been identified in several types of cancer. It is the target of an expanding class of anticancer therapies. Such therapies include EGFR tyrosine kinase inhibitors (EGFR-TKIs) such as gefitinib and erlotinib for lung cancer, and antibodies as cetuximab and panitumumab for colon cancer and head and neck cancer.

Cetuximab and panitumumab are monoclonal antibodies that inhibit the receptor. Other monoclonals in clinical development are zalutumumab, nimotuzumab, and matuzumab. The monoclonal antibodies aim to block the extracellular ligand-induced receptor activation, mostly by blocking ligand binding to the receptor. With the binding site blocked, signal-inducing molecules may not attach effectively and thereby also not activate downstream signaling. Ligand-induced receptor activation may also be inhibited by stabilization of the inactive receptor conformation (matuzumab).

To date, EGFR targeted therapies have been associated with the development of treatment resistance over time. Various mechanisms for the resistance to EGFR-TKIs have been described. In patients with advanced non-small cell lung cancer (NSCLC) the mechanisms of resistance include the occurrence of secondary mutations (e.g., T790M, C7975), the activation of alternative signaling (e.g., Met, HGF, AXL, Hh, IGF-1R), aberrant downstream pathways (e.g., AKT mutations, loss of PTEN), the impairment of the EGFR-TKIs-mediated apoptosis pathway (e.g., BCL2-like 11/BIM deletion polymorphism) and histological transformation. Although some mechanisms of resistance have been identified others remain to be identified. Similarly, patients with colorectal cancer that are treated with EGFR antibodies also develop resistance over time. This may occur through emergence of KRAS mutations. Of those without KRAS mutations; amplification of the MET proto-oncogene may be associated with acquired resistance during anti-EGFR therapy (Bardelli et al., 2013; Cancer Discov. Jun;3(6):658-73. doi: 10.1158/2159-8290.CD-12-0558). The tumor can be resistant ab initio or develop resistance during treatment. Resistance to EGFR-targeted therapy is seen in many EGFR positive cancers and has demonstrated a need in the art for more efficacious EGFR cancer treatments that improve the standard of care, and are superior in terms of the capacity to address EGFR-targeted therapy resistance.

Dysregulation of MET Proto-Oncogene, Receptor Tyrosine Kinase (cMET) and hepatocyte growth factor (HGF) have been reported in a variety of tumors. Ligand-driven cMET activation has been observed in several cancers. Elevated serum and intra-tumoral HGF is observed in lung, breast cancer, and multiple myeloma (J. M. Siegfried et al., Ann Thorac Surg 66, 1915 (1998); P. C. Ma et al., Anticancer Res 23, 49 (2003); B. E. Elliott et al. Can J Physiol Pharmacol 80, 91 (2002); C. Seidel, et al, Med Oncol 15, 145 (1998)). Overexpression of cMET, cMET amplification or mutation has been reported in various cancers such as colorectal, lung, gastric, and kidney cancer and may drive ligand-independent receptor activation (C. Birchmeier et al, Nat Rev Mol Cell Biol 4, 915 (2003); G. Maulik et al., Cytokine Growth Factor Rev 13, 41 (2002)). Expression of HGF is also associated with the activation of the HGF/cMET signaling pathway and is also one of the escape mechanisms of tumors under selection by EGFR targeted therapy.

The cMET receptor is formed by proteolytic processing of a common precursor into a single-pass, disulphide-linked α/β heterodimer. The extracellular portion of cMET is composed of three domain types. The N-terminal region fold forms a large semaphorin (Sema) domain, which encompasses the whole a-subunit and part of the β-subunit. The plexin-semaphorin-integrin (PSI) domain follows the Sema domain, and includes four disulphide bonds. This domain is connected to the transmembrane helix via four immunoglobulin-plexin-transcription (IPT) domains, which are related to immunoglobulin-like domains. Intracellularly, the cMET receptor contains a tyrosine kinase catalytic domain flanked by distinctive juxtamembrane and carboxy-terminal sequences (Organ and Tsao. Therapeutic advances in medical oncology 3.1_suppl (2011): S7-S19 which is incorporated herein by reference in its entirety).

The ligand of cMET, hepatocyte growth factor (HGF; also known as scatter factor) and its splicing isoforms (NK1, NK2) are known ligands of the cMET receptor.

HGF was identified in 1991 as a potent mitogen/morphogen. The HGF/cMET signaling pathway plays important roles in the development and progression of various cancers. Dysregulation and/or hyperactivation of HGF or cMET in human cancers are linked to poor prognosis. cMET can be activated via overexpression, amplification, or mutation. Activation may promote development, progression, invasive growth, and metastasis of cancers. cMET can be activated in an HGF associated and HGF independent fashion. HGF independent activation occurs in cases of cMET over-expression. Abundance of cMET also may trigger (hetero)dimerization and intra-cellular signaling in the absence of ligand. Additional ligand does not appear to affect the function of such cMET over-expression cells. cMET amplification is associated with cMET over-expression and has emerged as a biomarker of tumor subtypes.

HGF is expressed ubiquitously throughout the body, showing this growth factor to be a systemically available cytokine as well as coming from the tumor stroma. A positive paracrine and/or autocrine loop of cMET activation can lead to further cMET expression. The HGF specific antibody Rilotumumab (AMG102) was developed for gastric cancer. Phase I and Phase II trials appeared promising but a phase III study with cisplatin and capecitabine as a first-line therapy in gastric cancer (RILOMET-2) was terminated following a pre-planned data monitoring committee safety review of study 20070622.

The relevance of cMET/HGF signaling in resistance to EGFR-targeted therapies has stimulated the development of ways to deal with the resistance. To date, antibody based approaches include anti-HGF antibodies; anti cMET or cMET antibodies and cMET/EGFR (reviewed in Lee et al., 2015; Immunotargets and Therapy 4: 35-44) have not been clinically effective. The cMET antibodies Onartuzumab (MetMab™) and Emibetuzumab (LY-2875358) have been evaluated in phase II clinical trials. Of these Onartuzumab appeared to be effective against colorectal cancer in a combination treatment together with the EGFR-inhibitor erlotinib. These results could, however, not be repeated in a randomized phase III clinical trial. MetMAb is a monovalent monoclonal antibody (mAb) against cMET, which blocks HGF binding to cMET and subsequent pathway activation (Jin et al., 2008 Cancer Research Vol. 68: pp 4360-68).

Overcoming a problem with anti-EGFR, cMET and HGF immunotherapies, the present invention provides novel bispecific antibodies that comprise a first variable domain that can bind an extracellular part of epidermal growth factor receptor (EGFR) and a second variable domain that can bind an extracellular part of cMET Proto-Oncogene, Receptor Tyrosine Kinase (cMET).

To date, certain bispecific EGFR x cMET antibodies have been described in the art. Castoldi R. et al. (2013) describe a bispecific EGFR x cMET antibody designated MetHer1 with the cMET binding site of the antibody 5D5 (or MetMab) and the EGFR binding site of cetuximab. The bispecific antibody has a fixed EGFR and cMET binding stoichiometry of 2:1 (see Supplemental Figures)

US20140378664 describes a cMET x EGFR bispecific antibody among various other bispecific antibodies. The complete bispecific antibody is produced as a single protein which is later proteolytically cleaved. The two VH/VL domains are produced as single chain Fv fragments. Binding of the antibody induces cMET degradation and Akt phosphorylation in a gastric cancer cell line. Moores et al (2016) describe a bispecific cMET x EGFR antibody designated JNJ-61186372 produced by controlled Fab-arm exchange (cFAE) having mutations at position 405 and 409 according to EU numbering, which may have potential for immunogenicity. JNJ-61186372 was shown to be active in vivo using a xenograft model with tumor cell line H1975 that expresses the cMET ligand HGF. This tumor model is known to be dependent on the ADCC activity of the antibody (Ahmed et al., 2015). JNJ-61186372 has a reported affinity imbalance of approximately 40×greater affinity for cMET than EGFR (Moores et al. (2016)), and the anti-EGFR arm derived from zalutumumab is known to cause infusion related reaction, skin disorders, among other issues.

LY3164530 is a bispecific cMET x EGFR antibody, which contains the EGFR binding domain of cetuximab as a single chain Fv fragment fused to the heavy chain variable domain of the cMET binding antibody LY2875358 (Emibetuzumab; Kim and Kim 2017). It is a so-called dual variable domain antibody that comprises two binding sites for each of the antigens. No data are provided on HGF inhibition for the antibody. The antibody reportedly binds and internalizes cMET and EGFR without agonistic activity. The authors review various cMET, EGFR and cMET x EGFR targeted therapies and draw the conclusion that to date none of these inhibitors have shown significant efficacy in clinical trials.

There is thus a need for novel bispecific cMET x EGFR antibodies, including those which may have superior characteristics as described herein.

### SUMMARY OF THE INVENTION

In one aspect the invention provides a bispecific antibody that comprises a first variable domain that can bind an extracellular part of human epidermal growth factor receptor (EGFR) and a second variable domain that can bind an extracellular part of human MET Proto-Oncogene, Receptor Tyrosine Kinase (cMET).

The bispecific antibody may comprise a common light chain. The first and second variable domains preferably comprise the same or substantially the same (common) light chain variable region. Said common light chain variable region may be one that is known to pair well with a diversity of human variable region gene segments that have undergone recombination. More preferably said common light chain is preferably a variable region encoded by a germline Vk gene segment, preferably the O12/IgV_{K}1-39*01 variable region gene segment. The preferred light chain variable region comprises the rearranged IgV_{K}1-39*01/IGJ_{K}1*01 or IgV_{K}1-39*01/IGJ_{K}5*01. The light chain of the cMET binding arm and the light chain of the EGFR binding arm is preferably the same (common) light chain. The common light chain is preferably the rearranged kappa light chain IgV_{K}1-39*01/IGJ_{K}1*01 or IgV_{K}1-39*01/IGJ_{K}5*01 joined to a human light chain constant region. The bispecific antibody can be a human antibody. The bispecific antibody can be a full length antibody. It may have one variable domain that can bind EGFR and one variable domain that can bind cMET. In one aspect the variable domain that can bind human EGFR can also beneficially bind mouse EGFR and/or cynomolgus EGFR. The variable domain that can bind human EGFR may bind to domain III of human EGFR. The variable domain that can bind cMET may block the binding of antibody 5D5 to cMET. The variable domain that can bind cMET may block the binding of HGF to cMET. The Kd of the antibody for cMET can be at least 10 times less than the Kd of the antibody for EGFR. The amino acids at positions 405 and 409 in one CH3 domain may be the same as the amino acids at the corresponding positions in the other CH3 domain (EU-numbering).

The first variable domain may comprise a heavy chain variable region with a CDR1 sequence SYGIS (SEQ ID NO: 24); a CDR2 sequence WISAYX₁X₂NTNYAQKLQG (SEQ ID NO: 25) and a CDR3 comprising the sequence X₃X₄X₅X₆HWWLX₇A (SEQ ID NO: 26) wherein X₁=N or S; X₂=A or G; X₃=D or G; X₄=R, S or Y; X₅=H, L or Y; X₆=D or W and X₇=D or G; with 0-5 amino acid insertions, deletions, substitutions, additions or a combination thereof at a position other than X₁-X₇.

The second variable domain may comprise a heavy chain variable region with the amino acid sequence of one of the sequences of SEQ ID NO: 1-23 with 0-10 preferably 0-5 amino acid insertions, deletions, substitutions, additions or a combination thereof.

Bispecific antibodies are described wherein
X₁ = N; X₂ = G; X₃ = D; X₄ = S; X₅ = Y; X₆ = W and
X₇ = G;
(SEQ ID NO: 113)
WISAYNGNTNYAQKLQG
and
(SEQ ID NO: 153)
DSYWHWWLGA,
X₁ = N; X₂ = A; X₃ = D; X₄ = S; X₅ = Y; X₆ = W and
X₇ = G;
(SEQ ID NO: 25)
WISAYNANTNYAQKLQG
and
(SEQ ID NO: 153)
DSYWHWWLGA,
X₁ = S; X₂ = G; X₃ = D; X₄ = S; X₅ = Y; X₆ = W and
X₇ = G;
(SEQ ID NO: 140)
WISAYSGNTNYAQKLQG
and
(SEQ ID NO: 153)
DSYWHWWLGA,
X₁ = N; X₂ = G; X₃ = D; X₄ = R; X₅ = H; X₆ = W and
X₇ = D;
(SEQ ID NO: 113)
WISAYNGNTNYAQKLQG
and
(SEQ ID NO: 139)
DRHWHWWLDA,
X₁ = N; X₂ = A; X₃ = D; X₄ = R; X₅ = H; X₆ = W and
X₇ = D;
(SEQ ID NO: 25)
WISAYNANTNYAQKLQG
and
(SEQ ID NO: 139)
DRHWHWWLDA,
X₁ = S; X₂ = G; X₃ = D; X₄ = R; X₅ = H; X₆ = W and
X₇ = D;
(SEQ ID NO: 140)
WISAYSGNTNYAQKLQG
and
(SEQ ID NO: 139)
DRHWHWWLDA,
X₁ = N; X₂ = G; X₃ = G; X₄ = Y; X₅ = L; X₆ = D and
X₇ = G;
(SEQ ID NO: 113)
WISAYNGNTNYAQKLQG
and
(SEQ ID NO: 154)
GYLDHWWLGA,
X₁ = N; X₂ = A; X₃ = G; X₄ = Y; X₅ = L; X₆ = D and
X₇ = G;
(SEQ ID NO: 25)
WISAYNANTNYAQKLQG
and
(SEQ ID NO: 154)
GYLDHWWLGA,
or
X₁ = S; X₂ = G; X₃ = G; X₄ = Y; X₅ = L; X₆ = D and
X₇ = G,
(SEQ ID NO: 140)
WISAYSGNTNYAQKLQG
and
(SEQ ID NO: 154)
GYLDHWWLGA.
In some embodiments
X₁ = N; X₂ = G; X₃ = D; X₄ = R; X₅ = H; X₆ = W and
X₇ = D;
(SEQ ID NO: 113)
WISAYNGNTNYAQKLQG
and
(SEQ ID NO: 139)
DRHWHWWLDA,
X₁ = N; X₂ = A; X₃ = D; X₄ = R; X₅ = H; X₆ = W and
X₇ = D;
(SEQ ID NO: 25)
WISAYNANTNYAQKLQG
and
(SEQ ID NO: 139)
DRHWHWWLDA,
or
X₁ = S; X₂ = G; X₃ = D; X₄ = R; X₅ = H; X₆ = W and
X₇ = D
(SEQ ID NO: 140)
WISAYSGNTNYAQKLQG
and
(SEQ ID NO: 139)
DRHWHWWLDA.

In a preferred embodiment X₃-X_{7 =}DRHWD (SEQ ID NO: 27) and X₁ and X₂ are NG; SG or NA.

Bispecific antibodies are described wherein the heavy chain variable region of the second variable domain comprises the amino acid sequence of one of the sequences of SEQ ID NO: 1-3; 7; 8; 10; 13; 15; 16; 17; 21; 22 or 23 with 0-10 preferably 0-5 amino acid insertions, deletions, substitutions, additions or a combination thereof.

The invention also provides a method of treatment of a subject that has a tumor the method comprising administering the bispecific antibody as described herein to the individual in need thereof. Typically, the individual is one suffering from a disease involving aberrant cells, for examples the individual may be suffering from a tumor.

The invention also provides a bispecific antibody that comprises a first variable domain that can bind an extracellular part of epidermal growth factor receptor (EGFR) and a second variable domain that can bind an extracellular part of MET Proto-Oncogene, Receptor Tyrosine Kinase (cMET), wherein the first variable domain comprises a heavy chain variable region with a CDR1 sequence SYGIS (SEQ ID NO: 24); a CDR2 sequence WISAYX₁X₂NTNYAQKLQG (SEQ ID NO: 25) and a CDR3 comprising the sequence X₃X₄X₅X₆HWWLX₇A (SEQ ID NO: 26) wherein

X₁=N or S; X₂=A or G; X₃=D or G; X₄=R, S or Y; X₅=H, L or Y; X₆=D or W and X₇=D or G with 0-5 amino acid insertions, deletions, substitutions, additions or a combination thereof at a position other than X₁-X₇ and wherein the second variable domain comprises a heavy chain variable region with the amino acid sequence of one of the sequences of SEQ ID NO: 1-23 with 0-10 preferably 0-5 amino acid insertions, deletions, substitutions, additions or a combination thereof.

The first variable domain preferably comprises a heavy chain variable region with a CDR1 sequence SYGIS (SEQ ID NO: 24); a CDR2 sequence WISAYNGNTNYAQKLQG (SEQ ID NO: 113) and a CDR3 comprising the sequence DRHWHWWLDAFDY (SEQ ID NO: 37) and the second variable domain preferably comprises a heavy chain variable region with a CDR1 sequence SYSMN (SEQ ID NO: 28); a CDR2 sequence WINTYTGDPTYAQGFTG (SEQ ID NO: 29) and a CDR3 sequence ETYYYDRGGYPFDP (SEQ ID NO: 30).

The invention also provides a bispecific antibody of an invention as disclosed herein for use in the treatment of a subject that has a disease involving abserrant cells, such as a tumor.

Also provided is a use of a bispecific antibody of an invention as disclosed herein in the manufacture of a medicament for the treatment of a disease involving aberrant cells, such as a tumor or cancer.

Also provided is a method of treatment of a subject that has a tumor, preferably an EGFR positive tumor, a cMET positive tumor or an EGFR and cMET positive tumor, the method comprising administering the bispecific antibody to the individual in need thereof.

An antibody of an invention as disclosed herein preferably inhibits HGF induced migration of EBC1 cells in a wound healing assay. Preferably the inhibition is better than the combination of cetuximab and MetMab. For example, it is preferred to achieve inhibition via prevention of wound closure in the presence of HGF with or without EGF (HGF is present at 15 ng/ml and EGF, when present, is present in amount of 12.5 ng/ml).

An antibody of an invention disclosed herein inhibits HGF and EGF/HGF induced growth of the EGFR TKI resistant tumor cell lines PC-9 and HCC827 when used in combination with a TKI. The TKI is preferably gefitinib.

An antibody of an invention disclosed herein inhibits HGF induced growth of an HGF responsive cell, preferably of the EGFR TKI resistant tumor cell line PC-9 or HCC827.

An antibody of an invention disclosed herein inhibits EGF induced growth of an EGF responsive cell, without inducing the toxicities such as rash and diarrhea associated with high affinity bivalent EGFR antibodies. This renders the antibody ideally suited for combination with TKI which have its own toxicity profile.

The invention further comprises a pharmaceutical composition that comprises a bispecific antibody disclosed herein.

An antibody of the invention may be used to treat a tumor which is resistant to treatment with an EGFR tyrosine kinase inhibitor, for example resistant to erlotinib, gefitinib, or afatinib, an analogue of erlotinib, gefitinib or afatinib or a combination of one or more of the respective compounds and/or analogues thereof.

Treatment according to the invention may further comprise treatment with an EGFR tyrosine kinase inhibitor for example wherein the EGFR tyrosine kinase inhibitor is erlotinib.

Accordingly, the bispecific antibody of the invention may be administered simultaneously, sequentially or separately with an EGFR tyrosine kinase inhibitor.

The invention further comprises a nucleic acid molecule or a group of nucleic acid molecules that alone or together encode a heavy chain(s) or a heavy chain variable region(s) of a bispecific antibody disclosed herein or a variant thereof. Also provided is a nucleic acid molecule or group of nucleic acid molecules that encode an antibody disclosed herein.

In a preferred embodiment the heavy chain comprises a constant region of an IgG1 antibody, preferably a human IgG1 antibody. The CH2 region of said IgG1 constant region can be engineered to alter ADCC and/or CDC activity of the antibody, or not. In a preferred embodiment, said alteration results in enhanced ADCC and/or CDC activity. In a preferred embodiment the CH3-region of the antibody is engineered to facilitate heterodimerization of heavy chains comprising a first heavy chain that binds EGFR and a second heavy chain binds cMET.

The invention further comprises is a cell comprising one or more nucleic acid molecules that alone or together encode a bispecific antibody or a variant thereof as disclosed herein. Also provided are methods of producing a bispecific antibody or a variant thereof disclosed herein using a cell as described, preferably together with the harvesting of the bispecific antibody or variant thereof from a culture of the cells.

The invention further comprises a cell system that comprises a bispecific antibody or variant thereof disclosed herein.

The invention further provides a cell that expresses the bispecific antibody and/or comprises the nucleic acid molecule(s) that encode said bispecific antibody.

The invention further comprises a bispecific antibody as disclosed herein that further comprises a label, preferably a label for in vivo imaging.

### DETAILED DESCRIPTION OF THE INVENTION

EGFR is a member of a family of four receptor tyrosine kinases (RTKs), named Her- or cErbB-1, -2, -3 and -4. The EGFR has an extracellular domain (ECD) that is composed of four sub-domains, two of which are involved in ligand binding and one of which is involved in homo-dimerization and hetero-dimerization Ferguson(2008). The reference numbers used in this section refer to the numbering of the references in the list headed “cited in the specification”, which are each incorporated by reference. EGFR integrates extracellular signals from a variety of ligands to yield diverse intracellular responses (Yarden at al. 2001; and Jorrisen et al. 2003). The EGFR is implicated in several human epithelial malignancies, notably cancers of the breast, bladder, non-small cell lung cancer lung, colon, ovarian head and neck and brain. Activating mutations in the gene have been found, as well as over-expression of the receptor and of its ligands, giving rise to autocrine activation loops (for review, see Robertson et al. 2000). This RTK has therefore been extensively used as target for cancer therapy. Both small-molecule inhibitors targeting the RTK and monoclonal antibodies (mAbs) directed to the extracellular ligand-binding domains have been developed and have shown hitherto several clinical successes, albeit mostly for a select group of patients. Database accession numbers for the human EGFR protein and the gene encoding it are (GenBank NM_005228.3). Other database identifiers for the gene and/or protein are HGNC: 3236; Entrez Gene: 1956; Ensembl: ENSG00000146648; OMIM: 131550 and UniProtKB: P00533. The accession numbers are primarily given to provide a further method of identification of EGFR protein as a target, the actual sequence of the EGFR protein bound by an antibody may vary, for instance because of a mutation in the encoding gene such as those occurring in some cancers or the like. Where reference herein is made to

EGFR, the reference refers to human EGFR unless otherwise stated. The antigen-binding site that binds EGFR, binds EGFR and a variety of variants thereof such as those expressed on some EGFR positive tumors.

The term “EGFR ligand” as used herein refers to polypeptides which bind and activate EGFR. Examples of EGFR ligands include, but are not limited to EGF, TGF-α, HB-EGF, amphiregulin, betacellulin and epiregulin (for review Olayioye MA et al.; EMBO J (2000) Vol 19: pp 3159-3167). The term includes biologically active fragments and/or variants of a naturally occurring polypeptide

cMET, also called tyrosine-protein kinase MET or hepatocyte growth factor receptor (HGFR), is a protein that in humans is encoded by the MET gene. The protein possesses tyrosine kinase activity. The primary single chain precursor protein is post-translationally cleaved to produce the alpha and beta subunits, which are disulfide linked to form the mature receptor.

Aberrantly activated cMET may induce tumor growth, the formation of new blood vessels (angiogenesis) that supply the tumor with nutrients, and cancer spread to other organs (metastasis). cMET is deregulated in many types of human malignancies, including cancers of kidney, liver, stomach, breast, and brain. The cMET gene is known under a number of different names such as MET Proto-Oncogene, Receptor Tyrosine Kinase; Hepatocyte Growth Factor Receptor; Tyrosine-Protein Kinase Met; Scatter Factor Receptor; Proto-Oncogene C-Met; HGF/SF Receptor; HGF Receptor; SF Receptor; EC 2.7.10.1; Met Proto-Oncogene; EC 2.7.10; DFNB97; AUTS9; RCCP2; C-Met; MET; HGFR; External Ids for cMET are HGNC: 7029; Entrez Gene: 4233; Ensembl: ENSG00000105976; OMIM: 164860 and UniProtKB: P08581. The accession numbers are primarily given to provide a further method of identification of cMET protein as a target, the actual sequence of the cMET protein bound by an antibody may vary, for instance because of a mutation in the encoding gene such as those occurring in some cancers or the like. Where reference herein is made to cMET, the reference refers to human cMET unless otherwise stated. The antigen-binding site that binds cMET, binds cMET and a variety of variants thereof such as those expressed on some cMET positive tumors.

An antibody typically recognizes only a part of an antigen. The antigen is typically but not necessarily a protein. The recognition or binding site on an antigen, bound by an antibody is referred to as the epitope, where an epitope may be linear or conformational. Binding of an antibody to an antigen is typically specific. The ‘specificity’ of an antibody refers to its selectivity for a particular epitope, whereas ‘affinity’ refers to the strength of the interaction between the antibody's antigen binding site and the epitope it binds.

Exemplary antibodies of an invention disclosed herein binds to EGFR and cMET, preferably human EGFR and human cMET. An EGFR/cMET bispecific antibody of an invention disclosed herein binds to EGFR and, under otherwise identical conditions, at least 100-fold less to the homologous receptors ErbB-2 and ErbB-4 of the same species. An EGFR/cMET bispecific antibody of an invention as disclosed herein binds to cMET and, under otherwise identical conditions, at least 100-fold less to the receptors ErbB-2 and ErbB-4 of the same species. Considering that the receptors are cell surface receptors, the binding may be assessed on cells that express the receptor(s). A bispecific antibody of an invention disclosed herein preferably binds to human, cynomolgus EGFR and/or to mouse EGFR.

An antibody that binds EGFR and cMET may bind other proteins as well if such other proteins contain the same epitope. Hence, the term “binding” does not exclude binding of the antibodies to another protein or protein(s) that contain the same epitope. Such binding is typically referred to as cross-reactivity. An EGFR/cMET bispecific antibody typically does not bind to other proteins than EGFR and/or cMET on the membrane of cells in a post-natal, preferably adult human. An antibody according to an invention disclosed herein is typically capable of binding EGFR with a binding affinity (i.e. equilibrium dissociation constant Kd) of at least 1×10e−6 M, as outlined in more detail below.

The term “antibody” as used herein means a proteinaceous molecule preferably belonging to the immunoglobulin class of proteins. An antibody typically contains two variable domains that bind an epitope on an antigen. Such domains are derived from or share sequence homology with the variable domain of an antibody. A bispecific antibody of an invention as disclosed herein preferably comprises two variable domains. Antibodies for therapeutic use are preferably as close to natural antibodies of the subject to be treated as possible (for instance human antibodies for human subjects). Antibody binding can be expressed in terms of specificity and affinity The specificity determines which antigen or epitope thereof is specifically bound by the binding domain. Typically, antibodies for therapeutic applications can have affinities of up to 1×10e−10 M or higher. Antibodies such as bispecific antibodies of an invention disclosed herein preferably comprise the constant domains (Fc part) of a natural antibody. An antibody of invention as disclosed herein is typically a bispecific full length antibody, preferably of the human IgG subclass. Preferably, the antibodies of the present invention are of the human IgG1 subclass. Such antibodies of invention as disclosed herein can have good ADCC properties, have a favorable half-life upon in vivo administration to humans and CH3 engineering technology exists that can provide for modified heavy chains that preferentially form hetero-dimers over homo-dimers upon co-expression in clonal cells. ADCC activity of an antibody can also be improved through techniques known to persons of skill in the art.

An antibody of an invention as disclosed herein is preferably a “full length” antibody. The term ‘full length’ according to an invention as disclosed herein is defined as comprising an essentially complete antibody, which however does not necessarily have all functions of an intact antibody. For the avoidance of doubt, a full length antibody contains two heavy and two light chains. Each chain contains constant (C) and variable (V) regions, which can be broken down into domains designated CH1, CH2, CH3, VH, and CL, VL. Typically, an antibody binds to antigen via the variable domains contained in the Fab portion, and after binding can interact with molecules and cells of the immune system through the constant domains, mostly through the Fc portion. Full length antibodies according to an invention disclosed herein encompasses antibodies wherein mutations may be present that provide desired characteristics. Antibodies wherein one or several amino acid residues are deleted, without essentially altering the specificity and/or affinity characteristics of the resulting antibody are embraced within the term “full length antibody”. For instance, an IgG antibody can have 1-20 amino acid residue insertions, deletions, or substitutions or a combination thereof in the constant region.

An antibody of an invention as disclosed herein is preferably a bispecific IgG antibody, preferably a bispecific full length IgG1 antibody and more preferably a human IgG1. Full length IgG antibodies are preferred because of their typically favorable half-life and the desire to stay as close to fully autologous (human) molecules for reasons of immunogenicity. In some embodiments, an antibody of the invention is a full length IgG1, a full length IgG2, a full length IgG3 or a full length IgG4 antibody.

An invention disclosed herein includes a bispecific antibody that comprises a first variable domain that can bind an extracellular part of EGFR and a second variable domain that can bind an extracellular part of cMET wherein the first variable domain binds EGFR with an affinity that is less than cetuximab which has a Kd of 0.39 nM (Kim et al 2008). The first variable domain preferably binds EGFR with a Kd that is between 10e−6 M and 10e−9 M. The Kd is preferably between 10e−7 M and 10e−9 M, preferably between 10e−8 M and 10e−9 M. The second variable domain preferably binds cMET with a Kd that is 10e−7 M or less. The Kd is preferably between 10e−7 M and 10e−11 M. The second variable domain preferably has a higher affinity for cMET than the first variable domain has for EGFR. In other words in this preferred embodiment the Kd of the antibody for cMET is less than the Kd of the antibody for EGFR. In a preferred embodiment the Kd of the antibody for cMET is at least 5 and preferably at least 10×less than the Kd of the antibody for EGFR. In this embodiment the values for the Kd for the respective antigens are preferably as indicated in this paragraph. This appropriate imbalance of affinity permits the bispecific antibody of an invention disclosed herein to dock on a cell preferably via binding to EGFR and block the binding the ligand HGF to cMET.

The variable domain that can bind EGFR is preferably a variable domain that, in the context of a bivalent monospecific antibody, inhibits EGF induced death of A431 cells. Inhibition of EGF induced cell death is preferably measured at a concentration of 10 nM EGF and 10 μg/ml antibody. Inhibition of EGF induced cell death is detectable by comparing the number of cells with and without the antibody after a 3-7 of day of culture of the A431 under conditions that are permissive (but for the EGF) for A431cell growth. Without being bound by theory it is believed that the binding of the antibody to EGFR blocks the binding of EGF to EGFR. The variable domain that can bind EGFR is preferably a variable domain that, in the context of a bivalent monospecific antibody, inhibits EGF induced proliferation of BxPC3 or BxPC3-luc2 cells.

An antibody of an invention as disclosed herein preferably inhibits HGF induced migration of EBC1 cells in a wound healing assay. The wound healing assay is preferably an assay as described in the examples. The inhibition of wound healing is better than the combination of cetuximab and MetMab. The inhibition is typically not 100%. Some wound healing also occurs in the presence of an inhibitory antibody.

An antibody of an invention as disclosed herein inhibits HGF and EGF/HGF induced growth of the EGFR TKI resistant tumor cell lines PC-9 and HCC827 when used in combination with a TKI. The TKI is preferably gefitinib.

An antibody of an invention as disclosed herein inhibits HGF induced growth of an HGF responsive cell, preferably of the EGFR TKI resistant tumor cell line PC-9 or HCC827.

An antibody of an invention as disclosed herein inhibits EGF induced growth of an EGF responsive cell, without inducing significant common toxicities such as rash and diarrhea, etc. associated with high affinity bivalent EGFR antibodies. This renders the antibody ideally suited for combination with TKIs which have their own toxicity profile.

The induced growth is preferably measured using an assay as described in the examples. The inhibition is typically not 100%. Some growth occurs also in the context of an inhibitory antibody.

The variable domain that can bind EGFR and that comprises the amino acid sequence of the MF3370 or variant thereof as indicated herein, preferably binds to EGFR domain III (see table 4 of international patent application PCT/NL2015/050124; WO2015/130172 which is incorporated by reference herein). The binding of the variable domain to EGFR can be inhibited by cetuximab. The variable domain binds an epitope that is different from the epitope that is recognized by cetuximab and zalutumumab. For example, the variable domain binds to mouse EGFR whereas cetuximab and zalutumumab do not, indicating that one or more of the residues that differ between mouse and human EGFR domain III play a role in cetuximab and zalutumumab binding, but not in an antibody of an invention described herein. An advantage of a bispecific antibody of an invention described herein having human, mouse, cynomolgus EGFR cross-reactivity is that it permits the use of xenograft studies with human cancer models, which may be more predictive with respect to effectivity and toxicity as the antibody also binds to the normal mouse cells that have the receptor, while also being capable of use in cynomolgus toxicology studies. In one aspect the invention provides a bispecific antibody that comprises a first variable domain that can bind an extracellular part of human epidermal growth factor receptor (EGFR) and a second variable domain that can bind an extracellular part of human MET Proto-Oncogene, Receptor Tyrosine Kinase (cMET), wherein said first variable domain can also bind mouse EGFR, cynomolgus EGFR or both.

A cMET variable domain preferably comprises an amino acid sequence of the MF4356 or variant thereof as indicated herein, and preferably blocks the binding of the antibody MetMab to cMET. The variable domain preferably blocks the binding of the ligand HGF to cMET. The variable domain blocks the binding of the antibody MetMab to cMET when the binding of MetMab to cMET at half-maximum binding conditions is reduced by at least 40% and preferably at least 60% in the presence of a saturating amount of said variable domain. The variable domain is preferably provided in the context of a bivalent monospecific antibody. The cMET variable domain can preferably bind the sema domain of cMET. The cMET variable domain of the invention may compete with 5D5 for binding cMET or not compete with reported anti-cMET reference antibodies, such as 5D5. See Table 2.

A variable domain of an invention disclosed herein can bind EGFR (the first variable domain) and preferably comprises a heavy chain variable region with a CDR1 sequence SYGIS (SEQ ID NO: 24); a CDR2 sequence WISAYX₁X₂NTNYAQKLQG (SEQ ID NO: 25) and a CDR3 comprising the sequence X₃X₄X₅X₆HWWLX₇A (SEQ ID NO: 26) wherein X₁=N or S; X₂=A or G; X₃=D or G; X₄=R, S or Y; X₅=H, L or Y; X₆=D or W and X₇=D or G.

X₁₋₇ is preferably:
X₁ = N; X₂ = G; X₃ = D; X₄ = S; X₅ = Y; X₆ = W and
X₇ = G;
(SEQ ID NO: 113)
WISAYNGNTNYAQKLQG
and
(SEQ ID NO: 153)
DSYWHWWLGA,
X₁ = N; X₂ = A; X₃ = D; X₄ = S; X₅ = Y; X₆ = W and
X₇ = G;
(SEQ ID NO: 25)
WISAYNANTNYAQKLQG
and
(SEQ ID NO: 153)
DSYWHWWLGA,
X₁ = S; X₂ = G; X₃ = D; X₄ = S; X₅ = Y; X₆ = W and
X₇ = G;
(SEQ ID NO: 140)
WISAYSGNTNYAQKLQG
and
(SEQ ID NO: 153)
DSYWHWWLGA,
X₁ = N; X₂ = G; X₃ = D; X₄ = R; X₅ = H; X₆ = W and
X₇ = D;
(SEQ ID NO: 113)
WISAYNGNTNYAQKLQG
and
(SEQ ID NO: 139)
DRHWHWWLDA,
X₁ = N; X₂ = A; X₃ = D; X₄ = R; X₅ = H; X₆ = W and
X₇ = D;
(SEQ ID NO: 25)
WISAYNANTNYAQKLQG
and
(SEQ ID NO: 139)
DRHWHWWLDA,
X₁ = S; X₂ = G; X₃ = D; X₄ = R; X₅ = H; X₆ = W and
X₇ = D;
(SEQ ID NO: 140)
WISAYSGNTNYAQKLQG
and
(SEQ ID NO: 139)
DRHWHWWLDA,
X₁ = N; X₂ = G; X₃ = G; X₄ = Y; X₅ = L; X₆ = D and
X₇ = G;
(SEQ ID NO: 113)
WISAYNGNTNYAQKLQG
and
(SEQ ID NO: 154)
GYLDHWWLGA,
X₁ = N; X₂ = A; X₃ = G; X₄ = Y; X₅ = L; X₆ = D and
X₇ = G;
(SEQ ID NO: 25)
WISAYNANTNYAQKLQG
and
(SEQ ID NO: 154)
GYLDHWWLGA,
or
X₁ = S; X₂ = G; X₃ = G; X₄ = Y; X₅ = L; X₆ = D and
X₇ = G
(SEQ ID NO: 140)
WISAYSGNTNYAQKLQG
and
(SEQ ID NO: 154)
GYLDHWWLGA.
In a preferred embodiment
X₁ = N; X₂ = G; X₃ = D; X₄ = R; X₅ = H; X₆ = W and
X₇ = D;
(SEQ ID NO: 113)
WISAYNGNTNYAQKLQG
and
(SEQ ID NO: 139)
DRHWHWWLDA,
X₁ = N; X₂ = A; X₃ = D; X₄ = R; X₅ = H; X₆ = W and
X₇ = D;
(SEQ ID NO: 25)
WISAYNANTNYAQKLQG
and
(SEQ ID NO: 139)
DRHWHWWLDA,
or
X₁ = S; X₂ = G; X₃ = D; X₄ = R; X₅ = H; X₆ = W and
X₇ = D;
(SEQ ID NO: 140)
WISAYSGNTNYAQKLQG
and
(SEQ ID NO: 139)
DRHWHWWLDA.
Preferably X₁ = N; X₂ = G; X₃ = D; X₄ = R; X₅ = H;
X₆ = W and X₇ = D;
(SEQ ID NO: 113)
WISAYNGNTNYAQKLQG
and
(SEQ ID NO: 139)
DRHWHWWLDA.

The amino acids following the amino acid A in the sequence X₃X₄X₅X₆HWWLX₇A (SEQ ID NO; 26) in the CDR3 sequence of the first variable domain can vary. The amino acid sequence following the sequence X₃X₄X₅X₆HWWLX₇A (SEQ ID NO; 26) can be FDY. The CDR3 of the first variable domain preferably comprises the sequence X₃X₄X₅X₆HWWLX₇AF (SEQ ID NO; 32), preferably X₃X₄X₅X₆HWWLX₇AFD (SEQ ID NO; 33), more preferably X₃X₄X₅X₆HWWLX₇AFDY (SEQ ID NO; 34).

The first variable domain preferably comprises a heavy chain variable region with a CDR1 sequence SYGIS (SEQ ID NO; 24); a CDR2 sequence

WISAYNGNTNYAQKLQG (SEQ ID NO; 113) and a CDR3 sequence X₃X₄X₅X₆HWWLX₇A (SEQ ID NO; 26).

The first variable domain preferably comprises a heavy chain variable region with a CDR1 sequence SYGIS (SEQ ID NO: 24); a CDR2 sequence WISAYNGNTNYAQKLQG (SEQ ID NO: 113) and a CDR3 comprising the sequence DRHWHWWLDA (SEQ ID NO: 139). The amino acids following the sequence LDA in the CDR3 sequence of the first variable domain can vary. The amino acid sequence following the sequence LDA can be FDY. The CDR3 of the first variable domain preferably comprises the sequence DRHWHWWLDAF (SEQ ID NO: 35), preferably DRHWHWWLDAFD (SEQ ID NO: 36), more preferably DRHWHWWLDAFDY (SEQ ID NO: 37).

The first variable domain preferably comprises a heavy chain variable region with the amino acid sequence of MF3353; MF8229; MF8228; MF3370; MF8233; MF8232; MF3393; MF8227 or MF8226 as depicted in FIG. 7 having at most 10, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the indicated sequence. In a preferred embodiment the first variable domain comprises a heavy chain variable region with the amino acid sequence of MF3353; MF8229; MF8228; MF3370; MF8233; MF8232; MF3393; MF8227 or MF8226 as depicted in FIG. 7.

The variable domain that can bind cMET (the second variable domain) preferably comprises a heavy chain variable region that comprises the amino acid sequence of one of the sequences of SEQ ID NO: 1-23 with 0-10 preferably 0-5 amino acid insertions, deletions, substitutions, additions or a combination thereof. The heavy chain variable region of the second variable domain preferably comprises the amino acid sequence of one of the sequences of SEQ ID NO: 1-3; 7; 8; 10; 13; 15; 16; 17; 21; 22 or 23 with 0-10 preferably 0-5 amino acid insertions, deletions, substitutions, additions or a combination thereof. The heavy chain variable region of the second variable domain preferably comprises the amino acid sequence of one of the sequences of SEQ ID NO: 2; 7; 8; 10; 13 or 23 with 0-10 preferably 0-5 amino acid insertions, deletions, substitutions, additions or a combination thereof. The heavy chain variable region of the second variable domain preferably comprises the amino acid sequence of the sequence of SEQ ID NO: 13 or SEQ ID NO: 23 with 0-10, preferably 0-5 amino acid insertions, deletions, substitutions, additions or a combination thereof.

In a preferred embodiment the first variable domain comprises a heavy chain variable region with a CDR1 sequence SYGIS (SEQ ID NO: 24); a CDR2 sequence WISAYNGNTNYAQKLQG (SEQ ID NO: 113) and a CDR3 comprising the sequence DRHWHWWLDA (SEQ ID NO: 35), preferably DRHWHWWLDAFDY (SEQ ID NO: 37) and wherein the second variable domain comprises a heavy chain variable region with a CDR1 sequence SYSMN (SEQ ID NO: 28); a CDR2 sequence WINTYTGDPTYAQGFTG (SEQ ID NO: 29) and a CDR3 sequence ETYYYDRGGYPFDP (SEQ ID NO: 30). The CDR1, CDR2 and CDR3 of a light chain of the first and second variable domain preferably comprises respectively the amino acid sequence CDR1-QSISSY (SEQ ID NO: 38), CDR2-AAS, CDR3-QQSYSTP (SEQ ID NO: 39), i.e. the CDRs of IGKV1-39 (according to IMGT).

In a preferred embodiment the first variable domain comprises a heavy chain variable region with a CDR1 sequence SYGIS (SEQ ID NO: 24); a CDR2 sequence WISAYNGNTNYAQKLQG (SEQ ID NO: 113) and a CDR3 comprising the sequence DRHWHWWLDA (SEQ ID NO: 139) and wherein the second variable domain comprises a heavy chain variable region with a CDR1 sequence TYSMN (SEQ ID NO:152); a CDR2 sequence WINTYTGDPTYAQGFTG (SEQ ID NO: 29) and a CDR3 comprising the sequence ETYFYDRGGYPFDP (SEQ ID NO: 31). The CDR1, CDR2 and CDR3 of a light chain of the first and second variable domain preferably comprises respectively the amino acid sequence CDR1-QSISSY (SEQ ID NO: 38), CDR2-AAS, CDR3-QQSYSTP (SEQ ID NO: 39), i.e. the CDRs of IGKV1-39 (according to IMGT).

A bispecific antibody that comprises a first variable domain that can bind an extracellular part of EGFR and a second variable domain that can bind an extracellular part of cMET wherein the first variable domain comprises a heavy chain variable region with a CDR1 sequence SYGIS (SEQ ID NO: 24); a CDR2 sequence WISAYNANTNYAQKLQG (SEQ ID NO: 25) and a CDR3 comprising the sequence DRHWHWWLDA (SEQ ID NO: 139) and wherein the second variable domain comprises a heavy chain variable region with a CDR1 sequence SYSMN (SEQ ID NO: 28); a CDR2 sequence WINTYTGDPTYAQGFTG (SEQ ID NO: 29) and a CDR3 sequence ETYYYDRGGYPFDP (SEQ ID NO: 30). The CDR1, CDR2 and CDR3 of a light chain of the first and second variable domain preferably comprises respectively the amino acid sequence CDR1-QSISSY (SEQ ID NO: 38), CDR2-AAS, CDR3-QQSYSTP (SEQ ID NO: 39), i.e. the CDRs of IGKV1-39 (according to IMGT).

A bispecific antibody that comprises a first variable domain that can bind an extracellular part of EGFR and a second variable domain that can bind an extracellular part of cMET wherein the first variable domain comprises a heavy chain variable region with a CDR1 sequence SYGIS (SEQ ID NO: 24); a CDR2 sequence WISAYNANTNYAQKLQG (SEQ ID NO: 25) and a CDR3 comprising the sequence DRHWHWWLDA (SEQ ID NO: 139) and wherein the second variable domain comprises a heavy chain variable region with a CDR1 sequence TYSMN (SEQ ID NO: 152); a CDR2 sequence WINTYTGDPTYAQGFTG (SEQ ID NO: 29) and a CDR3 comprising the sequence ETYFYDRGGYPFDP (SEQ ID NO: 31). The CDR1, CDR2 and CDR3 of a light chain of the first and second variable domain preferably comprises respectively the amino acid sequence CDR1-QSISSY (SEQ ID NO: 38), CDR2-AAS, CDR3-QQSYSTP (SEQ ID NO: 39), i.e. the CDRs of IGKV1-39 (according to IMGT).

A bispecific antibody that comprises a first variable domain that can bind an extracellular part of EGFR and a second variable domain that can bind an extracellular part of cMET wherein the first variable domain comprises a heavy chain variable region with a CDR1 sequence SYGIS (SEQ ID NO: 24); a CDR2 sequence WISAYSGNTNYAQKLQG (SEQ ID NO: 140) and a CDR3 comprising the sequence DRHWHWWLDA (SEQ ID NO: 139) and wherein the second variable domain comprises a heavy chain variable region with a CDR1 sequence SYSMN (SEQ ID NO: 28); a CDR2 sequence WINTYTGDPTYAQGFTG (SEQ ID NO: 29) and a CDR3 sequence ETYYYDRGGYPFDP (SEQ ID NO: 30). The CDR1, CDR2 and CDR3 of a light chain of the first and second variable domain preferably comprises respectively the amino acid sequence CDR1-QSISSY (SEQ ID NO: 38), CDR2-AAS, CDR3-QQSYSTP (SEQ ID NO: 39), i.e. the CDRs of IGKV1-39 (according to IMGT).

A bispecific antibody that comprises a first variable domain that can bind an extracellular part of EGFR and a second variable domain that can bind an extracellular part of cMET wherein the first variable domain comprises a heavy chain variable region with a CDR1 sequence SYGIS (SEQ ID NO: 24); a CDR2 sequence WISAYSGNTNYAQKLQG (SEQ ID NO: 140) and a CDR3 comprising the sequence DRHWHWWLDA (SEQ ID NO: 139) and wherein the second variable domain comprises a heavy chain variable region with a CDR1 sequence TYSMN (SEQ ID NO: 152); a CDR2 sequence WINTYTGDPTYAQGFTG (SEQ ID NO: 29) and a CDR3 comprising the sequence ETYFYDRGGYPFDP (SEQ ID NO: 31). The CDR1, CDR2 and CDR3 of a light chain of the first and second variable domain preferably comprises respectively the amino acid sequence CDR1-QSISSY (SEQ ID NO: 38), CDR2-AAS, CDR3-QQSYSTP (SEQ ID NO: 39), i.e. the CDRs of IGKV1-39 (according to IMGT).

The CDR1, CDR2 and CDR3 of a light chain of the first and second variable domain as described herein preferably comprises respectively the amino acid sequence CDR1-QSISSY (SEQ ID NO: 38), CDR2-AAS, CDR3-QQSYSTP (SEQ ID NO: 39), i.e. the CDRs of IGKV1-39 (according to IMGT). In some embodiments of a bispecific antibody as described herein the first and second variable domain comprise a common light chain, preferably a light chain of FIG. 9B.

In another preferred embodiment an EGFR/cMET bispecific antibody comprises a first variable domain that can bind an extracellular part of human EGFR that comprises the CDR1, CDR2 and CDR3 of the heavy chain variable region of MF3755 depicted in FIG. 1 and a second variable domain that can bind an extracellular part of human cMET that comprises the CDR1, CDR2 and CDR3 of the heavy chain variable region of MF4297 depicted in FIG. 1. The light chain variable region in said first and second variable domain is preferably a common light chain variable region as described herein. The CDR1, CDR2 and CDR3 of a light chain of the first and second variable domain preferably comprises respectively the amino acid sequence CDR1-QSISSY (SEQ ID NO: 38), CDR2-AAS, CDR3-QQSYSTP (SEQ ID NO: 39), i.e. the CDRs of IGKV1-39 (according to IMGT). In a preferred embodiment the antibody comprises a heavy chain variable region with the amino acid sequence of MF3755 as depicted in FIG. 1 having at most 10, preferably 0, 1, 2, 3, 4, 5, 6, 7, 8, 9 or 10 and preferably having 0, 1, 2, 3, 4 or 5 amino acid insertions, deletions, substitutions or a combination thereof with respect to the indicated sequence. In a preferred embodiment the first variable domain comprises a heavy chain variable region with the amino acid sequence of MF3755 as depicted in FIG. 1. The variable domain that can bind cMET (the second variable domain) preferably comprises a heavy chain variable region that comprises the amino acid sequence of MF4297 as depicted in FIG. 1 with 0-10 preferably 0-5 amino acid insertions, deletions, substitutions, additions or a combination thereof. The heavy chain variable region of the second variable domain preferably comprises the amino acid sequence of MF4297 as depicted in FIG. 1.

The term ‘bispecific’ (bs) in the context of the present invention means that an antibody is capable of binding two different targets or two epitopes on the same target, for example, where one variable domain of the antibody (as defined above) binds to an epitope on EGFR and a second variable domain binds to an epitope on cMET. Depending on the expression level, (sub-)cellular localization and stoichiometry of the two antigens recognized by a bispecific antibody, both Fab arms of the antibody may or may not simultaneously bind their epitope. One arm of the bispecific antibody typically contains the variable domain of one antibody and the other arm contains the variable domain of another antibody (i.e. one arm of the bispecific antibody is formed by one heavy chain paired with one light chain whereas the other arm is formed by a different heavy chain paired with a light chain). Thus, the stoichiometry of a preferred bispecific antibody of an invention disclosed herein is 1:1, EGFR:cMET binding.

The heavy chain variable regions of the bispecific antibody of an invention as disclosed herein are typically different from each other, whereas the light chain variable regions are preferably the same. A bispecific antibody wherein the different heavy chain variable regions are associated with the same light chain variable region is also referred to as a bispecific antibody with a common light chain variable region (cLcv). It is preferred that the light chain constant region is also the same. Such bispecific antibodies are referred to as having a common light chain (cLc). Further provided is therefore a bispecific antibody according to an invention as disclosed herein, wherein both arms comprise a common light chain.

The term ‘common light chain’ according to an invention disclosed herein refers to two or more light chains in a bispecific antibody which may be identical or have some amino acid sequence differences while the binding specificity of the full length antibody is not affected. It is for instance possible within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like. The terms ‘common light chain’, ‘common LC’, ‘cLC’, ‘single light chain’ with or without the addition of the term ‘rearranged’ are all used herein interchangeably. The terms ‘common light chain variable region’, ‘common VL’, ‘common LCv’, ‘cLCv’, ‘single VL’ with or without the addition of the term ‘rearranged’ are all used herein interchangeably. It is a preferred aspect of the present invention that a bispecific antibody has a common light chain (variable region) that can combine with at least two, and preferably a plurality of heavy chains (variable regions) of different binding specificity to form antibodies with functional antigen binding domains (e.g., WO2009/157771). The common light chain (variable region) is preferably a human light chain (variable region). A common light chain (variable region) preferably has a germline sequence. A preferred germline sequence is a light chain variable region that has good thermodynamic stability, yield and solubility. A preferred germline light chain is O12. A common light chain preferably comprises the light chain encoded by a germline human Vk gene segment, and is preferably the rearranged germline human kappa light chain IgV_{K}1-39*01/IGJ_{K}1*01 (FIG. 9A). The common light chain variable region is preferably the variable region of the rearranged germline human kappa light chain IgV_{K}1-39*01/IGJ_{K}1*01. A common light chain preferably comprises a light chain variable region as depicted in FIG. 9B, or 9D with 0-5 amino acid insertions, deletions, substitutions, additions or a combination thereof. The common light preferably further comprises a light chain constant region, preferably a kappa light chain constant region. A nucleic acid that encodes the common light chain can be codon optimized for the cell system used to express the common light chain protein. The encoding nucleic acid can deviate from a germ-line nucleic acid sequence.

In a preferred embodiment the light chain comprises a light chain region comprising the amino acid sequence of an O12/IgV_{K}1-39*01 gene segment as depicted in FIG. 9A with 0-10, preferably 0-5 amino acid insertions, deletions, substitutions, additions or a combination thereof. The phrase “O12 light chain” will be used throughout the specification as short for “a light chain comprising a light chain variable region comprising the amino acid sequence of an O12/IgV_{K}1-39*01 gene segment as depicted in FIG. 9A with 0-10, preferably 0-5 amino acid insertions, deletions, substitutions, additions or a combination thereof. IgV_{K}1-39 is short for Immunoglobulin Variable Kappa 1-39 Gene. The gene is also known as Immunoglobulin Kappa Variable 1-39; IGKV139; IGKV1-39; O12a or O12. External Ids for the gene are HGNC: 5740; Entrez Gene: 28930; Ensembl: ENSG00000242371. A preferred amino acid sequence for IgV_{K}1-39 is given in FIG. 9E. This lists the sequence of the V-region. The V-region can be combined with one of five J-regions. FIG. 9B and 9D describe two preferred sequences for IgV_{K}1-39 in combination with a J-region. The joined sequences are indicated as IGKV1-39/jk1 and IGKV1-39/jk5; alternative names are IgV_{K}1-39*01/IGJ_{K}1*01 or IgV_{K}1-39*01/IGJ_{K}5*01 (nomenclature according to the IMGT database worldwide web at imgt.org).

It is preferred that the O12/IgV_{K}1-39*01 comprising light chain variable region is a germline sequence. It is further preferred that the IGJ_{K}1*01 or/IGJ_{K}5*01 comprising light chain variable region is a germline sequence. In a preferred embodiment, the IGKV1-39/jk1 or IGKV1-39/jk5 light chain variable regions are germline sequences.

In a preferred embodiment the light chain variable region comprises a germline O12/IgV_{K}1-39*01. In a preferred embodiment the light chain variable region comprises the kappa light chain IgV_{K}1-39*01/IGJ_{K}1*01 or IgV_{K}1-39*01/IGJ_{K}5*01. In a preferred embodiment a IgV_{K}1(1-39*01/IGJ_{K}1*01. The light chain variable region preferably comprises a germline kappa light chain IgV_{K}1(1-39*01/IGJ_{K}1*01 or germline kappa light chain IgV_{K}1-39*01/IGJ_{K}5*01, preferably a germline IgV_{K}1-39*01/IGJ_{K}1*01.

Mature B-cells that produce an antibody with an O12 light chain often produce a light chain that has undergone one or more mutations with respect to the germline sequence, i.e. the normal sequence in non-lymphoid cells of the organism. The process that is responsible for these mutations is often referred to as somatic (hyper)mutation.

The resulting light chain is referred to as an affinity matured light chain. Such light chains, when derived from an O12 germline sequence are O12-derived light chains. In this specification, the phrase “common light chain” will include “common light chain derived light chains and the phrase “O12 light chains” will include O12-derived light chains. The mutations that are introduced by somatic hypermutation can also be introduced artificially in the lab. In the lab also other mutations can be introduced without affecting the properties of the light chain in kind, not necessarily in amount. A light chain is at least an O12 light chain if it comprises a sequence as depicted in FIG. 9A, FIG. 9B; FIG. 9D or FIG. 9E with 0-10, preferably 0-5 amino acid insertions, deletions, substitutions, additions or a combination thereof. In a preferred embodiment the O12 light chain is a light chain comprising a sequence as depicted in FIG. 9A; 9B; 9D or 9E with 0-9, 0-8, 0-7, 0-6, 0-5, 0-4 amino acid insertions, deletions, substitutions, additions or a combination thereof. In a preferred embodiment the O12 light chain is a light chain comprising a sequence as depicted in FIG. 9A, FIG. 9B; FIG. 9D or FIG. 9E with 0-5, preferably 0-4, more preferably 0-3 amino acid insertions, deletions, substitutions, additions or a combination thereof. In a preferred embodiment the O12 light chain is a light chain comprising a sequence as depicted in FIG. 9A, FIG. 9B; FIG. 9D or FIG. 9E with 0-2, more preferably 0-1, most preferably 0 amino acid insertions, deletions, substitutions, additions or a combination thereof. In a preferred embodiment the O12 light chain is a light chain comprising a sequence as depicted in FIG. 9A or FIG. 9B with the mentioned amino acid insertions, deletions, substitutions, additions or a combination thereof. In a preferred embodiment the light chain comprises the sequence of FIG. 9A. In a preferred embodiment the light chain variable region comprises the sequence of FIG. 9B. The mentioned 1, 2, 3, 4 or 5 amino acid substitutions are preferably conservative amino acid substitutions, the insertions, deletions, substitutions or combination thereof are preferably not in the CDR3 region of the VL chain, preferably not in the CDR1, CDR2 or CDR3 region or FR4 region of the VL chain.

The common light chain can have a lambda light chain and this is therefore also provided in the context of an invention as disclosed herein, however a kappa light chain is preferred. The constant part of a common light chain of an invention as disclosed herein can be a constant region of a kappa or a lambda light chain. It is preferably a constant region of a kappa light chain, preferably wherein said common light chain is a germline light chain, preferably a rearranged germline human kappa light chain comprising the IgVK1-39 gene segment, most preferably the rearranged germline human kappa light chain IgVK1-39*01/IGJK1*01 (FIG. 9). The terms rearranged germline human kappa light chain IgV_{K}1-39*01/IGJ_{K}1*01, IGKV1-39/IGKJ1, huV_{K}1-39 light chain or in short huV_{K}1-39, or simply 1-39 are used interchangeably throughout the application.

A cell that produces a common light chain can produce for instance rearranged germline human kappa light chain IgV_{K}1-39*01/IGJ_{K}1*01 and a light chain comprising the variable region of the mentioned light chain fused to a lambda constant region.

In a preferred embodiment the light chain variable region comprises the amino acid sequence DIQMT QSPSS LSASV GDRVT ITCRA SQSIS SYLNW YQQKP GKAPK LLIYA ASSLQ SGVPS RFSGS GSGTD FTLTI SSLQP EDFAT YYCQQ SYSTP PTFGQ GTKVE IK (SEQ ID NO: 40) or DIQMT QSPSS LSASV GDRVT ITCRA SQSIS SYLNW YQQKP GKAPK LLIYA ASSLQ SGVPS RFSGS GSGTD FTLTI SSLQP EDFAT YYCQQ SYSTP PITFG QGTRL EIK (SEQ ID NO: 41) with 0-10, preferably 0-5 amino acid insertions, deletions, substitutions, additions or a combination thereof. In a preferred embodiment the light chain variable region comprises 0-9, 0-8, 0-7, 0-6, 0-5, 0-4, preferably 0-3, preferably 0-2, preferably 0-1 and preferably 0 amino acid insertions, deletions, substitutions, additions with respect to the indicated amino acid sequence, or a combination thereof. A combination of an insertion, deletion, addition or substitution is a combination as claimed if aligned sequences do not differ at more than 5 positions. In a preferred embodiment the light chain variable region comprises the amino acid sequence DIQMT QSPSS LSASV GDRVT ITCRA SQSIS SYLNW YQQKP GKAPK LLIYA ASSLQ SGVPS RFSGS GSGTD FTLTI SSLQP EDFAT YYCQQ SYSTP PTFGQ GTKVE IK (SEQ ID NO: 40) or DIQMT QSPSS LSASV GDRVT ITCRA SQSIS SYLNW YQQKP GKAPK LLIYA ASSLQ SGVPS RFSGS GSGTD FTLTI SSLQP EDFAT YYCQQ SYSTP PITFG QGTRL EIK (SEQ ID NO: 41). In a preferred embodiment the light chain variable region comprises the amino acid sequence DIQMT QSPSS LSASV GDRVT ITCRA SQSIS SYLNW YQQKP GKAPK LLIYA ASSLQ SGVPS RFSGS GSGTD FTLTI

SSLQP EDFAT YYCQQ SYSTP PTFGQ GTKVE IK (SEQ ID NO: 40). In another preferred embodiment the light chain variable region comprises the amino acid sequence DIQMT QSPSS LSASV GDRVT ITCRA SQSIS SYLNW YQQKP GKAPK LLIYA ASSLQ SGVPS RFSGS GSGTD FTLTI SSLQP EDFAT YYCQQ SYSTP PITFG QGTRL EIK (SEQ ID NO: 41).

The amino acid insertions, deletions, substitutions, additions or combination thereof are preferably not in the CDR3 region of the light chain variable region, preferably not in the CDR1 or CDR2 region of the light chain variable region. In a preferred embodiment the light chain variable region does not comprise a deletion, addition or insertion with respect to the sequence indicated. In this embodiment the heavy chain variable region can have 0-5 amino acid substitutions with respect to the indicated amino acid sequence. An amino acid substitution is preferably a conservative amino acid substitution. The CDR1, CDR2 and CDR3 of a light chain of an antibody of the invention preferably comprises respectively the amino acid sequence CDR1-QSISSY (SEQ ID NO: 38), CDR2-AAS, CDR3-QQSYSTP (SEQ ID NO: 39), i.e. the CDRs of IGKV1-39 (according to IMGT).

Bispecific antibodies as described herein preferably have one heavy chain variable region/light chain variable region (VH/VL) combination that binds an extracellular part of EGFR and a second VH/VL combination that binds an extracellular of cMET. In a preferred embodiment the VL in said first VH/VL combination is similar to the VL in said second VH/VL combination. In a more preferred embodiment, the VLs in the first and second VH/VL combinations are identical. In a preferred embodiment, the bispecific antibody is a full length antibody which has one heavy/light (H/L) chain combination that binds an extracellular part of EGFR and one H/L chain combination that binds an extracellular part of cMET. In a preferred embodiment the light chain in said first H/L chain combination is similar to the light chain in said second H/L chain combination. In a more preferred embodiment, the light chains in the first and second H/L chain combinations are identical.

Several methods have been published to produce a host cell whose expression favors the production of the bispecific antibody or vice versa, the monospecific antibodies. In the present invention it is preferred that the cellular expression of the antibody molecules is favored toward the production of the bispecific antibody over the production of the respective monospecific antibodies. Such is typically achieved by modifying the constant region of the heavy chains such that they favor heterodimerization (i.e. dimerization with the heavy chain of the other heavy/light chain combination) over homodimerization. In a preferred embodiment the bispecific antibody of an invention as disclosed herein comprises two different immunoglobulin heavy chains with compatible heterodimerization domains. Various compatible heterodimerization domains have been described in the art. The compatible heterodimerization domains are preferably compatible immunoglobulin heavy chain CH3 heterodimerization domains. When wildtype CH3 domains are used, co-expression of two different heavy chains (A and B) and a common light chain will result in three different antibody species, AA, AB and BB. AA and BB are designations for the two mono-specific, bivalent antibodies, and AB is a designation for the bispecific antibody. To increase the percentage of the desired bispecific product (AB) CH3 engineering can be employed, or in other words, one can use heavy chains with compatible hetero-dimerization domains, as defined hereunder. The art describes various ways in which such hetero-dimerization of heavy chains can be achieved. One way is to generate ‘knob into hole’ bispecific antibodies.

The term ‘compatible hetero-dimerization domains’ as used herein refers to protein domains that are engineered such that engineered domain A′ will preferentially form heterodimers with engineered domain B′ and vice versa, homo-dimerization between A′-A′ and B′-B′ is diminished.

In U.S. Ser. No. 13/866,747 (now issued as U.S. Pat. No. 9,248,181), U.S. Ser. No. 14/081,848 (now issued as U.S. Pat. No. 9,358,286) and PCT/NL2013/050294 (published as WO2013/157954; incorporated herein by reference) methods and means are disclosed for producing bispecific antibodies using compatible heterodimerization domains. These means and methods can also be favorably employed in the present invention. Specifically, a bispecific antibody of an invention as disclosed herein preferably comprises mutations to produce substantial expression of bispecific full length IgG molecules in host cells. Preferred mutations are the amino acid substitutions L351K and T366K in the first CH3 domain (the ‘KK-variant’ heavy chain) and the amino acid substitutions L351D and L368E in the second domain (the ‘DE-variant’ heavy chain), or vice versa. U.S. Pat. Nos. 9,248,181 and 9,358,286 patents as well as the WO2013/157954 PCT application (which are incorporated by reference herein) demonstrate that the DE-variant and KK-variant preferentially pair to form heterodimers (so-called ‘DEKK’ bispecific molecules). Homodimerization of DE-variant heavy chains (DEDE homodimers) are disfavored due to repulsion between the charged residues in the CH3-CH3 interface between identical heavy chains.

Bispecific antibodies can be generated by (transient) transfection of plasmids encoding a light chain and two different heavy chains that are CH3 engineered to ensure efficient hetero-dimerization and formation of the bispecific antibodies. The production of these chains in a single cell leads to the favored formation of bispecific antibodies over the formation of monospecific antibodies. Preferred mutations to produce essentially only bispecific full length IgG1 molecules are amino acid substitutions at positions 351 and 366, e.g. L351K and T366K (numbering according to EU numbering) in the first CH3 domain (the ‘KK-variant’ heavy chain) and amino acid substitutions at positions 351 and 368, e.g. L351D and L368E in the second CH3 domain (the ‘DE-variant’ heavy chain), or vice versa (see for instance FIGS. 10E and 10F).

In one embodiment the heavy chain/light chain combination that comprises the variable domain that binds EGFR, comprises a DE variant of the heavy chain. In this embodiment the heavy chain/light chain combination that comprises the variable domain that can bind to cMET comprises a KK variant of the heavy chain. The KK variant of the heavy chain that binds cMET do not produce homodimers thereby rendering the observed effect of HGF induced cMET activation inhibition by the bispecific antibody very precise. It avoids activation of cMET sometimes observed with bivalent cMET antibodies (agonism).

The Fc region mediates effector functions of an antibody, such as complement-dependent cytotoxicity (CDC), antibody-dependent cellular cytotoxicity (ADCC) and antibody-dependent cell phagocytosis (ADCP). Depending on the therapeutic antibody or Fc fusion protein application, it may be desired to either reduce or increase the effector function. Reduced effector function can be desired when an immune response is to be activated, enhanced or stimulated as in some of the embodiments of an invention as disclosed herein. Antibodies with reduced effector functions can be used to target cell-surface molecules of immune cells, among others.

Antibodies with reduced effector functions are preferably IgG antibodies comprising a modified CH2/lower hinge region, for instance to reduce Fc-receptor interaction or to reduce C1q binding. In some embodiments the antibody of the invention is an IgG antibody with a mutant CH2 and/or lower hinge domain such that interaction of the bispecific IgG antibody to a Fc-gamma receptor is reduced. An antibody comprising a mutant CH2 region is preferably an IgG1 antibody. Such a mutant IgG1 CH2 and/or lower hinge domain preferably comprise an amino substitution at position 235 and/or 236 (EU-numbering), preferably an L235G and/or G236R substitution (FIG. 10D).

An antibody of an invention as disclosed herein preferably has effector function. A bispecific antibody of an invention as disclosed herein preferably comprises antibody-dependent cell-mediated cytotoxicity (ADCC). The antibody can be engineered to enhance the ADCC activity (for review, see Cancer Sci. 2009 September; 100(9)1566-72. Engineered therapeutic antibodies with improved effector functions. Kubota T, Niwa R, Satoh M, Akinaga S, Shitara K, Hanai N). Several in vitro methods exist for determining the efficacy of antibodies or effector cells in eliciting ADCC. Among these are chromium-51 [Cr51] release assays, europium [Eu] release assays, and sulfur-35 [S35] release assays. Usually, a labeled target cell line expressing a certain surface-exposed antigen is incubated with antibody specific for that antigen. After washing, effector cells expressing Fc receptor CD16 are co-incubated with the antibody-labeled target cells. Target cell lysis is subsequently measured by release of intracellular label by a scintillation counter or spectrophotometry. In one aspect a bispecific antibody of an invention as disclosed herein exhibits ADCC activity. In such aspect the bispecific antibody can have improved ADCC activity. In another aspect a bispecific antibody of an invention as disclosed herein does not exhibit ADCC activity. In such aspect the antibody can have reduced ADCC by means of one or more CH2 mutations as described elsewhere herein and by techniques known to in the art. One technique for enhancing ADCC of an antibody is afucosylation. (See for instance Junttila, T. T., K. Parsons, et al. (2010). “Superior In vivo Efficacy of Afucosylated Trastuzumab in the Treatment of HER2-Amplified Breast Cancer.” Cancer Research 70(11): 4481-4489). Further provided is therefore a bispecific antibody according to an invention as disclosed herein, which is afucosylated. Alternatively, or additionally, multiple other strategies can be used to achieve ADCC enhancement, for instance including glycoengineering (Kyowa Hakko/Biowa, GlycArt (Roche) and Eureka Therapeutics) and mutagenesis, all of which seek to improve Fc binding to low-affinity activating FcγRIIIa, and/or to reduce binding to the low affinity inhibitory FcγRIIb. A bispecific antibody of an invention as disclosed herein is preferably afucosylated in order to enhance ADCC activity. A bispecific antibody of an invention as disclosed herein preferably comprises a reduced amount of fucosylation of the N-linked carbohydrate structure in the Fc region, when compared to the same antibody produced in a normal CHO cell.

A variant of an antibody or bispecific antibody as described herein comprises a functional part, derivative and/or analogue of the antibody or bispecific antibody. The variant maintains the binding specificity of the (bispecific) antibody. The functional part, derivative and/or analogue maintains the binding specificity of the (bispecific) antibody. Binding specificity is defined by capacity to bind an extracellular part of a first membrane protein and a second membrane protein as described herein.

A bispecific antibody of an invention disclosed herein is preferably used in humans. A preferred antibody of the invention is a human or humanized antibody. The constant region of a bispecific antibody of an invention disclosed herein is preferably a human constant region. The constant region may contain one or more, preferably not more than 10, preferably not more than 5 amino-acid differences with the constant region of a naturally occurring human antibody. It is preferred that the constant part is entirely derived from a naturally occurring human antibody. Various antibodies produced herein are derived from a human antibody variable domain library. As such these variable domains are human. The unique CDR regions may be derived from humans, be synthetic or derived from another organism. The variable region is considered a humanized variable region when it has an amino acid sequence that is identical to an amino acid sequence of the variable region of a naturally occurring human antibody, but for the CDR regions. In such embodiments, the VH of a variable domain of an antibody that binds EGFR or cMET of an invention as disclosed herein may contain one or more, preferably not more than 10, preferably not more than 5 amino-acid differences with the variable region of a naturally occurring human antibody, not counting possible differences in the amino acid sequence of the CDR regions. The light chain variable region of an EGFR binding domain and/or a cMET binding domain in an antibody of invention as disclosed herein may contain one or more, preferably not more than 10, preferably not more than 5 amino-acid differences with the variable region of a naturally occurring human antibody, not counting possible differences in the amino acid sequence of the CDR regions. The light chain in an antibody of an invention as disclosed herein may contain one or more, preferably not more than 10, preferably not more than 5 amino-acid differences with the variable region of a naturally occurring human antibody, not counting possible differences in the amino acid sequence of the CDR regions. Such mutations also occur in nature in the context of somatic hypermutation.

Antibodies may be derived from various animal species, at least with regard to the heavy chain variable region. It is common practice to humanize such e.g. murine heavy chain variable regions. There are various ways in which this can be achieved among which there are CDR-grafting into a human heavy chain variable region with a 3D-structure that matches the 3-D structure of the murine heavy chain variable region; deimmunization of the murine heavy chain variable region, preferably done by removing known or suspected T- or B-cell epitopes from the murine heavy chain variable region. The removal is typically by substituting one or more of the amino acids in the epitope for another (typically conservative) amino acid, such that the sequence of the epitope is modified such that it is no longer a T- or B-cell epitope.

Deimmunized murine heavy chain variable regions are less immunogenic in humans than the original murine heavy chain variable region. Preferably a variable region or domain of an invention as disclosed herein is further humanized, such as for instance veneered. By using veneering techniques, exterior residues which are readily encountered by the immune system are selectively replaced with human residues to provide a hybrid molecule that comprises either a weakly immunogenic or substantially non-immunogenic veneered surface. An animal as used in an invention as disclosed herein is preferably a mammal, more preferably a primate, most preferably a human.

A bispecific antibody according to an invention as disclosed herein preferably comprises a constant region of a human antibody. According to differences in their heavy chain constant domains, antibodies are grouped into five classes, or isotypes: IgG, IgA, IgM, IgD, and IgE. These classes or isotypes comprise at least one of said heavy chains that is named with a corresponding Greek letter. A preferred embodiment comprises an antibody wherein said constant region is selected from the group of IgG, IgA, IgM, IgD, and IgE constant regions, more preferably said constant region comprises an IgG constant region, i.e. selected from the group consisting of IgG1, IgG2, IgG3 and IgG4. Preferably, said constant region is an IgG1 or IgG4 constant region, more preferably a mutated IgG1 constant region. Some variation in the constant region of

IgG1 occurs in nature and/or is allowed without changing the immunological properties of the resulting antibody. Variation can also be introduced artificially to install certain preferred features on the antibody or parts thereof. Such features are for instance described herein in the context of CH2 and CH3. Typically between about 1-10 amino acid insertions, deletions, substitutions or a combination thereof are allowed in the constant region.

A VH chain of FIG. 1, 7 or 8 preferably has at most 15, preferably 0, 1, 2, 3, 4, 5, 6, 7, 8, 9 or 10 amino acid insertions, deletions, substitutions or a combination thereof with respect to the VH chain depicted in FIG. 1, 7 or 8, preferably has 0, 1, 2, 3, 4 or 5 10 amino acid insertions, deletions, substitutions or a combination thereof with respect to the VH chain depicted in FIG. 1, 7 or 8, preferably 0, 1, 2, 3 or 4 insertions, deletions, substitutions or a combination thereof, preferably 0, 1, 2 or 3 insertions, deletions, substitutions or a combination thereof, more preferably 0; 1 or 2 insertions, deletions, substitutions or a combination thereof, and preferably 0 or 1 insertion, deletion, substitution or a combination thereof with respect to the VH chain depicted in FIG. 1, 7 or 8. The one or more amino acid insertions, deletions, substitutions or a combination thereof are preferably not in the CDR1, CDR2 and/or CDR3 region of the VH chain. They are also preferably not present in the Fr4 region. An amino acid substitution is preferably a conservative amino acid substitution.

Rational methods have evolved toward minimizing the content of non-human residues in the human context. Various methods are available to successfully graft the antigen-binding property of an antibody onto another antibody. The binding properties of antibodies may rest predominantly in the exact sequence of the CDR3 region, often supported by the sequence of the CDR1 and CDR2 regions in the variable domain combined with the appropriate structure of the variable domain as a whole. The amino acid sequence of a CDR region as depicted herein determined with the Kabat definition. Various methods are presently available to graft CDR regions onto a suitable variable domain of another antibody. Some of these methods are reviewed in J. C. Almagrol and J. Fransson (2008) Frontiers in Bioscience 13, 1619-1633, which is included by reference herein. An invention as disclosed herein therefore further provides a human or humanized bispecific antibody comprising a first antigen-binding site that binds EGFR and a second antigen-binding site that binds cMET, wherein the variable domain comprising the EGFR binding site comprises a VH CDR3 sequence as depicted for MF3370 in FIG. 1, and wherein the variable domain comprising the cMET binding site comprises a VH CDR3 region as depicted for MF4356 in FIG. 1. The VH variable region comprising the EGFR binding site preferably comprises the sequence of the CDR1 region, CDR2 region and the CDR3 region of a VH chain as depicted for MF3370 in FIG. 1. The VH variable region comprising the cMET binding site preferably comprises the sequence of the CDR1 region, CDR2 region and the CDR3 region of a VH chain as depicted for MF4356 in FIG. 1. CDR grafting may also be used to produce a VH chain with the CDR regions of a VH of FIG. 1, but having a different framework. The different framework may be of another human VH, or of a different mammal. An invention as disclosed herein therefore further provides a human or humanized bispecific antibody comprising a first antigen-binding site that binds EGFR and a second antigen-binding site that binds cMET, wherein the variable domain comprising the EGFR binding site comprises a VH CDR3 sequence as depicted for MF8233 in FIG. 7, and wherein the variable domain comprising the cMET binding site comprises a VH CDR3 region as depicted for MF8230 in FIG. 8. The VH variable region comprising the EGFR binding site preferably comprises the sequence of the CDR1 region, CDR2 region and the CDR3 region of a VH chain as depicted for MF8233 in FIG. 7. The VH variable region comprising the cMET binding site preferably comprises the sequence of the CDR1 region, CDR2 region and the CDR3 region of a VH chain as depicted for MF8230 in FIG. 8. CDR grafting may also be used to produce a VH chain with the CDR regions of a VH of FIG. 7 or FIG. 8, but having a different framework. The different framework may be of another human VH, or of a different mammal

An invention as disclosed herein therefore further provides a human or humanized bispecific antibody comprising a first antigen-binding site that binds EGFR and a second antigen-binding site that binds cMET, wherein the variable domain comprising the EGFR binding site comprises a VH CDR3 sequence as depicted for MF3370 in FIG. 1, and wherein the variable domain comprising the cMET binding site comprises a VH CDR3 region as depicted for MF8230 in FIG. 8. The VH variable region comprising the EGFR binding site preferably comprises the sequence of the CDR1 region, CDR2 region and the CDR3 region of a VH chain as depicted for MF3370 in FIG. 1. The VH variable region comprising the cMET binding site preferably comprises the sequence of the CDR1 region, CDR2 region and the CDR3 region of a VH chain as depicted for MF8230 in FIG. 8. CDR grafting may also be used to produce a VH chain with the CDR regions of a VH of FIG. 7 or FIG. 8, but having a different framework. The different framework may be of another human VH, or of a different mammal

An invention as disclosed herein therefore further provides a human or humanized bispecific antibody comprising a first antigen-binding site that binds EGFR and a second antigen-binding site that binds cMET, wherein the variable domain comprising the EGFR binding site comprises a VH CDR3 sequence as depicted for MF8233 in FIG. 7, and wherein the variable domain comprising the cMET binding site comprises a VH CDR3 region as depicted for MF4356 in FIG. 8. The VH variable region comprising the EGFR binding site preferably comprises the sequence of the CDR1 region, CDR2 region and the CDR3 region of a VH chain as depicted for MF8233 in FIG. 7. The VH variable region comprising the cMET binding site preferably comprises the sequence of the CDR1 region, CDR2 region and the CDR3 region of a VH chain as depicted for MF4356 in FIG. 8. CDR grafting may also be used to produce a VH chain with the CDR regions of a VH of FIG. 7 or FIG. 8, but having a different framework. The different framework may be of another human VH, or of a different mammal

An invention as disclosed herein therefore further provides a human or humanized bispecific antibody comprising a first antigen-binding site that binds EGFR and a second antigen-binding site that binds cMET, wherein the variable domain comprising the EGFR binding site comprises a VH CDR3 sequence as depicted for

MF8232 in FIG. 7, and wherein the variable domain comprising the cMET binding site comprises a VH CDR3 region as depicted for MF8230 in FIG. 8. The VH variable region comprising the EGFR binding site preferably comprises the sequence of the CDR1 region, CDR2 region and the CDR3 region of a VH chain as depicted for MF8232 in FIG. 7. The VH variable region comprising the cMET binding site preferably comprises the sequence of the CDR1 region, CDR2 region and the CDR3 region of a VH chain as depicted for MF8230 in FIG. 8. CDR grafting may also be used to produce a VH chain with the CDR regions of a VH of FIG. 7 or FIG. 8, but having a different framework. The different framework may be of another human VH, or of a different mammal

An invention as disclosed herein therefore further provides a human or humanized bispecific antibody comprising a first antigen-binding site that binds EGFR and a second antigen-binding site that binds cMET, wherein the variable domain comprising the EGFR binding site comprises a VH CDR3 sequence as depicted for MF8232 in FIG. 7, and wherein the variable domain comprising the cMET binding site comprises a VH CDR3 region as depicted for MF4356 in FIG. 8. The VH variable region comprising the EGFR binding site preferably comprises the sequence of the CDR1 region, CDR2 region and the CDR3 region of a VH chain as depicted for MF8232 in FIG. 7. The VH variable region comprising the cMET binding site preferably comprises the sequence of the CDR1 region, CDR2 region and the CDR3 region of a VH chain as depicted for MF4356 in FIG. 8. CDR grafting may also be used to produce a VH chain with the CDR regions of a VH of FIG. 7 or FIG. 8, but having a different framework. The different framework may be of another human VH, or of a different mammal

The invention further provides a human or humanized bispecific antibody comprising a first variable domain that binds EGFR and a second variable domain that binds cMET wherein the first variable domain comprises a heavy chain variable region with the amino acid sequence of MF3370 as depicted in FIG. 7 having at most 10, preferably 0, 1, 2, 3, 4, 5, 6, 7, 8, 9 or 10 and preferably having 0, 1, 2, 3, 4 or 5 amino acid insertions, deletions, substitutions or a combination thereof and wherein the second variable domain comprises a heavy chain variable region that comprises the amino acid sequence of MF4356 depicted in FIG. 8 (SEQ ID NO: 23) with 0-10 preferably 0-5 amino acid insertions, deletions, substitutions, additions or a combination thereof. The invention further provides a human or humanized bispecific antibody comprising a first variable domain that binds EGFR and a second variable domain that binds cMET wherein the first variable domain comprises a heavy chain variable region with the amino acid sequence of MF8233 as depicted in FIG. 7 having at most 10, preferably 0, 25 1, 2, 3, 4, 5, 6, 7, 8, 9 or 10 and preferably having 0, 1, 2, 3, 4 or 5 amino acid insertions, deletions, substitutions or a combination thereof and wherein the second variable domain comprises a heavy chain variable region that comprises the amino acid sequence of MF8230 depicted in FIG. 8 (SEQ ID NO: 13) with 0-10 preferably 0-5 amino acid insertions, deletions, substitutions, additions or a combination thereof.

The invention further provides a human or humanized bispecific antibody comprising a first variable domain that binds EGFR and a second variable domain that binds cMET wherein the first variable domain comprises a heavy chain variable region with the amino acid sequence of MF3370 as depicted in FIG. 7 having at most 10, preferably 0, 1, 2, 3, 4, 5, 6, 7, 8, 9 or 10 and preferably having 0, 1, 2, 3, 4 or 5 amino acid insertions, deletions, substitutions or a combination thereof and wherein the second variable domain comprises a heavy chain variable region that comprises the amino acid sequence of MF8230 depicted in FIG. 8 (SEQ ID NO: 13) with 0-10 preferably 0-5 amino acid insertions, deletions, substitutions, additions or a combination thereof.

The invention further provides a human or humanized bispecific antibody comprising a first variable domain that binds EGFR and a second variable domain that binds cMET wherein the first variable domain comprises a heavy chain variable region with the amino acid sequence of MF8233 as depicted in FIG. 7 having at most 10, preferably 0, 1, 2, 3, 4, 5, 6, 7, 8, 9 or 10 and preferably having 0, 1, 2, 3, 4 or 5 amino acid insertions, deletions, substitutions or a combination thereof and wherein the second variable domain comprises a heavy chain variable region that comprises the amino acid sequence of MF4356 depicted in FIG. 8 (SEQ ID NO: 23) with 0-10 preferably 0-5 amino acid insertions, deletions, substitutions, additions or a combination thereof.

The invention further provides a human or humanized bispecific antibody comprising a first variable domain that binds EGFR and a second variable domain that binds cMET wherein the first variable domain comprises a heavy chain variable region with the amino acid sequence of MF8232 as depicted in FIG. 7 having at most 10, preferably 0, 1, 2, 3, 4, 5, 6, 7, 8, 9 or 10 and preferably having 0, 1, 2, 3, 4 or 5 amino acid insertions, deletions, substitutions or a combination thereof and wherein the second variable domain comprises a heavy chain variable region that comprises the amino acid sequence of MF4356 depicted in FIG. 8 (SEQ ID NO: 23) with 0-10 preferably 0-5 amino acid insertions, deletions, substitutions, additions or a combination thereof.

The invention further provides a human or humanized bispecific antibody comprising a first variable domain that binds EGFR and a second variable domain that binds cMET wherein the first variable domain comprises a heavy chain variable region with the amino acid sequence of MF8232 as depicted in FIG. 7 having at most 10, preferably 0, 1, 2, 3, 4, 5, 6, 7, 8, 9 or 10 and preferably having 0, 1, 2, 3, 4 or 5 amino acid insertions, deletions, substitutions or a combination thereof and wherein the second variable domain comprises a heavy chain variable region that comprises the amino acid sequence of MF8230 depicted in FIG. 8 (SEQ ID NO: 23) with 0-10 preferably 0-5 amino acid insertions, deletions, substitutions, additions or a combination thereof.

The mentioned at most 15, preferably 0, 1, 2, 3, 4, 5, 6, 7, 8, 9 or 10 and preferably 0, 1, 2, 3, 4 or 5 amino acid substitutions are preferably conservative amino acid substitutions, the insertions, deletions, substitutions or a combination thereof are preferably not in the CDR3 region of the VH chain, preferably not in the CDR1, CDR2 or CDR3 region of the VH chain and preferably not in the FR4 region.

Various methods are available to produce bispecific antibodies. One method involves the expression of two different heavy chains and two different light chains in a cell and collecting antibody that is produced by the cell. Antibody produced in this way will typically contain a collection of antibodies with different combinations of heavy and light chains, some of which are the desired bispecific antibody. The bispecific antibody can subsequently be purified from the collection. The ratio of bispecific to other antibodies that are produced by the cell can be increased in various ways. In a preferred embodiment, the ratio is increased by expressing not two different light chains but a common light chain in the cell. When a common light chain is expressed with the two different heavy chains, the ratio of bispecific antibody to other antibody that is produced by the cell is significantly improved over the expression of two different light chains. The ratio of bispecific antibody that is produced by the cell can be further improved by stimulating the pairing of two different heavy chains with each other over the pairing of two identical heavy chains. Methods and means are disclosed for producing bispecific antibodies (from a single cell), whereby means are provided that favor the formation of bispecific antibodies over the formation of monospecific antibodies. These methods can also be favorably employed in the present invention. Thus an invention as disclosed herein in one aspect provides a method for producing a bispecific antibody from a single cell, wherein said bispecific antibody comprises two CH3 domains that are capable of forming an interface, said method comprising providing in said cell a) a first nucleic acid molecule encoding a 1st CH3 domain comprising heavy chain, b) a second nucleic acid molecule encoding a 2nd CH3 domain comprising heavy chain, wherein said nucleic acid molecules are provided with means for preferential pairing of said 1st and 2nd CH3 domain comprising heavy chains, said method further comprising the step of culturing said host cell and allowing for expression of said two nucleic acid molecules and harvesting said bispecific antibody from the culture. Said first and second nucleic acid molecules may be part of the same nucleic acid molecule, vector or gene delivery vehicle and may be integrated at the same site of the host cell's genome. Alternatively, said first and second nucleic acid molecules are separately provided to said cell.

A preferred embodiment provides a method for producing a bispecific antibody according to an invention as disclosed herein from a single cell, wherein said bispecific antibody comprises two CH3 domains that are capable of forming an interface, said method comprising providing:
a cell having a) a first nucleic acid molecule encoding a heavy chain comprising an antigen binding site that binds EGFR and that contains a 1st CH3 domain, and b) a second nucleic acid molecule encoding a heavy chain comprising an antigen-binding site that binds ErbB-3 and that contains a 2nd CH3 domain, wherein said nucleic acid molecules are provided with means for preferential pairing of said 1st and 2nd CH3 domains,
said method further comprising the step of culturing said cell and allowing for expression of the proteins encoded by said two nucleic acid molecules and harvesting said bispecific IgG antibody from the culture. In a particularly preferred embodiment, said cell also has a third nucleic acid molecule encoding a common light chain. Said first, second and third nucleic acid molecule may be part of the same nucleic acid molecule, vector or gene delivery vehicle and may be integrated at the same site of the host cell's genome. Alternatively, said first, second and third nucleic acid molecules are separately provided to said cell. A preferred common light chain is based on O12, preferably it is the rearranged germline human kappa light chain IgV_{K}1 39*01/IGJ_{K}1*01, as described above. Means for preferential pairing of said 1st and said 2nd CH3 domain are preferably the corresponding mutations in the CH3 domain of the heavy chain coding regions. The preferred mutations to preferentially produce bispecific antibodies are the amino acid substitutions L351K and T366K (EU-numbering) in the first CH3 domain and the amino acid substitutions L351D and L368E in the second CH3 domain, or vice versa. Further provided is therefore a method according to an invention as disclosed herein for producing a bispecific antibody, wherein said first CH3 domain comprises the amino acid substitutions L351K and T366K (EU-numbering) and wherein said second

CH3 domain comprises the amino acid substitutions L351D and L368E, said method further comprising the step of culturing said cell and allowing for expression of proteins encoded by said nucleic acid molecules and harvesting said bispecific antibody from the culture. Also provided is a method according to an invention as disclosed herein for producing a bispecific antibody, wherein said first CH3 domain comprises the amino acid substitutions L351D and L368E (EU-numbering) and wherein said second CH3 domain comprises the amino acid substitutions L351K and T366K, said method further comprising the step of culturing said cell and allowing for expression of said nucleic acid molecules and harvesting said bispecific antibody from the culture. Antibodies that can 35 be produced by these methods are also part of the present invention. The CH3 hetero-dimerization domains are preferably IgG1 hetero-dimerization domains. The heavy chain constant regions comprising the CH3 hetero-dimerization domains are preferably IgG1 constant regions.

In one embodiment of the invention includes a nucleic acid molecule encoding an antibody heavy chain variable region. The nucleic acid molecule (typically an in vitro, isolated or recombinant nucleic acid molecule) preferably encodes a heavy chain variable region as depicted in FIG. 7 or FIG. 8, or a heavy chain variable region as depicted in FIG. 7 or FIG. 8 having 1, 2, 3, 4 or 5 amino acid insertions, deletions, substitutions or combination thereof. In a preferred embodiment the nucleic acid molecule comprises codon optimized nucleic acid sequence coding for an amino acid sequence as depicted in FIG. 7 or FIG. 8. The codon optimization is optimized for the species and/or the cell type of the antibody producing cell. For example, for CHO production the nucleic acid sequence of the molecule is codon optimized for Chinese hamster cells. The invention further provides a nucleic acid molecule encoding a heavy chain of FIG. 7 or FIG. 8.

A nucleic acid molecule as used in an invention as disclosed herein is typically but not exclusively a ribonucleic acid (RNA) or a deoxyribonucleic acid (DNA). Alternative nucleic acids are available for a person skilled in the art. A nucleic acid according to an invention as disclosed herein is for instance comprised in a cell. When said nucleic acid is expressed in said cell, said cell can produce an antibody according to an invention as disclosed herein. Therefore, in one embodiment of the invention includes a cell comprising an antibody according to invention as disclosed herein and/or a nucleic acid according to invention as disclosed herein. Said cell is preferably an animal cell, more preferably a mammal cell, more preferably a primate cell, most preferably a human cell. A suitable cell is any cell capable of comprising and preferably producing an antibody according to an invention as disclosed herein and/or a nucleic acid according to an invention as disclosed herein.

An invention as disclosed herein further provides a cell comprising an antibody according to an invention as disclosed herein. Preferably said cell (typically an in vitro, isolated or recombinant cell) produces said antibody. Said cell can also be a stored cell that is able to produce said antibody when taken out of storage and cultured. In a preferred embodiment said cell is a hybridoma cell, a Chinese hamster ovary (CHO) cell, an NSO cell or a PER-C6™ cell. In a particularly preferred embodiment said cell is a

CHO cell. Further provided is a cell culture comprising a cell according to an invention as disclosed herein. Various institutions and companies have developed cell lines for the large scale production of antibodies, for instance for clinical use. Non-limiting examples of such cell lines are CHO cells, NSO cells or PER.C6™ cells. These cells are also used for other purposes such as the production of proteins. Cell lines developed for industrial scale production of proteins and antibodies are herein further referred to as industrial cell lines. Thus a preferred embodiment includes use of a cell line developed for the large scale production of antibody for the production of an antibody of an invention as disclosed herein, including preferably a cell for producing an antibody comprising a nucleic acid molecule that codes for a VH, a VL, and/or a heavy chain as depicted in FIG. 7 or FIG. 8.

The invention further provides a method for producing an antibody comprising culturing a cell of an invention as disclosed herein and harvesting said antibody from said culture. Preferably said cell is cultured in a serum free medium. Preferably said cell is adapted for suspension growth. Further provided is an antibody obtainable by a method for producing an antibody according to an invention as disclosed herein. The antibody is preferably purified from the medium of the culture. Preferably said antibody is affinity purified.

A cell of an invention as disclosed herein is for instance a hybridoma cell line, a CHO cell, a 293F cell, an NSO cell or another cell type known for its suitability for antibody production for clinical purposes. In a particularly preferred embodiment said cell is a human cell. Preferably a cell that is transformed by an adenovirus El region or a functional equivalent thereof. A preferred example of such a cell line is the PER.C6TM cell line or equivalent thereof. In a particularly preferred embodiment said cell is a CHO cell or a variant thereof. Preferably a variant that makes use of a Glutamine synthetase (GS) vector system for expression of an antibody.

Antibodies of an invention as disclosed herein can be produced at levels >50 mg/L after transient transfection in suspension 293F cells. The bispecific antibodies can be purified to greater than 98% purity with yields >70%. Analytical characterization studies show bispecific lgG1 antibody profiles that are comparable to bivalent monospecific lgG1. In terms of functional activity a bispecific antibody of an invention as disclosed herein can demonstrate superior potency compared to cetuximab in vitro and in vivo.

The invention further provides a pharmaceutical composition comprising an antibody according to an invention as disclosed herein. The pharmaceutical composition preferably comprises a preferably pharmaceutically acceptable excipient or carrier.

An antibody can comprise a label, preferably a label for in vivo imaging. Such a label is typically not necessary for therapeutic applications. In for instance a diagnostic setting, a label can be helpful. For instance in visualizing target cells in the body. Various labels are suited and many are well known in the art. In a preferred embodiment the label is a radioactive label for detection. In another preferred embodiment, the label is an infrared label. Preferably the infrared label is suited for in vivo imaging. Various infrared labels are available to the person skilled in the art.

Preferred infrared labels are for instance, IRDye 800; IRDye 680RD; IRDye 680LT; IRDye 750; IRDye 700DX; IRDye 800RS IRDye 650; IRDye 700 phosphoramidite; IRDye 800 phosphoramidite (LI-COR USA; 4647 Superior Street; Lincoln, Nebraska).

The invention further provides a method for the treatment of a subject that has a tumor or is at risk of having said tumor comprising administering to the subject in need thereof an antibody or pharmaceutical composition according to an invention as disclosed herein. The tumor is preferably an EGFR, cMET or EGFR/cMET positive tumor. Before start of said treatment, the method preferably further comprises determining whether said subject has such an EGFR, cMET or EGFR/cMET positive tumor. The invention further provides an antibody or pharmaceutical composition of an invention as disclosed herein for use in the treatment of a subject that has or is at risk of having an EGFR, cMET or EGFR/cMET positive tumor.

To establish whether a tumor is positive for EGFR the skilled person can for instance determine the EGFR amplification and/or immuno-histochemistry staining At least 10% of the tumor cells in a biopsy should be positive. The biopsy can also contain 20%, 30% 40% 50% 60% 70% or more positive cells. To establish whether a tumor is positive for cMET the skilled person can for instance determine the cMET amplification and/or staining in immunohistochemistry. At least 10% of the tumor cells in a biopsy should be positive. The biopsy can also contain 20%, 30% 40% 50% 60% 70% or more positive cells.

The invention as disclosed herein can be applied to a wide range of cancers, like breast cancer, colon cancer, pancreatic cancer, gastric cancer, ovarian cancer, colorectal cancer, head- and neck cancer, lung cancer including non-small cell lung cancer, bladder cancer and the like. The tumor may be an EGFR, cMET or EGFR/cMET positive cancer. An embodiment of the invention may preferably treat a positive cancer that is a breast cancer, such as early-stage breast cancer. In another embodiment of the invention may preferably treat the EGFR, cMET or EGFR/cMET positive cancer that is colorectal cancer. The invention as disclosed herein can be applied to a wide range of EGFR, cMET or EGFR/cMET positive cancers, like breast cancer, colon cancer, pancreatic cancer, gastric cancer, ovarian cancer, colorectal cancer, head- and neck cancer, lung cancer including non-small cell lung cancer, bladder cancer and the like. The subject is preferably a human subject. The subject is preferably a subject eligible for antibody therapy using an EGFR specific antibody such as cetuximab. In a preferred embodiment the invention may preferably treat a subject that comprises a tumor, preferably an EGFR/cMET positive cancer, preferably a tumor/cancer with an EGFR RTK resistant phenotype, an EGFR monoclonal antibody resistant phenotype or a combination thereof.

The amount of antibody to be administered to a patient is typically in the therapeutic window, meaning that a sufficient quantity is used for obtaining a therapeutic effect, while the amount does not exceed a threshold value leading to an unacceptable extent of side-effects. The lower the amount of antibody needed for obtaining a desired therapeutic effect, the larger the therapeutic window will typically be. An antibody according to an invention as disclosed herein exerting sufficient therapeutic effects at low dosage is, therefore, preferred. The dosage can be in range of the dosing regimen of cetuximab. The dosage can also be lower.

A bispecific antibody according to an invention as disclosed herein preferably induces less skin toxicity as compared to cetuximab under otherwise similar conditions. A bispecific antibody according to an invention as disclosed herein preferably produces less proinflammatory chemokines, preferably of CXCL14 as compared to cetuximab under otherwise similar conditions. A bispecific antibody according to an invention as disclosed herein preferably induces less impairment of antimicrobial RNAses, preferably Rnase 7, as compared to cetuximab under otherwise similar conditions.

The present invention describes among others antibodies that target the EGFR and cMET receptors and result in potent proliferation inhibition of cancer cell lines in vitro and tumor growth inhibition in vivo. A bispecific antibody of an invention as disclosed herein can combine low toxicity profiles with high efficacy. An antibody of invention as disclosed herein can be useful in various types and lines of EGFR-targeted therapies. An antibody of an invention as disclosed herein can have an increased therapeutic window when compared to an antibody that binds the same antigen(s) with both arms. A bispecific antibody of an invention as disclosed herein can exhibit better growth inhibitory effects in vitro, in vivo or a combination thereof when compared to the cetuximab antibody.

The invention also provides a bispecific antibody of an invention as disclosed herein, for use in the treatment of subject that may have one or more of a variety of different kinds of tumors. The tumor may be an EGFR positive tumor, a cMET positive tumor or an EGFR and cMET positive tumor. The tumor may be a breast cancer; colon cancer, pancreatic cancer, gastric cancer, ovarian cancer, colorectal cancer, head- and neck cancer, lung cancer including non-small cell lung cancer or bladder cancer. The tumor may be resistant to treatment with an EGFR tyrosine kinase inhibitor. The EGFR tyrosine kinase inhibitor is preferably erlotinib, gefitinib, or afatinib, an analogue of erlotinib, gefitinib or afatinib or a combination of one or more of the respective compounds and/or analogues thereof. The treatment preferably further comprises treatment with an EGFR tyrosine kinase inhibitor. When co-treating with an EGFR tyrosine kinase inhibitor the tumor can be resistant to the treatment with the EGFR tyrosine kinase inhibitor. The co-treatment at least partly restores sensitivity of the tumor to the tyrosine kinase inhibitor. The EGFR tyrosine kinase inhibitor can be a first generation EGFR tyrosine kinase inhibitor. Examples of clinically relevant first generation EGFR tyrosine kinase inhibitors are erlotinib and gefitinib. In this and other embodiments the tumor may be an HGF-associated tumor.

An EGFR-positive tumor is typically a tumor that has an EGFR activating mutation. An EGFR activating mutation is a mutation of EGFR that results in activation of the EGF/EGFR signaling pathway. The EGFR activating mutation may be important for a cancerous state of the tumor. One of the ways in which such tumors can become insensitive to EGFR targeted therapy is by activation of the HGF/cMET signaling pathway. The tumor may be an HGF-associated tumor. Activation of the cMET/HGF signaling pathway is one of the ways in which an EGFR-positive tumor can escape treatment with an EGFR-targeted therapy. The cMET/HGF pathway can be activated in various ways. Various methods of activation are described in the art some of which are detailed herein. An antibody of an invention as disclosed herein is particularly suited for the treatment of tumors wherein activation of the cMET/HGF signaling pathway is associated with the presence of or excess of HGF. Such cMET positive tumors are referred to as HGF-associated tumors or HGF-dependent tumors. An antibody of an invention as disclosed herein can also be used to at least in part inhibit this possible escape mechanism of EGFR positive tumors. Such tumors can escape EGFR-targeted therapy through the selected outgrowth of tumor cells wherein, in addition, the cMET/HGF signaling pathway is activated. Such cells may be present at the start of the EGFR-targeted therapy. Such cells have a selective growth advantage over HGF/cMET signaling negative tumor cells. The tumor may be a tumor wherein the HGF/cMET signaling pathway is activated. The tumor may be a tumor that is associated with elevated levels of hepatocyte growth factor (HGF) or overexpression of the HGF receptor c-Met. The tumor may be a tumor wherein growth is driven by the

EGF and/or HGF. A tumor is said to be driven by a certain growth factor if the signaling pathway is activated in cells of the tumor in response to the presence of the growth factor and removal of the growth factor results in inhibition of the growth of the cells of the tumor. Reduction can be measured by reduced cell division and/or induced cell kill such as apoptosis. A tumor is an HGF-associated tumor if under conditions that would otherwise be permissive for the growth of the tumor, the tumor growths or growths faster in the presence of HGF.

EGFR-targeted therapies for various tumors have been reviewed by Vecchione et al., EGFR-targeted therapy.” Experimental cell research Vol 317 (2011): 2765-2771. In general EGFR-targeted therapy is a therapy with a molecule that interacts with EGFR and inhibits EGFR-mediated signaling in the cell.

The method of treatment or antibody for use in the treatment as indicated herein preferably further comprises the step of determining whether the tumor is an HGF-associated tumor.

An antibody of an invention as disclosed herein can inhibit growth of an HGF-associated tumor.

In some embodiments wherein a cMET binding variable domain is described to have a CDR2 sequence “WINTYTGDPTYAQGFTG” (SEQ ID NO: 29) the CDR2 sequence can also be “WINTYTGDPTYAQGFT” (SEQ ID NO: 45).

Where herein ranges are given as between number 1 and number 2, the range includes the number 1 and number 2. For instance a range of between 2-5 includes the number 2 and 5.

When herein reference is made to an affinity that is higher than another, the Kd =lower than the other Kd. For the avoidance of doubt a Kd of 10e−9 M is lower than a Kd of 10e−8 M. The affinity of an antibody with a Kd of 10e−9 M for a target is higher than when the Kd is 10e−8 M.

A reference herein to a patent document or other matter which is cited is not to be taken as an admission that that document or matter was known or that the information it contains was part of the common general knowledge as at the priority date of any of the claims.

For the purpose of clarity and a concise description, features are described herein as part of the same or separate embodiments, however, it will be appreciated that the scope of the invention as disclosed herein may include embodiments having combinations of all or some of the features described.

### BRIEF DESCRIPTION OF THE DRAWINGS

FIG. 1. Amino acid sequence of the heavy chain variable regions of variable domains referred to in this application.

FIG. 2. Functionality of anti-EGFR cLC bivalent antibodies in inhibiting the EGF induced death of A431 cells. The Y axis (counts) shows the fluorescence readout of the assay, reminiscent of the number of metabolically active cells, as a function of the concentration of antibody used (X-axis). PG3370 is able to inhibit EGF-induced cell death and therefore shows enhanced growth of the cells with increasing antibody concentration. Molecules having variable region amino acid sequences of cetuximab/Erbitux, referred to herein as cetuximab or reference antibody cetuximab, were used in experiments as an internal standard (black dots).

FIG. 3. The effect of cMET x EGFR bispecifics on wound healing in H385 cells (panel A) and EBC-1 cells (panel B).
Cells were incubated either without (mock) or with 12.5 ng/ml EGF or 15 ng/ml HGF or a combination of HGF and EGF (15 ng/ml and 12.5 ng/ml) with addition of 5 individual cMETxEGFR bispecifics. As a control cetuximab in combination with 2994 Fab was included. The Y-axis depicts the percentage of wound closure measured by time-lapse microscopy.

FIG. 4. FACS analysis on the EGFR and cMET expression analysis in the TKI resistant NSCLC cells, HCC827 and PC-9 cells.
(A) Both cells lines were characterized for the expression of EGFR (x-axis) and cMET (y-axis) using fluorescently labeled antibodies. All HCC827 cells show EGFR expression and can be subdivided into a EGFR^{high}, cMET^{pos} population and a EGFR^{pos}, cMET^{neg} population. PC-9 cells contain a small population of EGFR^{high} and cMET^{pos} cells and a minimal population of EGFR^{pos} and cMET^{neg} cells.
(B) Graph representing the distribution of the different cell populations in PC-9 and HCC827 cells.

FIG. 5. Example of the effect of PB8532 and PB8388 on HGF induced resistance to TKI inhibitors in PC-9 (panel A) and HCC827 (panel B) cells.
Cells were pre-treated with bispecific PB8532, PB8388, or the cetuximab/5D5 Fab mixture and incubated with HGF and/or EGF in combination with a TKI inhibitor, after which the proliferation was measured. PB8532 inhibits HGF mediated and EGF mediated gefitinib resistance in PC-9 cells and HCC827 cells.

FIG. 6. Effect of treatment with the indicated antibodies on HGF induced cMET phosphorylation or EGF induced EGFR phosphorylation on PC-9 and HCC827 cells. Antibodies (100 nM) were incubated for 15 minutes at 37° C. where after cell extracts were generated and applied to Western Blot analysis for detection of (p)EGFR and (p)cMET. Anti-vinculin antibody was included as a protein loading control.

FIG. 7. MF3370 and variants thereof. The CDR1, CDR2 and CDR3 sequences in MF8226 are underlined from left to right. The CDRs in the other sequences are at the corresponding positions.

FIG. 8. MF4356 and variants thereof. The CDR1, CDR2 and CDR3 sequences in MF4356 are underlined from left to right. The CDRs in the other sequences are at the corresponding positions.

FIG. 9. Common light chain used in mono- and bispecific IgG.

FIG. 9A: Common light chain amino acid sequence. FIG. 9B: Common light chain variable domain DNA sequence and translation (IGKV1-39/jk1). FIG. 9C: Common light chain constant region DNA sequence and translation. FIG. 9D: IGKV1-39/jk5 common light chain variable domain translation. FIG. 9E: V-region IGKV1-39A.

FIG. 10. IgG heavy chains for the generation of bispecific molecules. FIG. 10A: CH1 region. FIG. 10B: hinge region. FIG. C: CH2 region. FIG. 10D: CH2 containing L235G and G236R silencing substitutions. FIG. 10E: CH3 domain containing substitutions L351K and T366K (KK). FIG. 10F; CH3 domain containing substitutions L351D and L368E (DE).

FIG. 11. Inhibition of EGF binding to recombinant EGFR in ELISA. Biotinylated EGF was allowed to bind coated EGFR in the presence of a serial dilution of IgG. Cetuximab was used as a positive control and PG2708 as a negative control antibody (Neg ctrl Ab). EGF binding was detected by streptavidin HRP.

FIG. 12. Determination of cynomolgus EGFR cross reactivity by FACS analysis. CHO-K1 cells were transfected with human EGFR or cynomolgus EGFR constructs. Antibodies were allowed to bind the transfected cells and CHO-K1 cells at 5 μg/ml. Cetuximab was used as a positive control and PG2708 as a negative control antibody (Neg ctrl Ab). Bound antibodies were detected by a PE conjugated antibody.

FIG. 13. Determination of mouse EGFR and cMET cross reactivity by ELISA. Upper panel; A fixed concentration of antibody (5 μg/ml) was tested in a serial titration in microtiter plates coated with mouse EGFR and human EGFR. Anti-EGFR antibodies and PG2708 (neg Ctrl Ab) were allowed to bind and detected by an HRP conjugated antibody. Lower panel; a serial titration of antibodies was allowed to bind coated human and mouse cMET. The human/mouse cross reactive antibody BAF527 was included as a positive control antibody and PG2708 was added as a negative control antibody (Neg Ctrl Ab). Bound antibodies were detected by streptavidin HRP.

FIG. 14. Inhibition of ligand dependent N87 proliferation. A serial antibody titration was incubated with N87 cells in the presence of HGF(A), EGF (B) or EGF/HGF (C). Cell proliferation was measured by Alamar Blue. Fab 5D5/cetuximab in an equimolar concentration was included as a positive control antibody. The Y-axis represents the fluorescence intensity as an indicator of cell proliferation. The X-axis represents the different concentration of the tested antibodies.

FIG. 15. An example of ADCC activity of cMETxEGFR bispecific antibodies in N87 cells (A) and MKN-45 cells (B) using the high affinity FcγRIIIa ADCC reporter assay. The X-axis represents the added antibody concentration. The Y-Axis represents the Luminescence (RLU) as a read out for ADCC activity. Anti-EGFR antibody cetuximab was included as a positive control antibody.

FIG. 16. The effect of HGF on the efficacy of the TKIs erlotinib and gefitinib in PC-9 (A) and HCC827 (B) cells. Cells were incubated with increasing concentrations of HGF (0 to 120 ng/mL) in combination with 300 nM erlotinib or gefitinib, after which cell proliferation was measured. In both cell lines HGF induced a dose-dependent resistance to the TKIs.

FIG. 17. Testing of affinity binding of ADCC-enhanced c-MET x EGFR variants. CHO-Kl cells stably expressing EGFR (A) or MKN-45 cells endogenously expressing c-MET (B) were incubated at 2×10⁵ cells/well with increasing concentrations of antibody as indicated. After washing, binding was detected with anti-human IgG-PE (3 μg/ml). Stained cells were analyzed on an iQue system and mean fluorescence intensity (MFI) was calculated. Control antibodies were MF1337xMF1337; TTxTT negative control; dark triangles at the bottom) and MF4356xMF3770 (PB8532p04; c-METxEGFR positive control for c-MET; black triangles). TT stands for tetanus toxoid. ADCC indicates antibodies with enhanced ADCC function through co-transfection with DNA encoding the RMD enzyme to remove a fucose residue from the Fc region of IgG1.

FIG. 18. Results of ADCC reporter assay to confirm enhanced ADCC effector function. EGFR-expressing BxPC-3 cells (left) or c-MET-expressing MKN-45 cells (right) were mixed with ADCC effector cells at an E:T ratio of 15:1,and incubated in the presence of a titration of test antibody (0.01 to 10 μg/ml). After 6 hours, Bio-Glo reagent was added and luminescence measured using a microplate reader. The greater the level of luminescence, the greater the degree of interaction between target and effector cells induced by the test antibody. Top panels show results of high-affinity assay and bottom panels those of low-affinity assay. The negative control antibody was PG1337p218 (anti-TT, light triangles at the bottom); the other control antibodies were 3178x4280 (HER3 x EGFR, ADCC-enhanced, light closed circles (at the top in the left hand top panel); 3178x4280 (HER3 x EGFR, non-ADCC-enhanced, black crosses, at the bottom); 4356x3370 (c-MET x EGFR, ADCC-enhanced, open light circles); 3370x4356 (EGFR x c-MET, non-ADCC-enhanced, black crosses and dashed lines); and Cetuximab (anti-EGFR, small black circles).

FIG. 19. Erlotinib induces an anti-tumor response in NGS-hHGFki mice engrafted with HCC827 cells as long as mice receive treatment. Black arrow indicates start of treatment.

FIG. 20. PB8532 alone and in combination with erlotinib induces an anti-tumor response in NGS-hHGFki mice engrafted with HCC827 cells. Black arrow indicates start of treatment; grey arrows in the X-axis indicate weekly antibody treatments.

FIG. 21. The anti-tumor response induced by PB8532 alone and in combination with erlotinib is superior to that of erlotinib, even after treatment stops. Black arrow indicates start of treatment; grey arrows in the X-axis indicate weekly antibody treatments.

FIG. 22. The anti-tumor response induced by PB8532 alone and in combination with erlotinib is superior to that of erlotinib. Black arrow indicates start of treatment; grey arrows in the X-axis indicate weekly antibody treatments. Treatment with the cMET antibody LY2875358 with and without erlotinib treatment was less effective than PB8532 even without erlotinib treatment.

FIG. 23. The anti-tumor response induced by the cMETxEGFR bispecific antibody PB19478 is effective also when the tumor develops resistance to erlotinib. Black arrows indicate the start of the erlotinib treatment and the start of the PB19478 treatment.

### EXAMPLES

As used herein “MFXXXX” wherein X is independently a numeral 0-9, refers to a Fab comprising a variable domain wherein the VH has the amino acid sequence identified by the 4 digits. Unless otherwise indicated the light chain variable region of the variable domain typically has a sequence of FIG. 9A, typically 9B. “MFXXXX VH” refers to the amino acid sequence of the VH identified by the 4 digits. The MF further comprises a constant region of a light chain and a constant region of a heavy chain that normally interacts with a constant region of a light chain. PG refers to a monospecific antibody comprising identical heavy and light chains. PB refers to a bispecific antibody with two different heavy chains. The VH variable regions of the heavy chains differ and typically also the CH3 region, wherein one of the heavy chains has a KK mutation of its

CH3 domain and the other has the complementing DE mutation of its CH3 domain (see for reference PCT/NL2013/050294 (published as WO2013/157954).

### Example 1: Materials and Methods

### Cell Lines:

EBC-1 [JCRB0820], PC-9 [RCB0446], H358 [ATCC® CRL-5807™], HCC827 [ATCC® CRL-2868™],MKN-45 [DSMZ ACC 409] N87 [ATCC®CRL-5822™] and A431 [ATCC® CRL-1555™] cell lines were purchased and routinely maintained in growth media supplemented with 10% heat inactivated fetal bovine serum (FBS). HEK293F Freestyle cells were obtained from Invitrogen and routinely maintained in 293 FreeStyle medium.

cDNA Constructs:

### Generation of cMET and EGFR Expression Vectors for Generation of Stable Cell Lines (cMET and EGFR) and for Immunization (cMET)

Full length cDNA of each target including unique restriction sites for cloning and kozak consensus sequence for efficient translation was either synthetized, or obtained via PCR amplification on a commercially available expression construct, containing the target cDNA, with specific primers that introduced unique restriction sites for cloning and kozak consensus sequence for efficient translation. The full length cDNA of each target was cloned into a eukaryotic expression construct such as pcDNA3.1, whereas the extracellular domains were cloned into pVAX₁ and pDisplay. The insert sequences were verified by comparison with NCBI Reference amino acid sequences.

Amino acid sequence full length human EGFR insert for expression on the cell surface (Identical to GenBank: NP_00533):

(SEQ ID NO: 42)
MRPSGTAGAALLALLAALCPASRALEEKKVCQGTSNKLTQLGTFEDHFLS
LQRMFNNCEVVLGNLEITYVQRNYDLSFLKTIQEVAGYVLIALNTVERIP
LENLQIIRGNMYYENSYALAVLSNYDANKTGLKELPMRNLQEILHGAVRF
SNNPALCNVESIQWRDIVSSDFLSNMSMDFQNHLGSCQKCDPSCPNGSCW
GAGEENCQKLTKIICAQQCSGRCRGKSPSDCCHNQCAAGCTGPRESDCLV
CRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDSLS
INATNIKHFKNCTSISGDLHILPVAFRGDSFTHTPPLDPQELDILKTVKE
ITGFLLIQAWPENRTDLHAFENLEIIRGRTKQHGQFSLAVVSLNITSLGL
RSLKEISDGDVIISGNKNLCYANTINWKKLFGTSGQKTKIISNRGENSCK
ATGQVCHALCSPEGCWGPEPRDCVSCRNVSRGRECVDKCNLLEGEPREFV
ENSECIQCHPECLPQAMNITCTGRGPDNCIQCAHYIDGPHCVKTCPAGVM
GENNTLVWKYADAGHVCHPLCHNCTYGCTGPGLEGCPTNGPKIPSIATGM
VGALLLLLVVALGIGLFMRRRHIVRKRTLRRLLQERELVEPLTPSGEAPN
QALLRILKETEFKKIKVLGSGAFGTVYKGLWIPEGEKVKIPVAIKELREA
TSPKANKEILDEAYVMASVDNPHVCRLLGICLTSTVQLITQLMPFGCLLD
YVREHKDNIGSQYLLNWCVQIAKGMNYLEDRRLVHRDLAARNVLVKTPQH
VKITDFGLAKLLGAEEKEYHAEGGKVPIKWMALESILHRIYTHQSDVWSY
GVTVWELMTFGSKPYDGIPASEISSILEKGERLPQPPICTIDVYMIMVKC
WMIDADSRPKFRELIIEFSKMARDPQRYLVIQGDERMHLPSPTDSNFYRA
LMDEEDMDDVVDADEYLIPQQGFFSSPSTSRTPLLSSLSATSNNSTVACI
DRNGLQSCPIKEDSFLQRYSSDPTGALTEDSIDDTFLPVPEYINQSVPKR
PAGSVQNPVYHNQPLNPAPSRDPHYQDPHSTAVGNPEYLNTVQPTCVNST
FDSPAHWAQKGSHQISLDNPDYQQDFFPKEAKPNGIFKGSTAENAEYLRV
APQSSEFIGA

Of which:

(SEQ ID NO: 141)
MRPSGTAGAALLALLAALCPASR: signal peptide.
(SEQ ID NO: 142)
ALEEKKVCQGTSNKLTQLGTFEDHFLSLQRMFNNCEVVLGNEITYVQRNY
DLSFLKTIQEVAGYVLIALNTVERIPLENLQIIRGNMYYENSYALAVLSN
YDANKTGLKELPMRNLQEILHGAVRFSNNPALCNVESIQWRDIVSSDFLS
NMSMDFQNHLGSCQKCDPSCPNGSCWGAGEENCQKLTKIICAQQCSGRCR
GKSPSDCCHNQCAAGCTGPRESDCLVCRKFRDEATCKDTCPPLMLYNPTT
YQMDVNPEGKYSFGATCVKKCPRNYVVTDHGSCVRACGADSYEMEEDGVR
KCKKCEGPCRKVCNGIGIGEFKDSLSINATNIKHPKNCTSISGDLHILPV
AFRGDSFTHTPPLDPQELDILKTVKEITGFLLIQAWPENRTDLHAFENLE
IIRGRTKQHGQFSLAVVSLNITSLGLRSLKEISDGDVIISGNKNLCYANT
INWKKLFSTSGQKTKIISNRGENSCKATGQVCHALCSPEGCWGPEPRDCV
SCRNVSRGRECVDKCNLLEGEPREFVENSECIQCHPECLPQAMNITCTGR
GPDNCIQCAHYIDGPHCVKTCPAGVMGENNTLVWKYADAGHVCHLCHPNC
TYGCTGPGLEGCPTNGPKIPS: ECD of human EGFR.
(SEQ ID NO: 143)
IATGMVGALLLLLVVALGIGLFM: predicted TM region.
(SEQ ID NO: 144)
RRRHIVRKRTLRRLLQERELVEPLTPSGEAPNQALLRILKETEFKKIKVL
GSGAFGTVYKGLWIPEGEKVKIPVAIKELREATSPKANKEILDEAYVMAS
VDNPHVCRLLGICLTSTVQLITQLMPFGCLLDYVREHKDNIGSQYLLNWC
VQIAKGMNYLEDRRLVHRDLAARNVLVKTPQHVKITDFGLAKLLGAEEKE
YHAEGGKVPIKWMALESILHRIYTHQSDVWSYGVTVWELMTFGSKPYDGI
PASEISSILEKGERLPQPPICTIDVYMIMVKCWMIDADSRPKFRELIIEF
SKMARDPQRYLVIQGDERMHLPSPTDSNFYRALMDEEDMDDVVDADEYLI
PQQGFFSSPSTSRTPLLSSLSATSNNSTVACIDRNGLQSCPIKEDSFLQR
YSSDPTGALTEDSIDDTFLPVPEYINQSVPKRPAGSVQNPVYHNQPLNPA
PSRDPHYQDPHSTAVGNPEYLNTVQPTCVNSTFDSPAHWAQKGSHQISLD
NPDYQQDFFPKEAKPNGIFKGSTAENAEYLRVAPQSSEFIGA:
intracellular tail.

Amino acid sequence of extracellular domain of human EGFRvarIII a natural occurring EGFR variant VAR_066493 [Ji H., Zhao X; PNAS 103:7817-7822(2006)] caused by an in-frame deletion of exons 2-7. The_below indicates the location lacking amino acids 30-297

(SEQ ID NO: 43)
MRPSGTAGAALLALLAALCPASRALEEKK_GNYVVTDHGSCVRACGADSY
EMEEDGVRKCKKCEGPCRKVCNGIGIGEFKDSLSINATNIKHFKNCTSIS
GDLHILPVAFRGDSFTHTPPLDPQELDILKTVKEITGFLLIQAWPENRTD
LHAFENLEIIRGRTKQHGQFSLAVVSLNITSLGLRSLKEISDGDVIISGN
KNLCYANTINWKKLFGTSGQKTKIISNRGENSCKATGQVCHALCSPEGCW
GPEPRDCVSCRNVSRGRECVDKCNLLEGEPREFVENSECIQCHPECLPQA
MNITCTGRGPDNCIQCAHYIDGPHCVKTCPAGVMGENNTLVWKYADAGHV
CHLCHPNCTYGCTGPGLEGCPTNGPKIPS

Of which:

(SEQ ID NO: 145)
MRPSGTAGAALLALLAALCPASR: signal peptide.
(SEQ ID NO: 146)
ALEEKK_GNYVVTDHGSCVRACGADSYEMEEDGVRKCKKCEGPCRKVCNG
IGIGEFKDSLSINATNIKHFKNCTSISGDLHILPVAFRGDSFTHTPPLDP
QELDILKTVKEITGFLLIQAWPENRTDLHAFENLEIIRGRTKQHGQFSLA
VVSLNITSLGLRSLKEISDGDVIISGNKNLCYANTINWKKLFGTSGQKTK
IISNRGENSCKATGQVCHALCSPEGCWGPEPRDCVSCRNVSRGRECVDKC
NLLEGEPREFVENSECIQCHPECLPQAMNITCTGRGPDNCIQCAHYIDGP
HCVKTCPAGVMGENNTLVWKYADAGHVCHLCHPNCTYGCTGPGLEGCPTN
GPKIPS: ECD of EGFRvarIII

Amino acid sequence chimeric macaque (Macaca mulatta) extra cellular EGFR domain hybrid with human EGFR transmembrane and intracellular domain for expression on the cell surface (Identical to GenBank: XP_014988922.1. Human EGFR sequence underlined in the example below.

(SEQ ID NO: 44)
MGPSGTAGAALLALLAALCPASRALEEKKVCQGTSNKLTQLGTFEDHFLS
LQRMFNNCEVVLGNLEITYVQRNYDLSFLKTIQEVAGYVLIALNTVERIP
LENLQIIRGNMYYENSYALAVLSNYDANKTGLKELPMRNLQEILHGAVRF
SNNPALCNVESIQWRDIVSSEFLSNMSMDFQNHLGSCQKCDPSCPNGSCW
GAGEENCQKLTKIICAQQCSGRCRGKSPSDCCHNQCAAGCTGPRESDCLV
CRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDTLS
INATNIKHFKNCTSISGDLHILPVAFRGDSFTHTPPLDPQELDILKTVKE
ITGFLLIQAWPENRTDLHAFENLEIIRGRTKQHGQFSLAVVSLNITSLGL
RSLKEISDGDVIISGNKNLCYANTINWKKLFGTSSQKTKIISNRGENSCK
ATGQVCHALCSPEGCWGPEPRDCVSCQNVSPGRECVDKCNILEGEPREFV
ENSECIQCHPECLPQVMNITCTGRGPDNCIQCAHYIDGPHCVKTCPAGVM
GENNTLVWKYADAGHVCHLCHPNCTYGCTGPGLEGCARNGPKIPSIATGM
LGALLLLLVVALGIGLFMRRRHIVRKRTLRRLLQERELVEPLTPSGEAPN
QALLRILKETEFKKIKVLGSGAFGTVYKGLWIPEGEKVKIPVAIKELREA
TSPKANKEILDEAYVNASVDNPHVCRLLGICLTSTVQLITQLMPFGCLLD
YVREHKDNIGSQYLLNWCVQIAKGMNYLEDRRLVHRDLAARNVLVKTPQH
VKITDFGLAKLLGAEEKEYHAEGGKVPIKWMALESILHRIYTHQSDVWSY
GVTVWELMTFGSKPYDGIPASEISSILEKGERLPQPPICTIDVYMIMVKC
WMIDADSRPKFRELIIEFSKMARDPQRYLVIQGDERMHLPSPTDSNFYRA
LMDEEDMDDVVDADEYLIPQQGFFSSPSTSRTPLLSSLSATSNNSTVACI
DRNGLQSCPIKEDSFLQRYSSDPTGALTEDSIDDTFLPVPEYINQSVPKR
PAGSVQNPVYHNQPLNPAPSRDPHYQDPHSTAVGNPEYLNTVQPTCVNST
FDSPAHWAQKGSHQISLDNPDYQQDFFPKEAKPNGIFKGSTAENAEYLRV
APQSSEFIGA

Of which:

(SEQ ID NO: 147)
MGPSGTAGAALLALLAALCPASR: signal peptide.
(SEQ ID NO: 148)
LEEKKVCQGTSNKLTQLGTFEDHFLSLQRMFNNCEVVLGNLEITYVQRNY
DLSFLKTIQEVAGYVLIALNTVERIPLENLQIIRGNMYYENSYALAVLSN
YDANKTGLKELPMRNLQEILHGAVRFSNNPALCNVESIQWRDIVSSDFLS
NMSMDFQNHLGSCQKCDPSCPNGSCWGAGEENCQKLTKIICAQQCSGRCR
GKSPSDCCHNQCAAGCTGPRESDCLVCRKFRDEATCKDTCPPLMLYNPTT
YQMDVNPEGKYSFGATCVKKCPRNYVVTDHGSCVRACGADSYEMEEDGVR
KCKKCEGPCRKVCNGIGIGEFKDSLSINATNIKHFKNCTSISGDLHILPV
AFRGDSFTHTPPLDPQELDILKTVKEITGFLLIQAWPENRTDLHAFENLE
IIRGRTKQHGQFSLAVVSLNITSLGLRSLKEISDGDVIISGNKNLCYANT
INWKKLFGTSGQKTKIISNRGENSCKATGQVCHALCSPEGCWGPEPRDCV
SCRNVSRGRECVDKCNLLEGEPREFVENSECIQCHPECLPQAMNITCTGR
GPDNCIQCAHYIDGPHCVKTGPAGVMGENNTLVWKYADAGHVCHLCHPNC
TYGCTGPGLEGCPTNGPKIPS: ECD of cyEGFR

Amino acid sequence full length human cMET insert for expression on the cell surface (Identical to GenBank: P08581-2). The sequence differs from the reference sequence at position with an insertion at 755-755

(SEQ ID NO: 46)
S→STWWKEPLNIVSFLFCFASMKAPAVLAPGILVLLFTLVQRSNGECKEA
LAKSEMNVNMKYQLPNFTAETPIQNVILHEHHIFLGATNYIYVLNEEDLQ
KVAEYKTGPVLEHPDCFPCQDCSSKANLSGGVWKDNINMALVVDTYYDDQ
LISCGSVNRGTCQRHVFPHNHTADIQSEVHCIFSPQIEEPSQCPDCVVSA
LGAKVLSSVKDRFINFFVGNTINSSYFPDHPLHSIDVRRLKETKDGFMFL
TDQSYIDVLPEFRDSYPIKYVHAFESNNFIYFLTVQRETLDAQTFHTRII
RFCSINSGLHSYMEMPLECILTEKRKKRSTKKEVFNILQAAYVSKPGAQL
ARQIGASLNDDILFGVFAQSKPDSAEPMDRSAMCAFPIHYVNDFFNKIVN
KNNVRCLQHFYGPNHEHCFNRTLLRNSSGCEARRDEYRTEFTTALQRVDL
FMGQFSEVLLTSISTFIKGDLTIANLGTSEGRFMQVVVSRSGPSTPHVNF
LLDSHPVSPEVIVEHTLNQNGYTLVITGKKITKIPLNGLGCRHFQSCSQC
LSAPPFVQCGWCHDKCVRSEECLSGTWTQQICLPAIYKVFPNSAPLEGGT
RLTICGWDFGFRRNNKFDLKKTRVLLGNESCTLTLSESTMNTLKCTVGPA
MNKHFNMSIIISNGHGTTQYSTFSYVDPVITSISPKYGPMAGGTLLTLTG
NYLNSGNSRHISIGGKTCTLKSVSNSILECYTPAQTISTEFAVKLKIDLA
NRETSIFSYREDPIVYEIHPTKSFISTWWKEPLNIVSFLFCFASGGSTIT
GVGKNLNSVSVPRMVINVHEAGRNFTVACQHRSNSEIICCTTPSLQQLNL
QLPLKTKAFFMLDGILSKYFDLIYVHNPVFKPFEKPVMISMGNENVLEIK
GNDIDPEAVKGEVLKVGNKSCENIHLHSEAVLCTVPNDLLKLNSELNIEW
KQAISSTVLGKVIVQPDQNFTGLIAGVVSISTALLLLLGFFLWLKKRKQI
KDLGSELVRYDARVHTPHLDRLVSARSVSPTTEMVSNESVDYRATFPEDQ
FPNSSQNGSCRQVQYPLTDMSPILTSGDSDISSPLLQNTVHIDLSALNPE
LVQAVQHVVIGPSSLIVHFNEVIGRGHFGCVYHGTLLDNDGKKIHCAVKS
LNRITDIGEVSQFLTEGIIMKDFSHPNVLSLLGICLRSEGSPLVVLPYMK
HGDLRNFIRNETHNPTVKDLIGFGLQVAKGMKYLASKKFVHRDLAARNCM
LDEKFTVKVADFGLARDMYDKEYYSVHNKTGAKLPVKWMALESLQTQKFT
TKSDVWSFGVLLWELMTRGAPPYPDVNTFDITVYLLQGRRLLQPEYCPDP
LYEVMLKCWHPKAEMRPSFSELVSRISAIFSTFIGEHYVHVNATYVNVKC
VAPYPSLLSSEDNADDEVDTRPASFWETS

Of which:

(SEQ ID NO: 148)
MKAPAVLAPGILVLLFTLVQRSNG: signal peptide
(SEQ ID NO: 149)
ECKEALAKSEMNVNMKYQLPNFTAETPIQNVILHEHHIFLGATNYIYVLN
EEDLQKVAEYKTGPVLEHPDCFPCQDCSSKANLSGGVWKDNINMALVVDT
YYDDQLISCGSVNRGTCQRHVFPHNHTADIQSEVHCIFSPQIEEPSQCPD
CVVSALGAKVLSSVKDRFINFFVGNTINSSYFPDHPLHSISVRRLKETKD
GFMFLTDQSYIDVLPEFRDSYPIKYVHAFESNNFIYFLTVQRETLDAQTF
HTRIIRFCSINSGLHSYMEMPLECILTEKRKKRSTKKEVFNILQAAYVSK
PGAQLARQIGASLNDDILFGVFAQSKPDSAEPMDRSAMCAFPIKYVNDFF
NKIVNKNNVRCLQHFYGPNHEHCFNRTLLRNSSGCEARRDEYRTEFTTAL
QRVDLFMGQFSEVLLTSISTFIKGDLTIANLGTSEGRFMQVVVSRSGPST
PHVNFLLDSHPVSPEVIVEHTLNQNGYTLVITGKKITKIPLNGLGCRHFQ
SCSQCLSAPPFVQCGWCHDKCVRSEECLSGTWTQQICLPAIYKVFPNSAP
LEGGTRLTICGWDFGFRRNNKFDLKKTRVLLGNESCTLTLSESTMNTLKC
TVGPAMNKHFNMSIIISNGHGTTQYSTFSYVDPVITSISPKYGPMAGGTL
LTLTGNYLNSGNSRHISIGGKTCTLKSVSNSILECYTPAQTISTEFAVKL
KIDLANRETSIFSYREDPIVIEIHPTKSFISGGSTITGVGKNLNSVSVPR
MVINVHEAGRNFTVACQHRSNSEIICCTTPSLQQLNLQLPLKTKAFFMLD
GILSKYFDLIYVHNPVFKPFEKPVMISMGNENVLEIKGNDIDPEAVKGEV
LKVGNKSCENIHLHSEAVLCTVPNDLLKLNSELNIEWKQAISSTVLGKVI
VQPDQNFT: ECD of human cMET
(SEQ ID NO: 150)
GLIAGVVSISTALLLLLGFFWL: transmembrane region
(SEQ ID NO: 151)
KKRKQIKDLGSELVRYDARVHTPHLDRLVSARSVSPTTEMVSNESVDYRA
TFPEDQFPNSSQNGSCRQVQYPLTDMSPILTSGDSDISSPLLQNTVHIDL
SALNPELVQAVQHVVIGPSSLIVHFNEVIGPGHFGCVYHGTLLDNDGKKI
HCAVKSLNRITDIGEVSQFLTEGIIMKDFSHPNVLSLLGICLRSEGSPLV
VLPYMKHGDLRNFIRNETHNPTVKDLIGFGLQVAKGMKYASKKFVHRDLA
ARNCMLDEKFTVKVADFGLARDMYDKEYYSVHNKTGAKLPVKWMALESLQ
TQKFTTKSDVWSFGVLLWELMTRGAPPYPDVNTFDITVYLLQGRRLLQPE
YCPDPLYEVMLKCWHPKAEMRPSFSELVSRISAIFSTFIGEHYVHVNATY
VNVKCVAPYPSLLSSEDNADDEVEDTRPASFWETS: intracellular
region

### Reference antibodies

Anti-cMET Antibodies are known in the art (Table 1). Monospecific bivalent cMET antibodies were constructed according to published information and expressed in 293F Freestyle cell. Table 1 shows the related disclosed information. Monospecific bivalent antibodies directed against cMET were constructed according to published information and expressed in 293F Freestyle cells. For HGF ligand blocking assays VH- and VL-encoding gene segments of patent-derived anti-cMET antibodies were re-cloned in a phage display vector for display on filamentous bacteriophage.

Reference antibody cetuximab (Erbitux) was used as reference antibody for the EGFR Fab panel.

2994 Fab protein was generated from purified PG2994 IgG by papain digestion. Therefore PG2994 was incubated with papain coupled on beads (Pierce #44985), and allowed to digest for 5.5 hour at 37° C. under rotation. Fab fragments were purified from the digestion mixture by filtration over MabSelectSure LX. Flow through fractions containing Fab protein, concentrated to 3 ml using vivaspin20 10 kDa and further purified by gel filtration using a superdex75 16/600 column in PBS.

### Example 2

### Generation of Bivalent Monoclonal Antibodies and Antibody Characterization

VH genes of unique antibodies, as judged by VH gene sequence and some sequence variants thereof, were cloned in the backbone IgG1 vector. Suspension adapted 293F Freestyle cells were cultivated in T125 flasks at a shaker plateau until a density of 3.0×10⁶ cells/ml. Cells were seeded at a density of 0.3-0.5×10⁶ viable cells/ml in each well of a 24-deep well plate. The cells were transiently transfected with the individual sterile DNA: PEI mixture and further cultivated. Seven days after transfection, supernatant was harvested and filtrated through 0.22 μM (Sartorius) and purified on protein A beads using batch purification followed by a buffer exchange to PBS.

### Inhibition of EGF Mediated Apoptosis

High (10 nM) concentrations of EGF induce (apoptotic) cell death in A431 cells [Gulli et al., 1996)]. This effect can be dose-dependently reverted by the addition of ligand-blocking anti-EGFR antibodies, such as cetuximab.

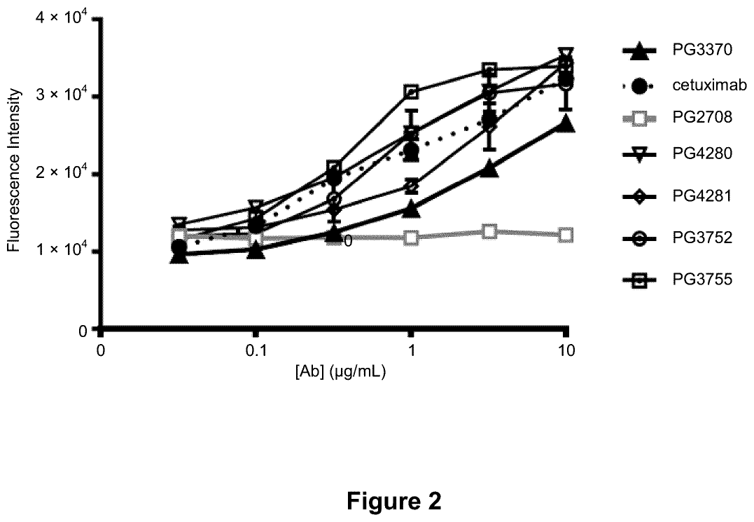

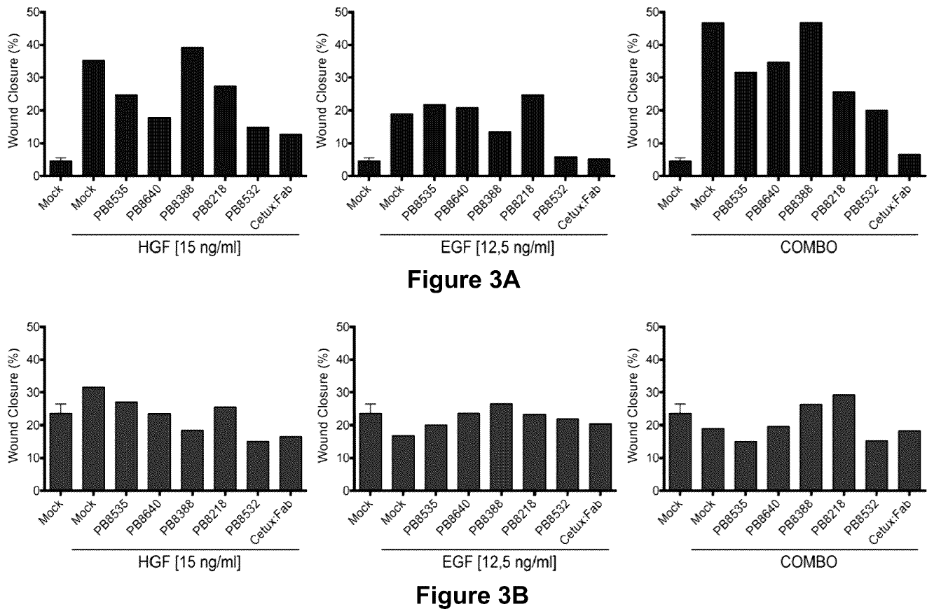

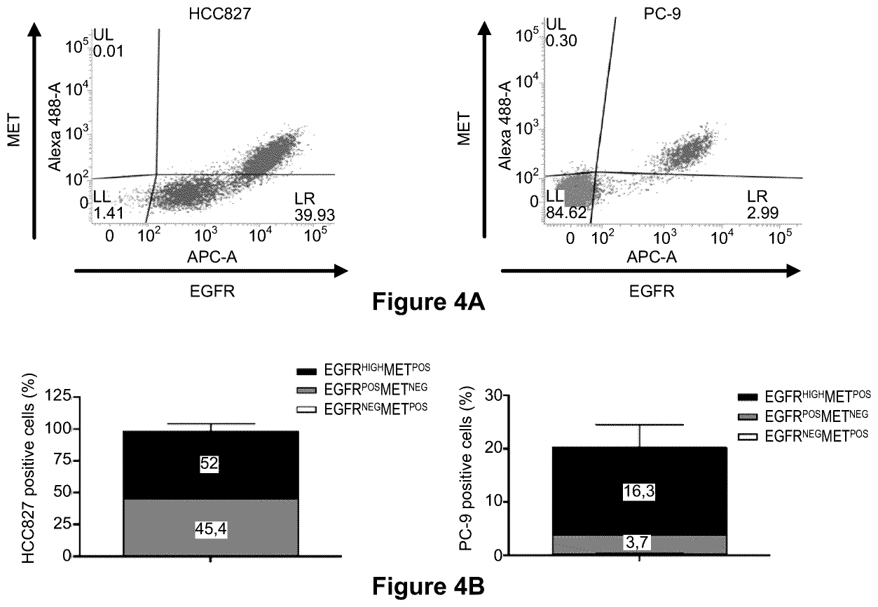

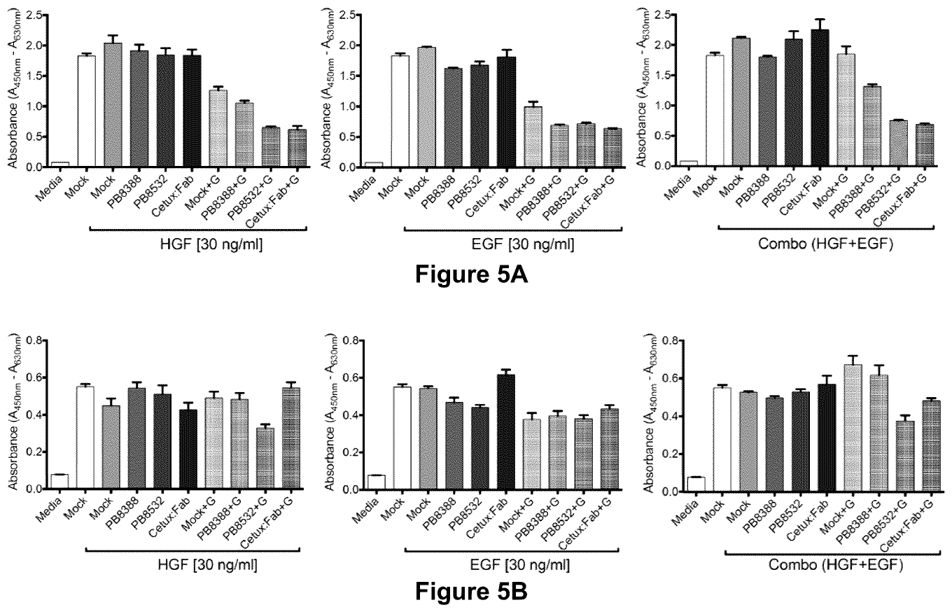

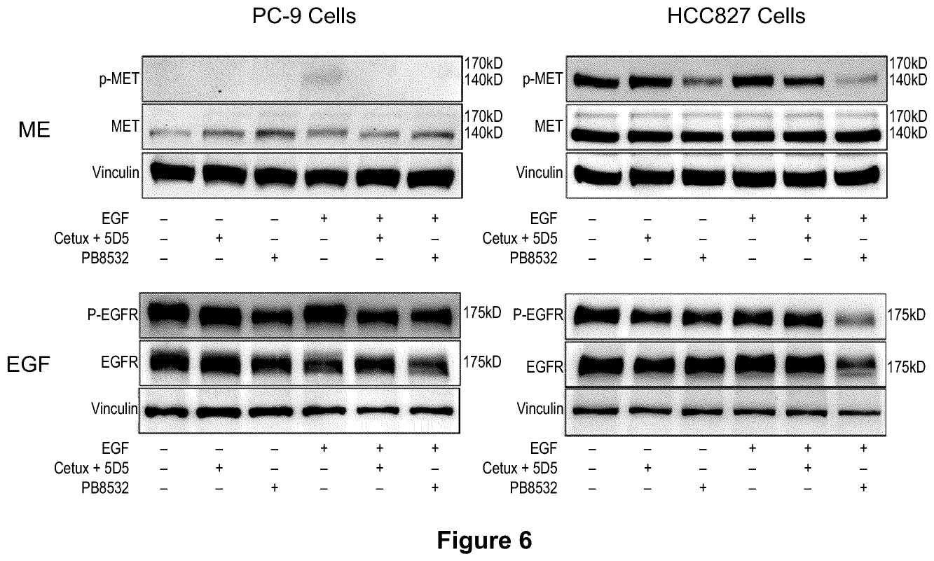

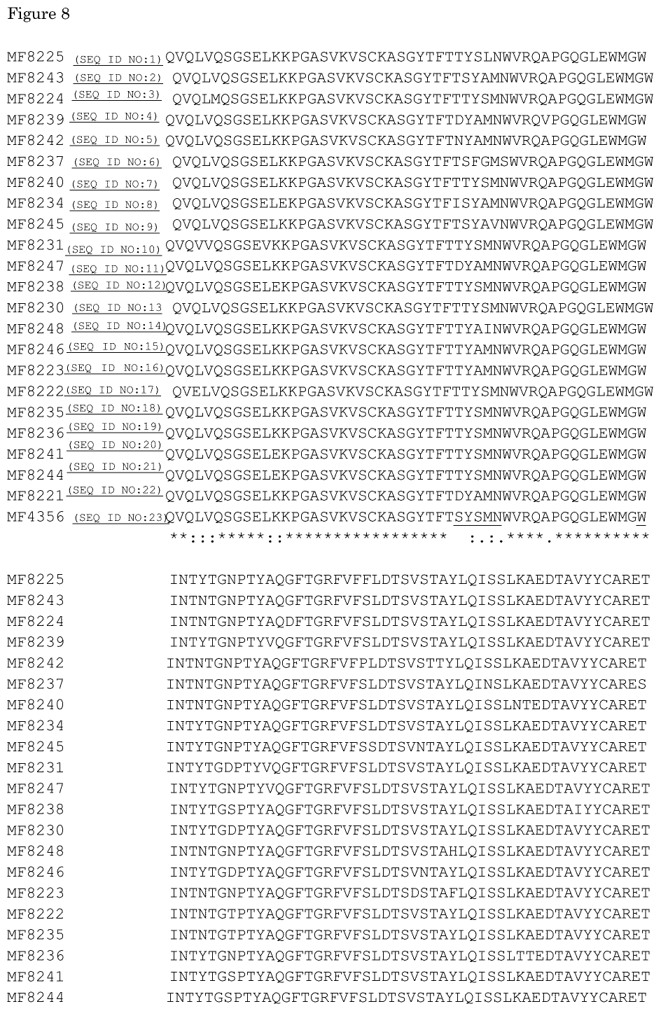

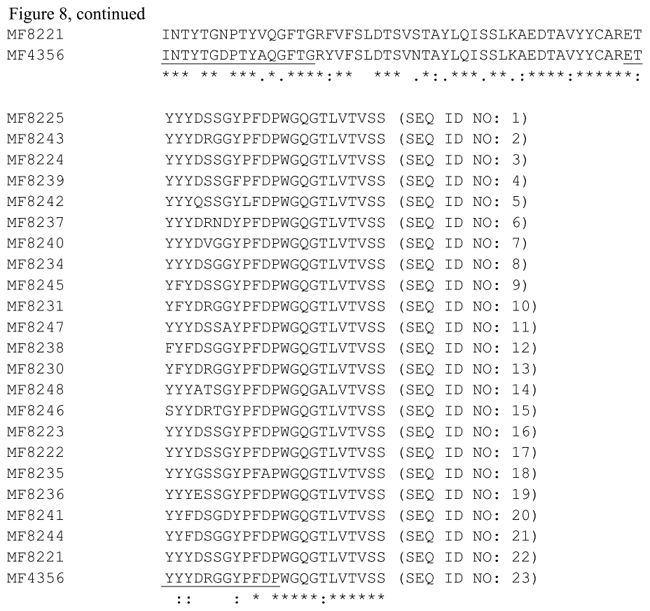

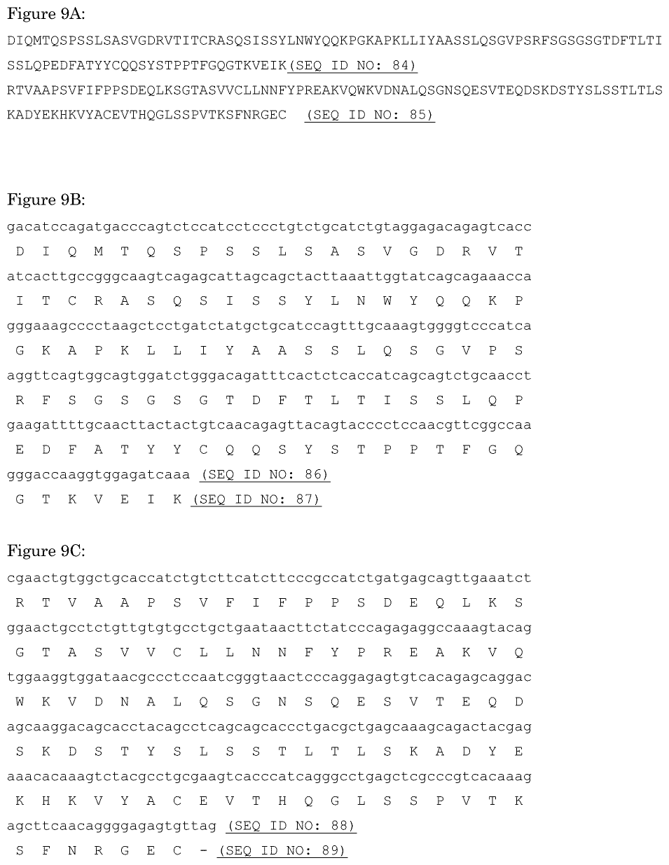

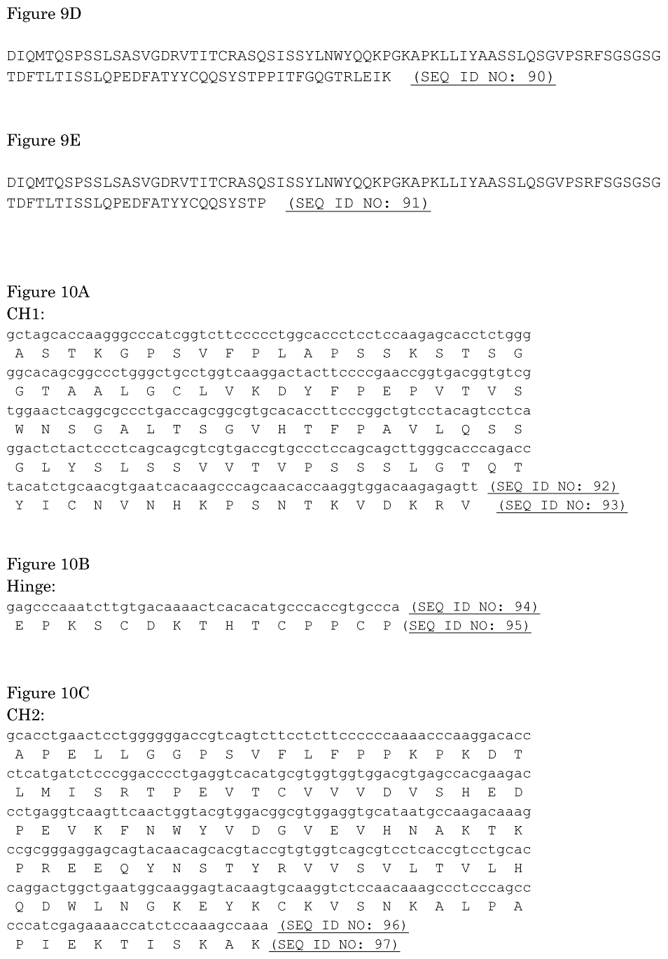

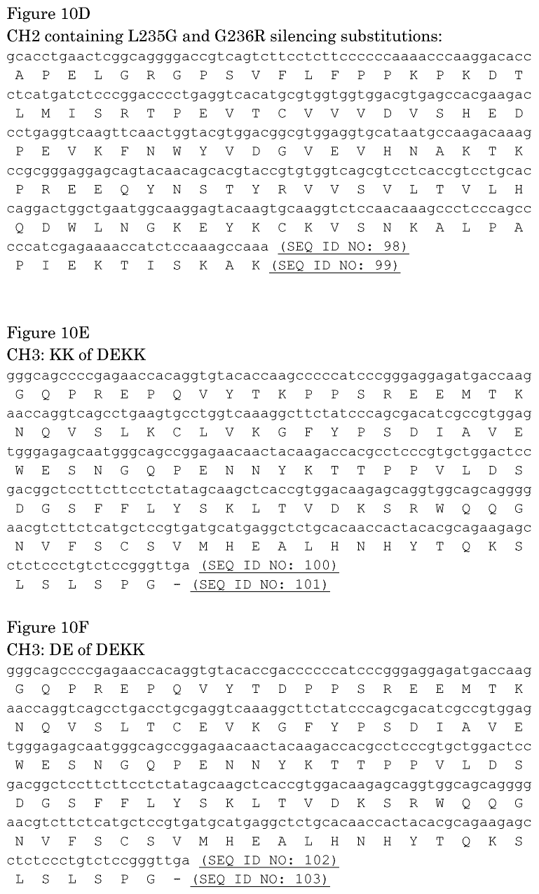

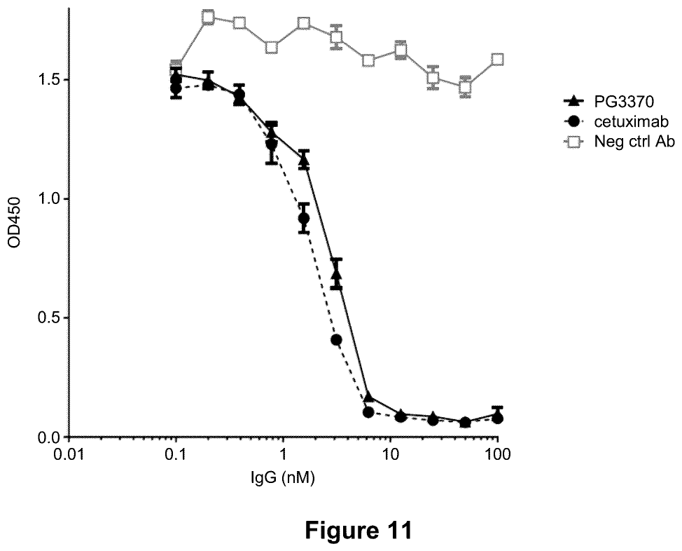

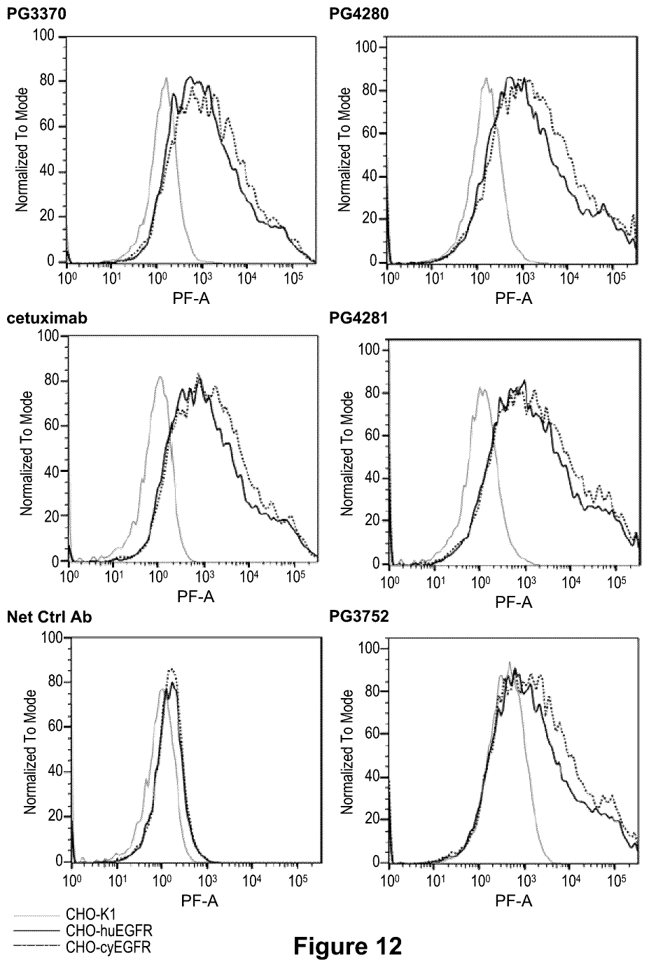

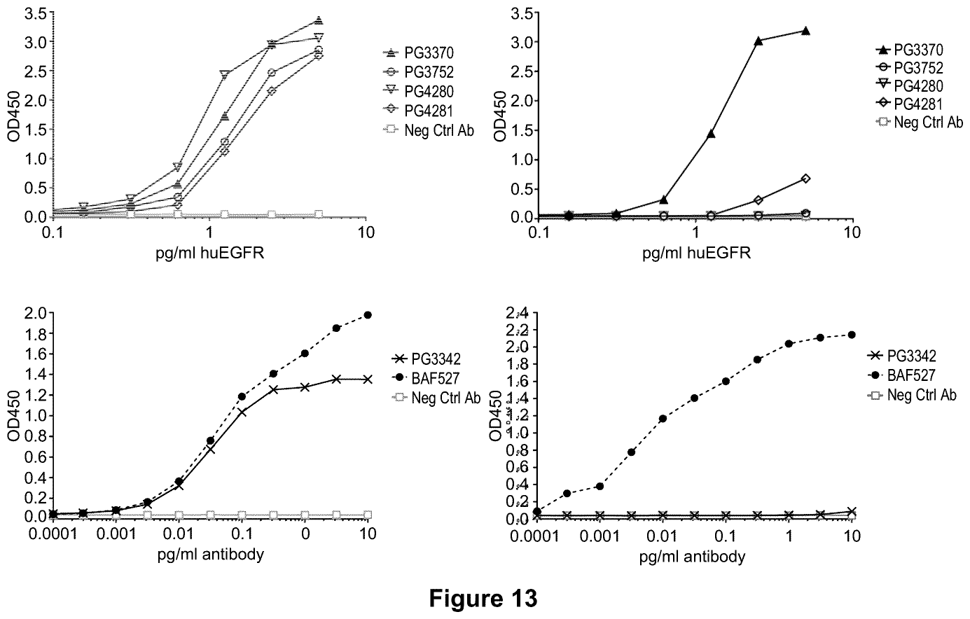

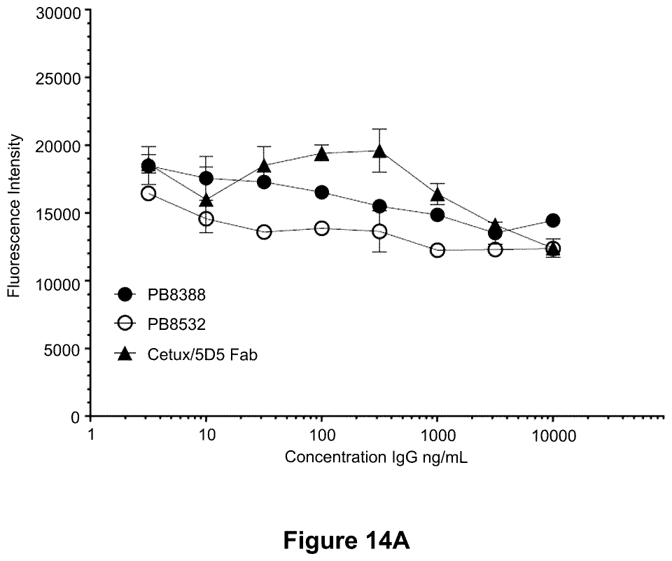

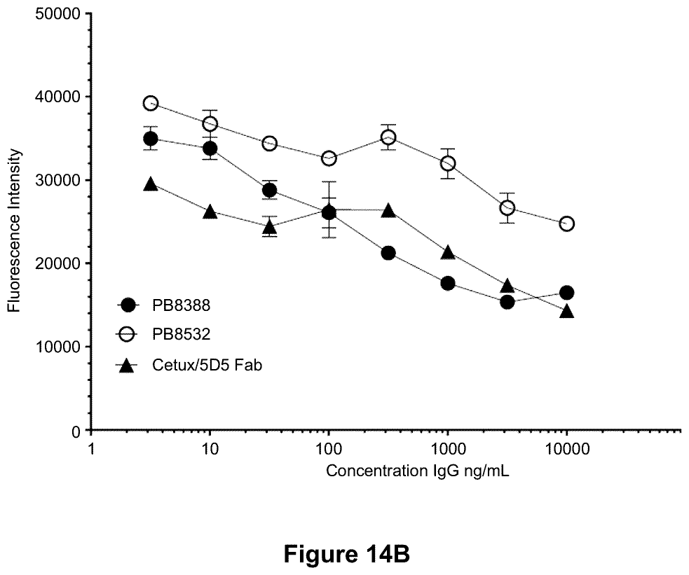

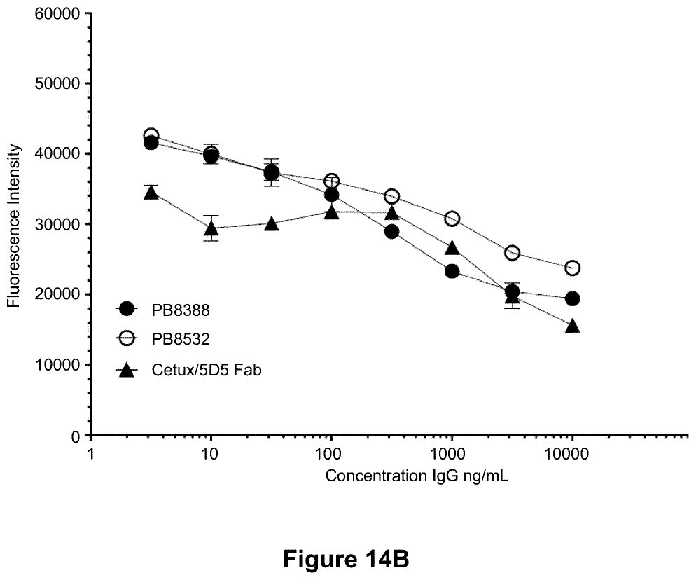

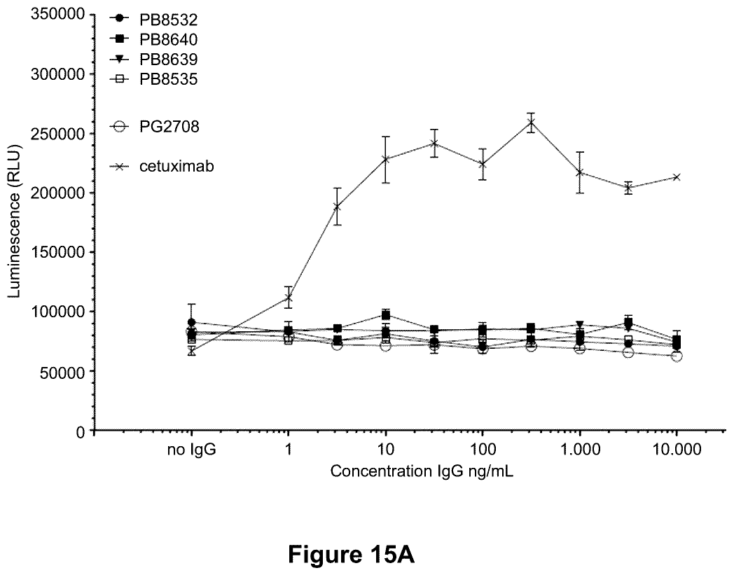

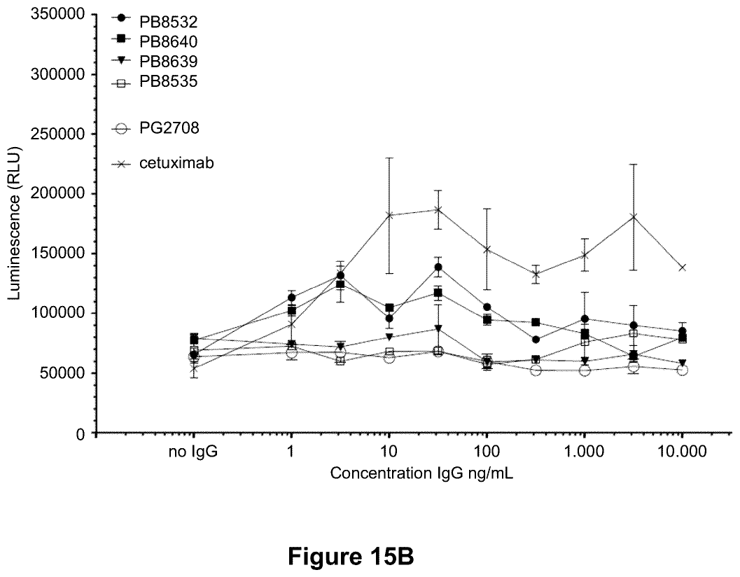

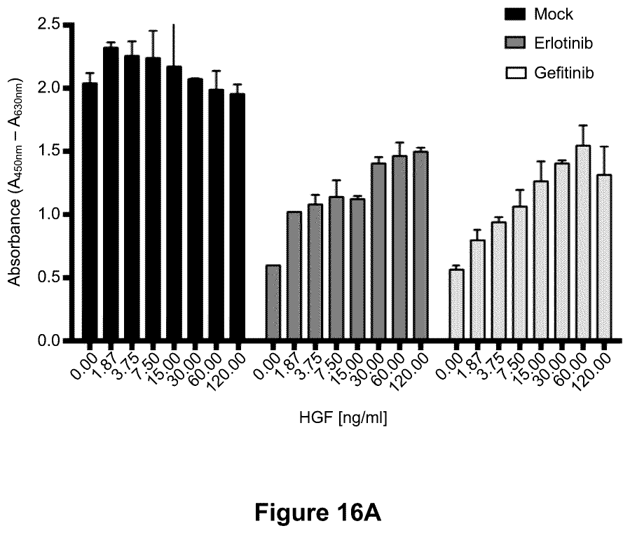

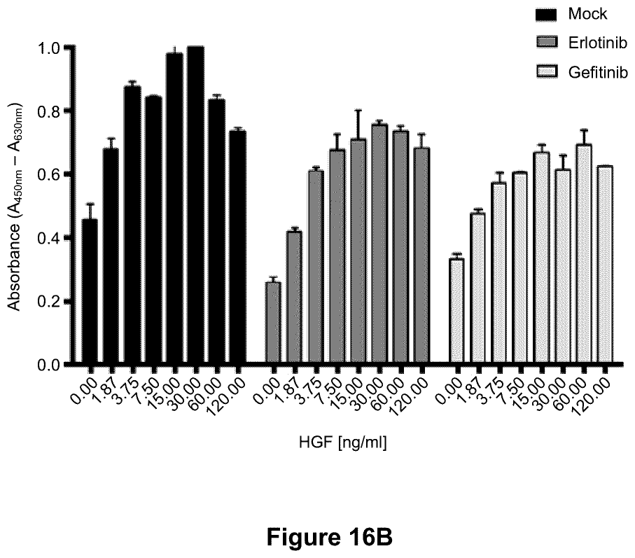

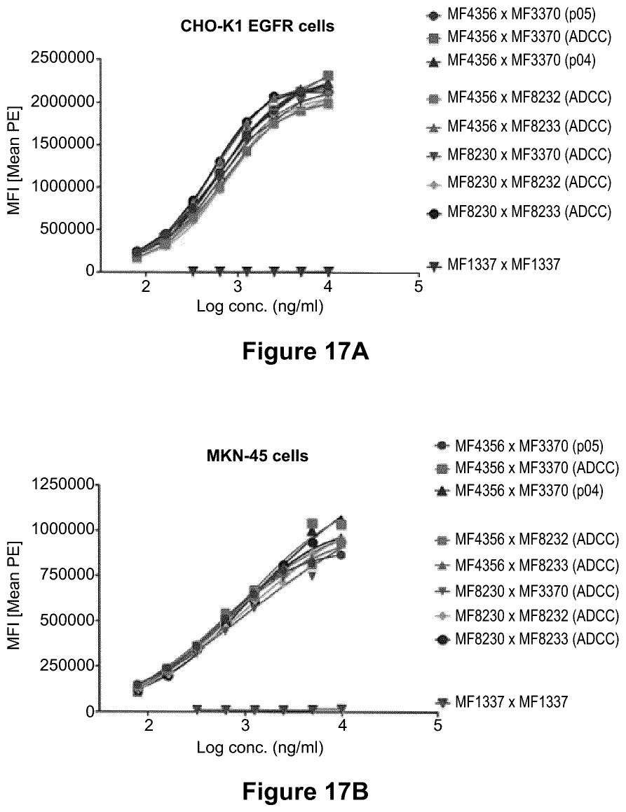

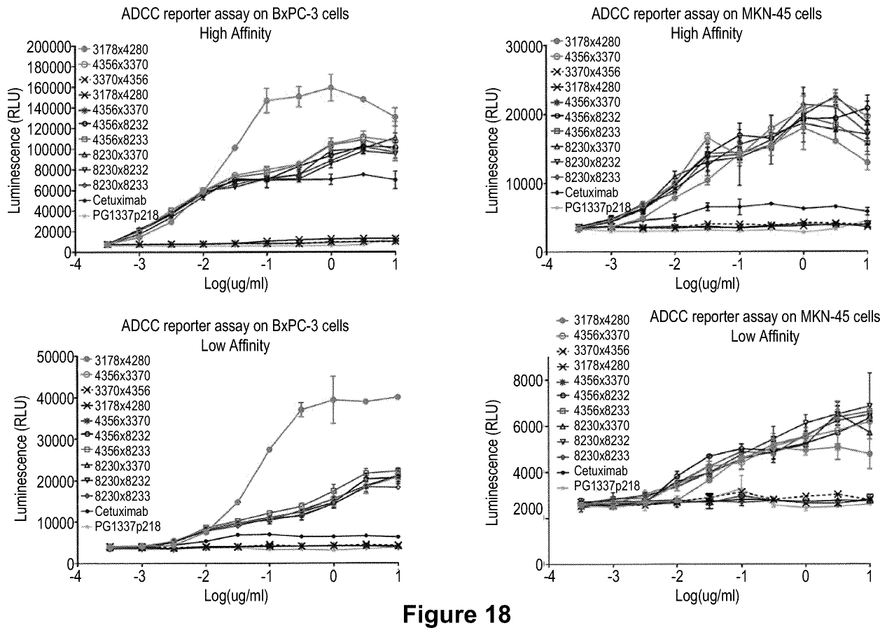

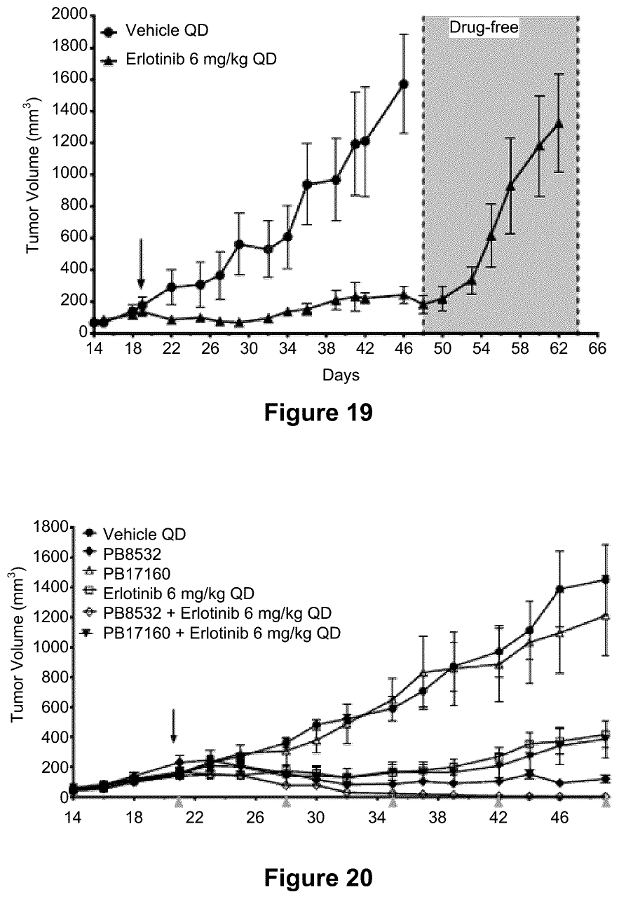

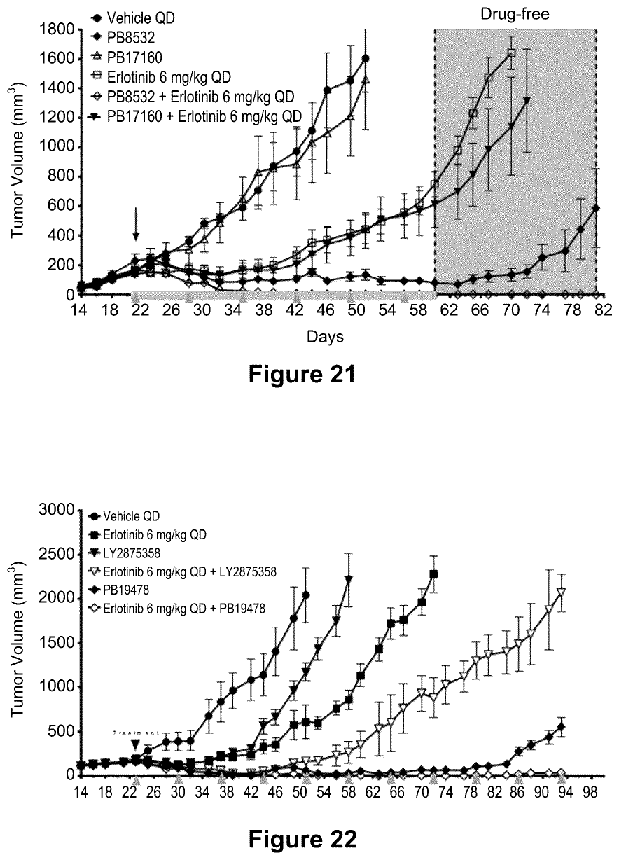

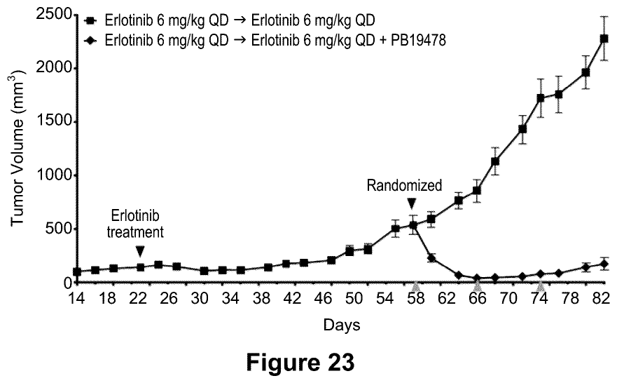

To test the bivalent anti-EGFR IgG for their potency to inhibit EGF-induced cell death of A431 cells, antibodies were incubated in a serial titration—from 10 μg/ml onwards—in the presence of 10 nM EGF. Each assay plate contained a serial dilution of negative (Ctrl Ab; PG2708) and positive control antibody (cetuximab) that served as reference controls. On the third day, Alamar blue (Invitrogen, # DAL1100) was added (20 μl per well) and the fluorescence was measured after 6 hours of incubation (at 37° C.) with Alamar blue using 560 nm excitation and 590 nm readout on a Biotek Synergy 2 Multi-mode microplate reader. FIG. 2 shows the activity of the cLC EGFR antibodies compared to that of cetuximab and the control antibody. Antibodies PG4280, 3755 and 3752 were more potent in comparison to cetuximab whereas antibodies PG4281 and PG3370 showed less efficacy.

### EGF blocking ELISA

EGFR specific phages were tested for binding to recombinant EGFR in the absence and presence of a molar excess of ligand (EGF). Therefore 5 μg/ml of goat anti-human IgG was coated overnight to MAXISORP™ ELISA plates at 4° C. Wells of the ELISA plates were blocked with PBS (pH 7.2) containing 2% ELK for 1 h at RT while shaking (700rpm). Next, 5 μg/ml recombinant human EGFR-Fc was allowed to incubate for 1 H at RT. Meanwhile, IgG was mixed in a serial titration with human biotinylated EGF for 1 H at RT. After washing away unbound human EGFR-Fc, the antibody/EGF mixture was added and allowed to bind for 1 H at RT. Bound EGF was detected by HRP-streptavidine for 1 H at RT. As a control the procedure was performed simultaneously with an antibody specific for the coated antigens (not shown) and a negative control phage (Neg Ctrl Ab). Bound secondary antibody was visualized by TMB/H₂O₂ staining and staining was quantified by means of OD₄₅₀ₙₘ measurement. FIG. 11 depicts that the PG3370 antibody, which is less potent in the inhibition of EGF mediated apoptosis, shows similar EGF blocking activity in comparison cetuximab.

### Cynomolgus EGFR and Mouse EGFR Cross Reactivity Test

To test whether anti-EGFR IgGs were reactive with cynomolgus EGFR, the constructs encoding full-length human EGFR, as well as the expression construct encoding the ECD of cynomolgus fused to intracellular human EGFR, were both transfected in (antigen negative) CHO cells and cells were then stained with the anti-EGFR antibodies at 5 μg/ml and finally analysed by FACS. As a positive control for the staining, the clinically used antibody cetuximab was used, as this antibody is known to cross-react with cynomolgus EGFR. PG3370, PG3752, PG4280 and PG4281 were shown to be reactive with cynomolgus EGFR, as the staining of cells expressing human EGFR was virtually indistinguishable from that of cells expressing the chimeric receptor (FIG. 12).

To test anti-EGFR IgG for their cross-reactivity with murine EGFR, an ELISA was performed. A serial titration of recombinant mouse EGFR ECD-Fc, starting at 5 μg/ml and diluted until 0.038 μg/ml was coated overnight to MAXISORP™ ELISA plates at 4° C. Binding of the anti-EGFR IgG to this antigen was tested at a fixed concentration of 5 μg/ml and allowed to bind for 1 H at RT. As a positive control for the immuno-reactivity of the antibodies, the same ELISA setup was performed using the human EGFR ECD-Fc fusion protein as antigen (R&D systems). Next, Goat anti-mouse IgG HRP conjugate, BD Biosciences) and was allowed to bind for 2 hours at RT. Bound IgG was detected by means of OD450 nm measurement. Antibody PG3370 was shown to recognize murine EGFR, as well as human EGFR with similar affinity (FIG. 13-Upper panel). Cetuximab does not recognize mouse EGFR (125084 Erbitux Pharmacology Review Part 2—FDA). PG3370 and cetuximab thus do not recognize the same epitope on human EGFR.

### Mouse cMET Cross Reactivity Test

To test PG3342 for its cross-reactivity to murine cMET, an ELISA was performed. A fixed concentration of mouse HGF R/c-MET Fc (R&D systems) HGF R/c-MET Fc was diluted to 2.5 μg/ml in PBS and coated overnight to MAXISORP™ ELISA plates at 4° C. Binding of the anti-cMET IgG to this antigen was tested in a semi-log titration starting at 10 μg/ml. Antibodies were allowed to bind for 1 H at RT. As a positive control for the immuno-reactivity of the antibodies, the same ELISA setup was performed using the human HGF R/c-MET Fc fusion protein as antigen (R&D systems). Next, Goat anti-mouse IgG HRP conjugate (BD Biosciences) was added and allowed to bind for 2 hours at RT. Bound IgG was detected by means of OD450 nm measurement. BAF527 an antigen affinity-purified Polyclonal Goat IgG directed against mouse cMET coupled to biotin was included as a positive control antibody. No cross reactivity to murine cMET was observed with the PG3342 antibody (FIG. 13 lower panel).

Cross block assay cMET antibodies

cMET specific phages were tested for competition with cMET reference antibodies in ELISA. Therefore 2.5 μg/ml of cMET-Fc fusion protein was coated overnight to MAXISORP™ ELISA plates at 4° C. Wells of the ELISA plates were blocked with PBS (pH 7.2) containing 2% ELK for 1 H at RT while shaking (700 rpm). Next reference or negative control IgG was added at a concentration of 5 μg/ml and allowed to bind for 15 min at RT at 700 rpm. Next, 5 μl of PEG precipitated phage was added and allowed to bind for 1 H at RT at 700 rpm. Bound phages were detected with HRP labelled anti-M13 antibody for 1 H at RT at 700 rpm. As a control the procedure was performed simultaneously with an antibody specific for the coated antigens and a negative control phage. Bound secondary antibody was visualized by TMB/H202 staining and staining was quantified by means of OD₄₅₀ₙₘ measurement. Table 2 demonstrates that MF4040 and MF4356 show competition with the 5D5 reference antibody. MF4297 competes with 13.3.2 and C8H241 to a lesser extent. The positive control phages all show complete competition with the corresponding IgG, whereas the no antibody control, does not influence the competition assay.

### Generation of Bispecific Antibodies

Bispecific antibodies were generated by transient co-transfection of two plasmids encoding IgG with different VH domains, using a proprietary CH3 engineering technology to ensure efficient heterodimerisation and formation of bispecific antibodies. The common light chain is also co-transfected in the same cell, either on the same plasmid or on another plasmid. In our co-pending applications (e.g. WO2013/157954 and WO2013/157953; incorporated herein by reference) we have disclosed methods and means for producing bispecific antibodies from a single cell, whereby means are provided that favor the formation of bispecific antibodies over the formation of monospecific antibodies. These methods can also be favorably employed in the present invention. Specifically, preferred mutations to produce essentially only bispecific full length IgG molecules are amino acid substitutions at positions 351 and 366, e.g. L351K and T366K (numbering according to EU numbering) in the first CH3 domain (the ‘KK-variant’ heavy chain) and amino acid substitutions at positions 351 and 368, e.g. L351D and 10 L368E in the second CH3 domain (the ‘DE-variant’ heavy chain), or vice versa. It was previously demonstrated in our co-pending applications that the negatively charged DE-variant heavy chain and positively charged KK- variant heavy chain preferentially pair to form heterodimers (so-called ‘DEKK’ bispecific molecules). Homodimerization of DE-variant heavy chains (DE-DE homodimers) or KK-variant heavy chains (KK-KK homodimers) are disfavored due to strong repulsion between the charged residues in the CH3-CH3 interface between identical heavy chains.

cMET and EGFR Fab arms were cloned in the appropriate KK and DE vectors (Table 3). After production, bispecific IgG were purified by protein-A batch purification and the buffer was exchanged to PBS. Successful productions resulted in an IgG1 full length antibody, with a minimal concentration of 0.1 mg/ml, which were assigned a unique code (PBnnnnn; where nnnnn represents a randomly generated number) to identify the specific combination of 2 different target binding Fab fragments. Successfully produced bispecific IgG were tested for binding to their respective targets in ELISA.

### Example 3

### Screening of c-MET x EGFR Bispecific Antibodies in an EGF/HGF and HGF and EGF Proliferation Assay

The potency of a panel of cMET x EGFR bispecific antibodies was tested in N87 cells using an HGF/EGF, HGF and EGF assays. The N87 cell line, official name NCI-N87, is a gastric carcinoma cell line derived from a metastatic site and has high EGFR expression levels and intermediate cMET expression levels (Zhang et al, 2010). Antibodies were tested in an 8 steps semi-log titration ranging from 10 μg/ml to 3.16 ng/ml. Each antibody was tested in duplicate. The anti-RSV-G antibody PG2708 was used as negative control. The reference antibody 2994 Fab was used as positive control for the HGF assay and the reference antibody cetuximab was used as positive control for the EGF assay.

An equimolar 1:1 cetuximab/5D5 Fab was used as positive control for the EGF, HGF and EGF/HGF assays.

Wells with either one, or a combination of ligand, as well as medium control were included to determine the assay window. Antibodies were diluted in chemically defined starvation medium (CDS: RPMI1640 medium, containing 80 U penicillin and 80 μg of streptomycin per ml, 0.05% (w/v) BSA and 10 μg/ml holo-transferrin) and 50 μl of diluted antibody was added to the wells of a 96 wells black well clear bottom plate (Costar). Ligand was added (50 μl per well of a stock solution containing 400 ng/ml HGF and 4 ng/ml of EGF, and a EGF/HGF concentration of 4 ng/ml EGF/400 ng/ml HGF diluted in CDS: R&D systems, cat. nr. 396-HB and 236-EG). N87 cells were trypsinised, harvested and counted and 8000 cells in 100 μl of CDS were added to each well of the plate. To avoid edge effects, plates were left for an hour at RT before being put in a container inside a 37° C. cell culture incubator for three days. On the fourth day, Alamar blue

(Invitrogen, # DAL1100) was added (20 μl per well) and the fluorescence was measured after 6 hours of incubation (at 37° C.) with Alamar blue using 560 nm excitation and 590 nm readout on a Biotek Synergy 2 Multi-mode microplate reader. Fluorescence values were normalised to uninhibited growth (no antibody, but both ligands added). An example of an HGF, EGF and EGF/HGF proliferation assay is shown in FIG. 14 (FIG. 14A, B and C respectively).

Table 4 lists the results of the various experiments. In the N87 HGF/EGF assay, fourteen different cMETxEGFR bispecifics with potency comparable to the reference monospecific antibodies (equimolar mix of cetuximab and 5D5 Fab) were identified: PB7679, PB7686, PB8218, PB8244, PB8292, PB8316, PB8340, PB8364, PB8388, PB8511, PB8535, PB8583, PB8607 and PB8640.

In the N87 EGF assay, eleven different cMETxEGFR bispecifics with potency comparable to monospecific cetuximab were identified: PB7679, PB8244, PB8292, PB8340, PB8364, PB8388, PB8511, PB8535, PB8583, PB8607 and PB8640. They all contain the EGFR Fab arm MF3755. In the HGF N87 assay nine bispecifics were identified that showed a higher potency compared to the monospecific 5D5 Fab reference antibody: PB8218, PB8388, PB8511, PB8532, PB8535, PB8545, PB8583, PB8639 and PB8640. They contain six different cMET Fab arms MF4040, MF4297, MF4301, MF4356, MF4491 and MF4506.

### ADCC Activity

The ADCC activity of the 24 cMetxEGFR bispecifics was tested to the tumor cell lines N87 (EGFR-high, cMET-low) and MKN-45 (EGFR-low, cMET-amplified). The ADCC assay was performed using the Promega ADCC Bioassay kit in 384-well plate format. Antibodies were tested in duplicate at 9 different concentrations in semi-log serial dilutions ranging from 10 μg/ml to 1 ng/ml.

The reference cetuximab antibody was included as a positive control for the assay and PG2708 was used as negative control antibody. Antibodies or assay medium control (no IgG) were incubated for 6 hours of induction at 37° C. with ADCC effector cells, and target cells (N87 or MKN-45). Luciferase activity was quantified using Bio-Glo luciferase reagent.

An example of the ADCC assay is shown in FIG. 15. None of the cMETxEGFR bispecifics showed a significant ADCC activity in both cell lines. The positive control reference cetuximab antibody showed a dose-dependent ADCC activity to both cell lines.

Five bispecifics composed of EGFR and cMet arms which did show high efficacy in the N87 HGF/EGF assay and showed high sequence diversity (Table 5) were selected for further analysis. Two from the five bispecifics contain MF4356, which competes with 5D5 for binding to cMET (Table 2). Table 5 summarizes the characteristics of the selected candidates.

### Wound Healing Cell Migration Assay

Two NSCLC cell lines were tested in the wound healing assay; EBC-1 and H358. These cell lines were chosen since they express high levels of EGFR and c-Met (Zhang et al, 2010; Fong et al., 2013). The assay was performed using the CytoSelect™ 24-well plate wound healing assay (Cell Biolabs, CBA-120) according to the manufacturer's instructions. Briefly, 2.5-4×10⁵ cancer cells were seeded in each well and incubated overnight at 37° C. to form a monolayer. Well inserts were then removed to create a wound field of 0.9-mm After washing with PBS to remove dead cells and debris in the wound area, cells were incubated for 15 minutes at 37° C. with complete media (0.5% FBS) containing bispecifics (100 nM) or cetuximab:Fab2994 control antibody mixture (100 nM, 1:1 molar ratio). Each well was then supplemented with growth factors: HGF (15 ng/ml), EGF (12.5 ng/ml) or a combination of both (15 and 12.5 ng/ml). Time-lapse monitoring of the wound closure was performed for 14 h at 37° C. with a confocal microscope (Zeiss LSM780). The extent (%) of wound closure is shown relative to untreated controls.

H358 cells showed in an increase in migration (percentage wound closure) upon exposure to either HGF or EGF alone, which was most effective by a combination of HGF and EGF (FIG. 3). This increased migration was abrogated by the addition of the majority of the bispecifics and was most pronounced with PB8532. This inhibition was comparable to the cetuximab and 5D5 Fab combination except for the inhibition migration in the presence of EGF/HGF.

EBC-1 cells showed a slight increase in migration by addition of HGF; and no increase in migration in the presence of EGF or the combination of EGF/HGF. However wound closure could be inhibited in all assay conditions by the bispecifics in particular by PB8532. PB8532 was as effective or more effective (EGF/HGF) than the combination of cetuximab/5D5 Fab.

### Analysis of EGFR and cMET Expression on PC-9 and HCC827 Cells by Flow Cytometry

Acquired resistance to erlotinib can result from aberrant activation of HGF mediated c-MET activation. The NSCLC cell lines PC-9 and HCC827 were selected to investigate the ability of the cMET xEGFR bispecific antibodies to inhibit ligand mediated proliferation in a tyrosine kinase inhibitor (TKI) resistant setting. Both cell lines do not harbor EGFR mutations and are resistant in the presence of HGF to both erlotinib and gefitinib or a combination of gefinitib and erlotinib (PC-9 only). In PC-9 cells, it has been reported that the HGF-induced erlotinib resistance can be abrogated by a cMET inhibitor (Nakade et al., 2014) and the gefitinib resistance can be abrogated by an anti-HGF antibody (Yano, 2008). PC-9 and HCC827 were characterized for the expression of cMET and EGFR by FACS analysis, using fluorescently labeled antibodies. Cells were harvested with PBS 2 mM EDTA. Single-cell suspensions (10e6 cells in 50 μl) were incubated with fluorescently labelled antibodies on ice for 20 min in staining buffer (PBS 2% FBS 2 mM EDTA). The following antibodies were used alone or in combination: Met Alexa Fluor 488 conjugate (Clone D1C2, Cell Signaling, 1:50 dilution); EGF Receptor Alexa Fluor 647 conjugate (Clone D38B1, Cell Signaling, 1:50 dilution). After incubation, cells were washed with staining buffer and FACS analysis was performed on a BD FACSVerse flow cytometer.

All HCC827 cells show EGFR expression and can be subdivided into a EGFR^{high}, cMET^{pos} population and a EGFR^{pos}, cMET^{neg} population (FIG. 4). PC-9 cells contain a small population of EGFR^{high} and cMET^{pos} cells and a minimal population of EGFR^{pos} and cMET^{neg} cells.

### PC-9 and HCC827 Proliferation Assay

Initial experiments were performed to determine the concentration of HGF establishing erlotinib and gefitinib resistance in PC-9 and HCC827 cells. Upon overnight starvation in media containing 0.5% FBS, cells were incubated with increasing concentrations of HGF, ranging from 0 to 120 ng/mL supplemented with 300 nM erlotinib or gefitinib, in 10% FBS. After 72 hours of incubation, cell proliferation was assessed using the WST-1 reagent according to the manufacture's instructions. The absorbance was measured with a microplate reader at test and reference wavelengths of 450 and 630 nm, respectively. In both PC-9 (FIG. 16A) and HCC287 (FIG. 16B) the addition of HGF induced resistance to TKIs in a dose-dependent manner.

PB8532 and PB8388 were tested for their efficacy in a TKI resistance setting. 4×10³ cancer cells were seeded in 96-well plates in 100 μL complete RPMI 1640 (10% FBS). Upon overnight starvation in media containing 0.5% FBS, cells were pre-incubated for 15 minutes at 37° C. with Biclonics® (100 nM) or cetuximab:Fab2994 control monospecific antibody mixture (100 nM, 1:1 molar ratio). Each well was then supplemented with complete media (10% FBS) containing gefitinib or erlotinib (300 nM) with or w/o HGF (30 ng/ml), EGF (30 ng/ml) or a combination of both (at 30 ng/ml). After 72 hours of incubation, cell proliferation was assessed using the WST-1 method.

FIG. 5 shows that PB8532 can inhibit HGF mediated and EGF mediated gefitinib resistance in PC-9 cells. In HCC827 cells PB8532 inhibited HGF mediated TKI resistance, and was more potent than the combination of administering dual monospecific cetuximab 5D5 Fab. Comparable results were obtained with the TKI erlotinib.

### Example 4 PB8532 Inhibition of EGF and cMET Phosphorylation

Following overnight starvation in media without FBS, cells were incubated for 15 minutes at 37° C. with media (0.5% FBS) containing PB8532 (100 nM) or cetuximab:Fab2994 control monospecific antibody mixture (100 nM, 1:1 molar ratio). The cells were then stimulated with growth factors: HGF (30 ng/ml) or EGF (50 ng/ml) for 15 minutes at 37° C. After stimulation, cells were washed with PBS in presence of 1 mM orthovanadate (Sigma-Aldrich). Protein extraction was performed using RIPA lysis buffer (50 mM Tris HCl pH 8, 150 mM NaCl, 1% Triton X-100, 0.5% sodium deoxycholate, 0.1% SDS) supplemented with Complete Protease Inhibitor Cocktail (Roche), PhosSTOP phosphatase inhibitor (Roche) and orthovanadate (1 mM, Sigma-Aldrich). Lysates were incubated on ice for 30 minutes before centrifuging 15 minutes at 4° C. to remove cellular debris. After centrifugation, the supernatant was collected and protein concentrations were determined using bicinchoninic acid (BCA) reagent (Pierce) according to the manufacturer's instructions. Protein samples were denaturated by adding loading buffer 6× (β-mercaptoethanol 0.6 M; SDS 8%; Tris-HCl 0.25 M pH 6.8; glycerol 40%; Bromophenol Blue 0.2%) and incubating at 95° C. for 5 minutes. After electrophoresis, proteins were transferred onto a nitrocellulose membrane using the Trans-Blot® Turbo™ Blotting System (Bio-Rad). The membranes were blocked for non-specific binding in 5% non-fatty dry milk in Tris Buffered Saline-Tween 0.1% (50 mM

Tris HCl ph 7.6, 150 mM NaCl, 0.1% Tween; TBS-T) for 1 h at room temperature (RT) and incubated with primary antibody overnight (ON) at 4° C. The following primary antibodies were used: Phospho-Met (Tyr1234/1235, Clone D26, Cell Signaling) 1:500 in TBS-T 5% BSA; Met (Clone D1C2, Cell Signaling) 1:1000 in TBS-T 5% BSA; Phospho-EGF Receptor (Tyr1068, Clone D7A5, Cell Signaling) 1:1000 in TBS-T 5% non-fatty dry milk; EGF Receptor (Clone D38B1, Cell Signaling) 1:1000 in TBS-T 5% BSA; Vinculin (Monoclonal anti-Vinculin, V9131, SIGMA Aldrich) 1:4000 in TBS-T 5% non-fatty dry milk. After incubation with the indicated primary antibodies, the membranes were washed for 15 minutes in TBS-T and incubated with secondary antibody (1:5000 in TBS-T 5% non-fatty dry milk) for 1 H at RT. The following secondary antibodies were used: goat anti-rabbit IgG-HRP (sc-2004, Santa Cruz biotechnology); goat anti-mouse IgG-HRP (sc-2005, Santa Cruz biotechnology). The signal was visualized with Enhanced Chemiluminescent Reagents (ECL; Invitrogen) or SuperSignal West Femto

Chemiluminescent Substrate (Thermo Scientific) with a digital imager (ImageQuant LAS 4000, GE Health Care Life Science Technologies).

FIG. 6 shows a Western blot analysis of the performed experiment.

In PC-9 cells, PB8532 and 5D5/cetuximab were able to reduce HGF induced phosphorylation. In addition both antibodies slightly reduced EGF phosphorylation in the absence and presence of EGF.

In HCC827 cells PB8532 reduced phosphorylation of cMET in the presence and absence of HGF. No effect was observed by the combination 5D5/cetuximab. Furthermore, in this cell line PB8532 reduced EGF induced phosphorylation of EGFR in contrast to the combination of 5D5 with cetuximab.

### Example 5

FIG. 7 depicts various sequences for alternative variable regions of the heavy chain of an EGFR binding variable domain as disclosed herein. FIG. 8 depicts various sequences for alternative variable regions of the heavy chain of a cMET binding variable domain as disclosed herein. The heavy chain variable regions were used to create a number of different cMET x EGFR bispecific antibodies. The light chain in these antibodies has the sequence as depicted in FIG. 9B. Bispecific antibodies were produced as described in example 1. The antibodies were also produced as an ADCC enhanced version. ADCC enhanced versions were produced by including in the co-transfection of the antibody constructs, a DNA encoding a reductase enzyme that removes a fucose residue from the Fc region of IgG1.

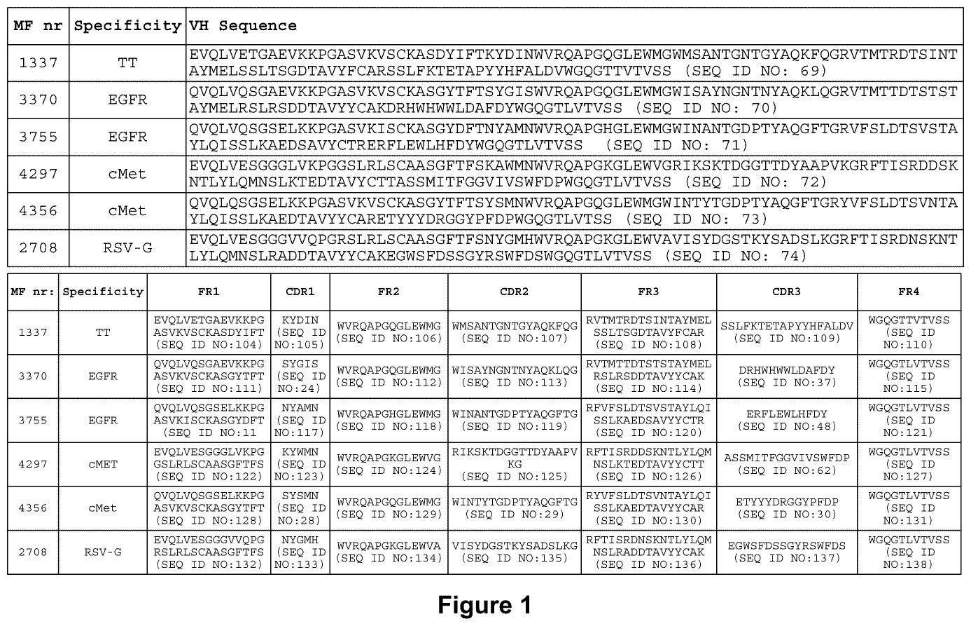

FIG. 17 depicts a titration of various produced bispecific antibodies on CHO-K1 EGFR cells described in example 2 (panel A) and on MKN-45 cells that endogenously express c-MET (panel B). The cells were incubated at 2×10⁵ cells/well with increasing concentrations of antibody as indicated. After washing, binding was detected with anti-human IgG-PE (3 μg/ml). Stained cells were analyzed on an iQue system and mean fluorescence intensity (MFI) and area under the curve (AUC) calculated. Control antibodies were MF1337xMF1337 (PG1337p218; TTxTT negative control; dark triangles at the bottom) and MF4356xMF3770 (PB8532p04; c-METxEGFR positive control; black triangles). TT stands for tetanus toxoid, A variable domain comprising the VH of MF1337 (see FIG. 1) and a common light chain as described herein, binds to Tetanus

Toxoid and is thus not expected to bind to the CHO-K1 EGFR cells and MKN-45 cells. The note (ADCC) indicates that the antibodies are produced with enhanced ADCC function through co-transfection with DNA encoding the RMD enzyme to remove a fucose residue from the Fc region of IgG1. See table 6 for a list of the bispecific antibodies used and their PB coding.

### ADCC Reporter Assay

An ADCC reporter assay was performed to determine whether co-transfection of RMD- encoding DNA successfully enhanced ADCC effector function. All samples were tested in duplo on BxPC3 cells (which express EGFR) and MKN-45 cells (which express c-MET) using both the high-affinity and low-affinity assay. The assay's high-affinity effector cells express the V-variant of the human FcyRIIIa and the low-affinity effector cells express the F-variant.

Briefly, the BxPC3 and MKN-45 target cells were harvested and plated at 1000 cells/well in 30 μL and incubated overnight at 37° C., 5% C02, 95% relative humidity. The next day, medium was removed and 10 μL antibody dilution added to each well (antibody dilution 1.5×; 9-step serial titration with semi-log dilution steps resulting in assay concentration of 1 ng/ml to 10 μg/ml). On the same day, effector cells were thawed at 37° C., and 630 μL added to 3.6 ml assay buffer in a 15-ml tube and mixed by inversion; 5 μL of this solution (15,000 cells) was then added to the wells of the assay plate. The plate was incubated for 6 hours at 37° C., 5% C02, 95% relative humidity before the addition of 15 μL Bio-Glo reagent to assay wells. Luminescence was measured using an EnVision plate reader.

A list of samples tested is provided in Table 6 and the results of the assays are provided in FIG. 18. The non-ADCC-enhanced anti-HER3 x EGFR control antibody (batch PB4522p25; MF4280xMF3178 described in W02015/130172) was negative in all four assays (indicated by black crosses and solid lines (4^{th} from above) in FIG. 18). In contrast, the ADCC-enhanced version of this antibody (PB4522p34) was positive in all four assays (indicated by solid orange circles). Similarly, the non-ADCC-enhanced anti-c-MET x EGFR control antibody (PB8532p04) was negative in all four assays (indicated by black crosses and dashed lines in FIG. 18), as was the PB8532p05 batch was also non-ADCC-enhanced (indicated by green asterisks). The three lines with asterisks are all at the bottom of the four panels. However, the ADCC-enhanced p06 variant (PB8532p06) was positive in all assays (indicated by open orange circles). Enhanced ADCC effector function similar to that of PB8532p06 was also seen for the 5 bispecifics (PB19474 to PB19478). This meant that co-transfection of RMD-encoding DNA successfully enhanced ADCC effector function.

### Example 6

The heavy chain variable region (VH) of the cMET variable domain of PB8532 comprises the amino acid of MF4356 as depicted for instance in FIG. 8. The VH of the cMET variable domain of PB19748 comprises the amino acid sequence of MF8230 (see FIG. 8). The VH of the EGFR variable domain of PB8532 comprises the amino acid of MF3370 as depicted for instance in FIG. 7. The VH of the EGFR variable domain of

PB19748 comprises the amino acid sequence of MF8233 of FIG. 7. The light chain in PB8532 and PB19748 is the same and is depicted in FIG. 9B. The cMET antibody LY2875358 antibody is among other described in Kim and Kim 2017. The capacity of the cMETxEGFR bispecific antibody PB8532 or PB19748 to inhibit tumor growth in vivo was tested alone and in combination with the receptor tyrosine kinase inhibitor erlotinib in a xenograft mouse model. In the chosen model, HCC827 tumor cells are engrafted into immunodeficient NOD SCID gamma (NSG) human hepatocyte growth factor knock-in (hHGFki) mice, that express human HGF (ligand for cMET) in place of endogenous mouse HGF. The NSG-hHGFki mice are known in full as NOD.Cg-Hgf^{tm1.1(HGF)Aveo} Prk^{dcscid I12rgtm1Wjl/J} (stk#014553) (NOD.Cg-Hgftm1.1(HGF)Aveo Prkdcscid I12rgtm1Wjl/J).

They have no T or B cells, lack functional NK cells, and are deficient in cytokine signaling, which allows for better tumor engraftment. HCC827 is an established human non-small-cell lung carcinoma (NSCLC) cell line that expresses EGFR and cMET and is known to be resistant to erlotinib in the presence of HGF.

To establish the effect of erlotinib on tumor growth in this model, a first experiment was done with two groups of mice. Prior to tumor cell engraftment, the cell cycle of HCC827 cells was boosted by culturing the cells overnight in medium supplemented with 20% fetal bovine serum (FBS) at confluency not exceeding 80%. The next day, NSG-hHGFki mice (The Jackson Laboratory) were inoculated subcutaneously with 17×10e6 HCC827 tumor cells suspended in 300 μl PBS plus 30% matrigel containing a high concentration of basement membrane matrix. The resulting tumors were measured twice each week using calipers. When the mean tumor volume reached approximately 200 mm³, the mice were randomized into two groups (4-7 mice per group depending on tumor growth and volume) and drug treatment was started.

A fine suspension of erlotinib was prepared freshly every week in 0.05% hydroxypropyl methylcellulose (HPMC) and 0.2% Tween-80 in water by sonication.

From day 19, the erlotinib solution was used to treat 5 mice once daily (QD) via gavage at a dose of 6 mg/kg (n=5), and a group of 4 mice was given once daily gavage with 200 ηl vehicle (0.05% HPMC and 0.1% Tween 80 in water). Tumor volume was measured twice each week using calipers, and mean tumor volume (and SEM) calculated for each group. When tumor sizes reached 1500 mm³ mice were euthanized. Treatment was stopped after day 48, and tumor volume in surviving mice was measured up to day 62.

In a second experiment that tested the capacity of the PB8532 bispecific antibody to inhibit tumor growth in this model (alone and in combination with erlotinib), NSG-hHGFki mice with tumors (generated as described above) were given one of six treatments or combination treatments, whereby antibody was given weekly by intraperitoneal (i.p.) injection and erlotinib or vehicle was given once a day (QD) by gavage. As a negative control, mice were also treated with PB17160, a bispecific antibody made up of the same anti-cMET Fab arm as in PB8532, in combination with a Fab arm specific for an irrelevant target. An irrelevant target is for instance a target that is not present in the mouse and tumor. Often a tetanus toxoid specific variable domain is used. A suitable tetanus toxoid binding variable domain has the VH of MF1337 (see FIG. 1) and a common light as disclosed herein, preferably a sequence of FIG. 9. Bispecific antibodies with a targeting arm and a non-targeting (TT) arm are among others described in W02017/069628, see MF1337, which is incorporated by reference herein. Another irrelevant target is RSV-G. A suitable RSV-G variable domain has the VH of MF2708 of FIG. 1 and a common light chain, preferably one of FIG. 9, preferably 9B.

On day 21, when the mean tumor volume had reached approximately 200 mm³, the mice were randomized into six groups (5-7 mice per group depending on tumor growth and volume) and drug treatment was started: daily gavage with vehicle alone (n=5); weekly i.p. injections of 25 mg/kg PB8532 antibody plus daily gavage with vehicle (n=7); weekly i.p. injections of 25 mg/kg PB17160 antibody plus daily oral gavage with vehicle (n=6); daily oral gavage with 6 mg/kg erlotinib (n=7); weekly i.p. injections of 25 mg/kg PB8532 antibody plus daily oral gavage with 6 mg/kg erlotinib (n=7); or weekly i.p. injections of 25 mg/kg PB17160 antibody plus daily oral gavage with 6 mg/kg erlotinib (n=7). As before, tumor volume was monitored and mean tumor volume (and SEM) calculated in each group. All treatments were stopped after day 60, and tumor volume in surviving mice was measured up to day 82.

In a third experiment the capacity of the PB19478 bispecific antibody to inhibit tumor growth in this model (alone and in combination with erlotinib) was tested. NSG-hHGFki mice with tumors (generated as described above) were given one of six treatments or combination treatments, whereby antibody was given weekly by intraperitoneal (i.p.) injection and erlotinib or vehicle was given once a day (QD) by gavage.

On day 23, when the mean tumor volume had reached approximately 200 mm³, the mice were randomized into six groups (5-6 mice per group depending on tumor growth and volume) and drug treatment was started: daily gavage with vehicle alone (n=5); weekly i.p. injections of 25 mg/kg PB19478 antibody plus daily gavage with vehicle (n=5); daily oral gavage with 6 mg/kg erlotinib (n=6); weekly i.p. injections of 25 mg/kg PB19478 antibody plus daily oral gavage with 6 mg/kg erlotinib (n=4, one mouse died during the experiment); weekly i.p. injections of 25 mg/kg LY2875358 antibody plus daily gavage with vehicle (n=5); weekly i.p. injections of 25 mg/kg LY2875358 antibody plus daily oral gavage with 6 mg/kg erlotinib (n=3, two mice died during the experiment). As before, tumor volume was monitored and mean tumor volume (and SEM) calculated in each group. All treatments were stopped after day 93, and tumor volume in surviving mice was measured up to day 93.

In a fourth experiment the effect of a later administration of PB19478 was tested. NSG-hHGFki mice with tumors (generated as described above) were given one of two treatments, whereby antibody was given weekly by intraperitoneal (i.p.) injection and erlotinib or vehicle was given once a day (QD) by gavage.

On day 21, when the mean tumor volume had reached approximately 200 mm³, all 14 mice were started on a daily oral gavage treatment with 6 mg/kg erlotinib. On day 51 when the mean tumor volume had clearly passed the 500 mm 3 mark the mice were randomized into two groups. One group of six were treated with daily oral gavage with 6 mg/kg erlotinib and a group of 8 received weekly i.p. injections of 25 mg/kg PB19478 antibody plus daily gavage with 6 mg/kg erlotinib. As before, tumor volume was monitored and mean tumor volume (and SEM) calculated in each group. All treatments were stopped after day 72.

The results of the first experiment demonstrate that erlotinib was able to induce an anti-tumor response in NGS-hHGFki mice engrafted with HCC827 cells, but only for as long as the mice were receiving treatment (FIG. 19). In the drug-free period that commenced after about 4 weeks of treatment, tumor volume clearly increased in the mice that had been treated with erlotinib.

The anti-cMETxEGFR bispecific antibody PB8532 was also able to induce an anti-tumor response in NGS-hHGFki mice engrafted with HCC827 cells (FIG. 20). This effect was greater when the antibody was given in combination with daily doses of erlotinib. Within 2.5 weeks all tumors disappeared from the combination of PB8532 with erlotinib. The control antibody PB17160 targeting cMet with one Fab arm induced no anti-tumor response, either with or without erlotinib. Thus, the specific targeting of the cMet Fab arm by combination with an EGFR targeting Fab arm in bispecific antibody PB8532 can overcome HGF mediated erlotinib resistance.

In the drug-free period that commenced after about 5½ weeks of treatment, PB8532 was clearly more effective than erlotinib in reducing tumor volume (FIG. 21) and no tumor regrowth was observed in the PB8532+erlotinib combination group.

The anti-cMETxEGFR bispecific antibody PB19478 was also able to induce an anti-tumor response in NGS-hHGFki mice engrafted with HCC827 cells (FIG. 22). This effect was greater when the antibody was given in combination with daily doses of erlotinib. Within 2 weeks all tumors disappeared from the combination of PB19478 with or without erlotinib. With erlotinib the tumor did not reappear in the test period. Without erlotinib the tumor reappeared shortly around day 50 and stayed at the detection level until it finally grew further at day 80 onwards. The humanized monoclonal antibody emibetuzumab (LY3875358) was less effective, also when combined with the EGFR inhibitor erlotinib. Thus, the specific targeting of the cMet Fab arm by combination with an EGFR targeting Fab arm in bispecific antibody PB8532 or PB19478 can overcome HGF mediated erlotinib resistance.

FIG. 23 shows that when you treat tumors at a time point where erlotinib resistance starts to develop there is an immediate effect of the bispecific antibody PB19478.

Taken together, the data from this xenograft model of HCC827 tumor cells engrafted into immunodeficient NSG-hHGFki mice show that PB8532, PB19478 and antibodies having the similar VH sequences given in FIGS. 7 and 8 have the capacity to overcome HGF-mediated erlotinib resistance in vivo. The combination treatment continues to be effective after stopping treatment.

### Cited Art

Ahmed, M et al. Lack of in Vivo Antibody Dependent Cellular Cytotoxicity with Antibody containing gold particles/ Bioconjugate chemistry (2015): 26 812-816. DOI: 10.1021/acs.bioconjchem.5b00139.
Buday L, Downward J. Epidermal growth factor regulates p21ras through the formation of a complex of receptor, Grb2 adapter protein, and Sos nucleotide exchange factor. Cell 1993;73: 611-20.
Castoldi, R., et al. “A novel bispecific EGFR/Met antibody blocks tumor-promoting phenotypic effects induced by resistance to EGFR inhibition and has potent antitumor activity.” Oncogene 32.50 (2013): 5593-5601.
Chen et al., MET activation mediates resistance to lapatinib inhibition of HER2-amplified gastric cancer cells. Mol Cancer Thor 2012;11(3)650-669
Ferguson K M. Structure-based view of epidermal growth factor receptor regulation. Annu Rev Biophys 2008;37: 353-73.
Fong et al., Alternative signaling pathways as potential therapeutic targets for overcoming EGFR and c-Met inhibitor resistance in non-small cell lung cancer. PLoS One. 2013 Nov 4;8(11):e78398.
Gale NW, Kaplan S, Lowenstein E J, Schlessinger J, Bar-Sagi D. Grb2 mediates the EGF-dependent activation of guanine nucleotide exchange on Ras. Nature 1993; 363:88-92.
Gulli LF1, Palmer K C, Chen Y Q, Reddy K B. 1996. Epidermal growth factor-induced apoptosis in A431 cells can be reversed by reducing the tyrosine kinase activity. Cell Growth Differ. Feb;7(2)173-8.
Jorissen R N, Walker F, Pouliot N, Garrett T P, Ward C W, Burgess A W. Epidermal growth factor receptor: mechanisms of activation and signalling. Exp Cell Res 2003;284:31-53.
Kim, George P., and Axel Grothey. “Targeting colorectal cancer with human anti-EGFR monoclonal antibodies: focus on panitumumab.” Biologics 2.2 (2008): 223-228.
Kim, Ki-Hyun and Kim, Hyori. Progress of antibody-based inhibitors of the HGF-cMET axis in cancer therapy. Experimental & Molecular medicine (2017), e307; doi:10.1038/emm 2017.17)
Morgillo F, et al. ESMO Open 2016;1:e000060. doi:10.1136/esmoopen-2016-000060
Moores, Sheri L., et al. “A novel bispecific antibody targeting EGFR and cMet is effective against EGFR inhibitor—resistant lung tumors.” Cancer research 76.13 (2016): 3942-3953.
Nakade et al. Triple inhibition of EGFR, Met, and VEGF suppresses regrowth of HGF-triggered, erlotinib-resistant lung cancer harboring an EGFR mutation. J Thorac Oncol 2014, 9(6)775-83.
Prigent S A, Gullick W J. Identification of cErbB-3 binding sites for phosphatidylinositol 30-kinase and SHC using an EGF receptor/c-ErbB-3 chimera. EMBO J 1994;13:2831-41.
Robertson S C, Tynan J, Donoghue D J. RTK mutations and human syndromes: when good receptors turn bad. Trends Genet 2000;16:368.
Soltoff S P, Carraway K L, III, Prigent S A, Gullick W G, Cantley LC. ErbB3 is involved in activation of phosphatidylinositol 3-kinase by epidermal growth factor. Mol Cell Biol 1994;14:3550-8.
Yano et al. Hepatocyte growth factor induces gefitinib resistance of lung adenocarcinoma with epidermal growth factor receptor-activating mutations. Cancer Res. 2008, 15;68(22):9479-87.
Yarden Y. The EGFR family and its ligands in human cancer. Signalling mechanisms and therapeutic opportunities. Eur J Cancer 2001;37 (Suppl 4):S3—S8.
Zhang et al., MET kinase inhibitor SGX₅₂₃ synergizes with epidermal growth factor receptor inhibitor erlotinib in a hepatocyte growth factor-dependent fashion to suppress carcinoma growth. Cancer Res 2010, 70:6880-6890.
US20120107234A1_Symphogen patent “PAN HER antibody composition”. 125084 Erbitux Pharmacology Review Part 2—FDA.

TABLE 1
Reference antibodies with reported specificities
against cMET extracellular domains.

| Name | INN name | Epitope | MOA |
|---|---|---|---|
| 5D5 | MetMAb | Sema Domain | HGF block |
| 13.3.2 | | | |
| 224G11 | | | |
| C8-H241 | LY-2875358 | | HGF block, |
| | | | internalization |
| R13 | 13-MET | | |

TABLE 2
Competition of cMet reference antibodies with cMET
cLC antibodies. Shown are OD450 values. OD450 values
indicate the existence or lack of competition with
the stated antibody. MF4506 was not tested.

| | | Competition of phages with reference antibodies | | | | | |
|---|---|---|---|---|---|---|---|
| MF tested | no IgG | 13.3.2 | 5D5 | R13 | 224G11 | C8H241 | R28 |
| 4040 | 1.915 | 1.818 | 0.066 | 1.608 | 1.907 | 1.979 | 1.787 |
| 4297 | 1.769 | 0.072 | 1.499 | 1.332 | 1.955 | 1.031 | 1.885 |
| 4356 | 2.380 | 2.541 | 0.088 | 2.231 | 2.170 | 1.806 | 1.825 |
| 13.3.2 | 2.172 | 0.311 | 1.934 | 1.988 | 2.221 | 1.893 | 2.129 |
| 5D5 | 1.868 | 1.773 | 0.164 | 1.660 | 2.025 | 2.054 | 2.035 |
| R13 | 1.693 | 1.590 | 1.549 | 0.090 | 1.878 | 0.078 | 1.794 |

TABLE 3
List of the 24 cMETxEGFR bispecifics antibodies selected
after dose dependent titration experiments in a N87
HGF/EGF proliferation assay. The MF number of the EGFR
and cMET arms in each individual PB as well as
the their HCDR3 sequence are indicated.

| | PBs MF's | | | |
|---|---|---|---|---|
| PBs | EGFR | | cMET | |
| PB7678 | MF4280 | EGYYETTTYYYNLFDS | MF4298 | KLEPTGYYYYMDV |
| | | (SEQ ID NO: 47) | | (SEQ ID NO: 52) |
| PB7679 | MF3755 | ERFLEWLHFDY | MF4487 | KTSRYSGYHYYMDV |
| | | (SEQ ID NO: 48) | | (SEQ ID NO: 53) |
| PB7686 | MF3755 | ERFLEWLHFDY | MF4507 | AHYDILTG |
| | | (SEQ ID NO: 48) | | (SEQ ID NO: 54) |
| PB8021 | MF3755 | ERFLEWLHFDY | MF3462 | GKSHYSWDAFDY |
| | | (SEQ ID NO: 48) | | (SEQ ID NO: 55) |
| PB8218 | MF3752 | DRNWGWDFDY | MF4040 | GTYYYGSGSFSTRVFDAFDV |
| | | (SEQ ID NO: 49) | | (SEQ ID NO: 56) |
| PB8244 | MF3755 | ERFLEWLHFDY | MF4044 | QSRRYSGYASYFDY |
| | | (SEQ ID NO: 48) | | (SEQ ID NO: 57) |
| PB8292 | MF3755 | ERFLEWLHFDY | MF4130 | QRRAYSGYNWYFDL |
| | | (SEQ ID NO: 48) | | (SEQ ID NO: 58) |
| PB8301 | MF4280 | EGYYETTTYYYNLFDS | MF4130 | QRRAYSGYNWYFDL |
| | | (SEQ ID NO: 47) | | (SEQ ID NO: 58) |
| PB8316 | MF3755 | ERFLEWLHFDY | MF4293 | RNDFWSGYLFDY |
| | | (SEQ ID NO: 48) | | (SEQ ID NO: 59) |
| PB8339 | MF3752 | DRNWGWDFDY | MF4294 | KTTVGYYYYYMDV |
| | | (SEQ ID NO: 49) | | (SEQ ID NO: 60) |
| PB8340 | MF3755 | ERFLEWLHFDY | MF4294 | KTTVGYYYYYMDV |
| | | (SEQ ID NO: 48) | | (SEQ ID NO: 60) |
| PB8364 | MF3755 | ERFLEWLHFDY | MF4296 | GPELGYYYYYMDI |
| | | (SEQ ID NO: 48) | | (SEQ ID NO: 61) |
| PB8388 | MF3755 | ERFLEWLHFDY | MF4297 | ASSMITFGGVIVSWFDP |
| | | (SEQ ID NO: 48) | | (SEQ ID NO: 62) |
| PB8511 | MF3755 | ERFLEWLHFDY | MF4301 | RVNRYSGYATYFDL |
| | | (SEQ ID NO: 48) | | (SEQ ID NO: 63) |
| PB8532 | MF3370 | DRHWHWWLDAFDY | MF4356 | ETYYYDRGGYPFDP |
| | | (SEQ ID NO: 37) | | (SEQ ID NO: 30) |
| PB8535 | MF3755 | ERFLEWLHFDY | MF4356 | ETYYYDRGGYPFDP |
| | | (SEQ ID NO: 48) | | (SEQ ID NO: 30) |
| PB8545 | MF4281 | GDLFITGTLDY | MF4356 | ETYYYDRGGYPFDP |
| | | (SEQ ID NO: 51) | | (SEQ ID NO: 30) |
| PB8582 | MF3752 | DRNWGWDFDY | MF4491 | RTSRYSGYHYYLDV |
| | | (SEQ ID NO: 49) | | (SEQ ID NO: 65) |
| PB8583 | MF3755 | ERFLEWLHFDY | MF4491 | RTSRYSGYHYYLDV |
| | | (SEQ ID NO: 48) | | (SEQ ID NO: 65) |
| PB8607 | MF3755 | ERFLEWLHFDY | MF4505 | LLYDLFDL |
| | | (SEQ ID NO: 48) | | (SEQ ID NO: 66) |
| PB8639 | MF3752 | DRNWGWDFDY | MF4506 | SIDMATITDAFDI |
| | | (SEQ ID NO: 49) | | (SEQ ID NO: 67) |
| PB8640 | MF3755 | ERFLEWLHFDY | MF4506 | SIDMATITDAFDI |
| | | (SEQ ID NO: 48) | | (SEQ ID NO: 67) |
| PB8687 | MF3752 | DRNWGWDFDY | MF4508 | GTTGNPYYFYYYMDV |
| | | (SEQ ID NO: 49) | | (SEQ ID NO: 68) |
| PB8688 | MF3755 | ERFLEWLHFDY | MF4508 | GTTGNPYYFYYYMDV |
| | | (SEQ ID NO: 48) | | (SEQ ID NO: 68) |

TABLE 4
Summary of the antibody titration experiments done using
N87 HGF/EGF, HGF and EGF proliferation assays with the 24
cMETxEGFR bispecific antibodies. Bispecifics are indicated
as PBXXXX and the different Fab arms with MGXXXX. The activity
of the bispecifics in the individual assays is indicated
as: − no effect; + inhibition, of proliferation
lower than positive control; ++ = inhibition
of proliferation comparable to positive control antibody
5D5 Fab; +++ = Inhibition of proliferation
higher than positive control antibody 5D5 Fab.

| | | | N87 proliferation assays | | |
|---|---|---|---|---|---|
| | PBs MF's | | HGF/ | | |
| PBs | EGFR | cMET | EGF | HGF | EGF |
| PB7678 | MF4280 | MF4298 | + | + | + |
| PB7679 | MF3755 | MF4487 | ++ | − | ++ |
| PB7686 | MF3755 | MF4507 | ++ | + | + |
| PB8021 | MF3755 | MF3462 | + | + | + |
| PB8218 | MF3732 | MF4040 | ++ | +++ | + |
| PB8244 | MF3755 | MF4044 | ++ | + | ++ |
| PB8292 | MF3755 | MF4130 | ++ | + | ++ |
| PB8301 | MF4280 | MF4130 | + | ++ | + |
| PB8316 | MF3755 | MF4293 | ++ | + | + |
| PB8339 | MF3752 | MF4294 | + | + | + |
| PB8340 | MF3755 | MF4294 | ++ | + | ++ |
| PB8364 | MF3755 | MF4296 | ++ | + | ++ |
| PB8388 | MF3755 | MF4297 | ++ | +++ | ++ |
| PB8511 | MF3755 | MF4301 | ++ | +++ | ++ |
| PB8532 | MF3370 | MF4358 | + | +++ | + |
| PB8535 | MF3755 | MF4358 | ++ | +++ | ++ |
| PB8545 | ME4281 | MF4358 | + | +++ | + |
| PB8582 | MF3752 | MF4491 | + | − | + |
| PB8583 | MF3755 | MF4491 | ++ | +++ | ++ |
| PB8607 | MF3755 | MF4505 | ++ | − | ++ |
| PB8639 | MF3752 | MF4506 | + | +++ | + |
| PB8640 | MF3755 | MF4508 | ++ | +++ | ++ |
| PB8687 | MF3752 | MF4508 | + | ++ | + |
| PB8688 | MF3755 | MF4508 | + | − | + |

TABLE 5
Composition of the most potent EGFRxcMET bispecific antibodies
and their competition with reference antibodies.

| | | EGFE | | |
|---|---|---|---|---|
| | | blocking | | |
| | | compared | | Cross |
| | | to cetux | | reference |
| Bispecific | | (based on | cMET | antibody |
| antibody | EGFR arm | IC50) | arm | blocking |
| PB8535 | MF3753 | 100% | MF4358 | 5D5 |
| PB8640 | MF3755 | 100% | MF4506 | ND |
| PB8388 | MF3755 | 100% | MF4297 | 13.3.2 |
| PB8218 | MF3752 | 80% | MF4040 | 5D5 |
| PB8532 | MF3370 | 80% | MF4358 | 5D5 |

TABLE 6
Composition of bispecific antibodies. The pXX number indicates
the number of the production run and can be used to identify
whether the antibody was produced in an ADCC version or not.

| Bispecific antibody | cMET arm | EGFE arm | ADCC enhanced |
|---|---|---|---|
| Cetuximab | — | — | — |
| PB8532p05 | MF4356 | MF3370 | No |
| PB19474p01 | MF4356 | MF8232 | Yes |
| PB19475p01 | MF4356 | MF8233 | Yes |
| PB19476p01 | MF8230 | MF3370 | Yes |
| PB19477p01 | MF8230 | MF8232 | Yes |
| PB19478p01 | MF8230 | MF8233 | Yes |
| PB8532p06 | MF4358 | MF3370 | Yes |
| PB8532p04 | MF4356 | MF3370 | No |
| | HER-3 arm | EGFR arm | |
| PB4522p34 | MF3178 | MF4280 | Yes |
| PB4522p25 | MF3178 | MF4280 | No |
| | TT arm | TT arm | |
| PG1337p218 | MF1337 | MF1337 | No |

1-45. (canceled)

46. A method of treating a subject that has a tumor or a disease involving aberrant cells, the method comprising administering to an individual in need thereof a bispecific antibody that comprises a first variable domain that can bind an extracellular part of human epidermal growth factor receptor (EGFR) and a second variable domain that can bind an extracellular part of human MET Proto-Oncogene, Receptor Tyrosine Kinase (cMET);
wherein the first variable domain comprises a heavy chain variable region with a CDR1 sequence SYGIS (SEQ ID NO: 24), a CDR2 sequence WISAYNANTNYAQKLQG (SEQ ID NO:155), and a CDR3 comprising the sequence DRHWHWWLDA (SEQ ID NO:139), and wherein the second variable domain comprises a heavy chain variable region with a CDR1 sequence TYSMN (SEQ ID NO: 152), a CDR2 sequence WINTYTGDPTYAQGFTG (SEQ ID NO:157), and a CDR3 comprising the sequence ETYFYDRGGYPFDP (SEQ ID NO: 31);
wherein the first variable domain comprises a heavy chain variable region with a CDR1 sequence SYGIS (SEQ ID NO: 24), a CDR2 sequence WISAYNANTNYAQKLQG (SEQ ID NO:155), and a CDR3 comprising the sequence DRHWHWWLDAFDY (SEQ ID NO:37), and wherein the second variable domain comprises a heavy chain variable region with a CDR1 sequence TYSMN (SEQ ID NO: 152), a CDR2 sequence WINTYTGDPTYAQGFTG (SEQ ID NO:157), and a CDR3 comprising the sequence ETYFYDRGGYPFDP (SEQ ID NO: 31);
wherein the first variable domain comprises a heavy chain variable region with a CDR1 sequence SYGIS (SEQ ID NO: 24), a CDR2 sequence WISAYSGNTNYAQKLQG (SEQ ID NO:140), and a CDR3 comprising the sequence DRHWHWWLDA (SEQ ID NO:139), and wherein the second variable domain comprises a heavy chain variable region with a CDR1 sequence TYSMN (SEQ ID NO: 152), a CDR2 sequence WINTYTGDPTYAQGFTG (SEQ ID NO:157), and a CDR3 comprising the sequence ETYFYDRGGYPFDP (SEQ ID NO: 31);
wherein the first variable domain comprises a heavy chain variable region with a CDR1 sequence SYGIS (SEQ ID NO: 24), a CDR2 sequence WISAYSGNTNYAQKLQG (SEQ ID NO:140), and a CDR3 comprising the sequence DRHWHWWLDA (SEQ ID NO:139), and wherein the second variable domain comprises a heavy chain variable region with a CDR1 sequence SYSMN (SEQ ID NO: 28), a CDR2 sequence WINTYTGDPTYAQGFTG (SEQ ID NO: 157), and a CDR3 sequence ETYYYDRGGYPFDP (SEQ ID NO: 30);
wherein the first variable domain comprises a heavy chain variable region with a CDR1 sequence SYGIS (SEQ ID NO: 24), a CDR2 sequence WISAYSGNTNYAQKLQG (SEQ ID NO:140), and a CDR3 comprising the sequence DRHWHWWLDAFDY (SEQ ID NO:37), and wherein the second variable domain comprises a heavy chain variable region with a CDR1 sequence SYSMN (SEQ ID NO: 28), a CDR2 sequence WINTYTGDPTYAQGFTG (SEQ ID NO: 157), and a CDR3 sequence ETYYYDRGGYPFDP (SEQ ID NO: 30);
wherein the first variable domain comprises a heavy chain variable region with a CDR1 sequence SYGIS (SEQ ID NO: 24), a CDR2 sequence WISAYNANTNYAQKLQG (SEQ ID NO:155), and a CDR3 comprising the sequence DRHWHWWLDAFDY (SEQ ID NO: 37), and wherein the second variable domain comprises a heavy chain variable region with a CDR1 sequence SYSMN (SEQ ID NO: 28), a CDR2 sequence WINTYTGDPTYAQGFTG (SEQ ID NO: 157), and a CDR3 sequence ETYYYDRGGYPFDP (SEQ ID NO: 30);
wherein the first variable domain comprises a heavy chain variable region with a CDR1 sequence SYGIS (SEQ ID NO: 24), a CDR2 sequence WISAYNGNTNYAQKLQG (SEQ ID NO:113), and a CDR3 comprising the sequence DRHWHWWLDAFDY (SEQ ID NO: 37), and wherein the second variable domain comprises a heavy chain variable region with a CDR1 sequence TYSMN (SEQ ID NO: 152), a CDR2 sequence WINTYTGDPTYAQGFTG (SEQ ID NO:157), and a CDR3 comprising the sequence ETYFYDRGGYPFDP (SEQ ID NO: 31); or
wherein the first variable domain comprises a heavy chain variable region with a CDR1 sequence SYGIS (SEQ ID NO: 24), a CDR2 sequence WISAYNGNTNYAQKLQG (SEQ ID NO:113), and a CDR3 comprising the sequence DRHWHWWLDAFDY (SEQ ID NO: 37), and wherein the second variable domain comprises a heavy chain variable region with a CDR1 sequence SYSMN (SEQ ID NO: 28), a CDR2 sequence WINTYTGDPTYAQGFTG (SEQ ID NO: 157), and a CDR3 sequence ETYYYDRGGYPFDP (SEQ ID NO: 30); and
wherein the first and second variable domains further comprise a common light chain variable domain comprising a CDR1 sequence QSISSY (SEQ ID NO:38), a CDR2 sequence AAS, and a CDR3 sequence QQSYSTP (SEQ ID NO: 39).

47. The method of claim 46, wherein the tumor is an EGFR positive tumor, a cMET positive tumor, or an EGFR and cMET positive tumor.

48. The method of claim 46, wherein the disease is a breast cancer, colon cancer, pancreatic cancer, gastric cancer, ovarian cancer, colorectal cancer, head and neck cancer, lung cancer including non-small cell lung cancer, or bladder cancer.

49. The method of claim 46, wherein the disease is a lung cancer including non-small cell lung cancer.

50. The method of claim 46, wherein the tumor or the aberrant cells are resistant to treatment with an EGFR tyrosine kinase inhibitor.

51. The method of claim 50, wherein the EGFR tyrosine kinase inhibitor is erlotinib, gefitinib, or afatinib, an analogue of erlotinib, gefitinib, or afatinib, or a combination of one or more of the respective compounds and/or analogues thereof.

52. The method of claim 50, wherein the EGFR tyrosine kinase inhibitor is erlotinib.

53. The method of claim 46, wherein the treatment further comprises administering an EGFR tyrosine kinase inhibitor to the individual in need thereof.

54. The method of claim 53, wherein the EGFR tyrosine kinase inhibitor is erlotinib.

55. The method of claim 53, wherein the said bispecific antibody is administered simultaneously, sequentially or separately with said EGFR tyrosine kinase inhibitor.

56. The method of claim 46, that is a IgG1 format antibody having an anti-EGFR, anti-cMET stoichiometry of 1:1.

57. The method of claim 46, wherein the amino acids at positions 405 and 409 in one CH3 domain are the same as the amino acids at the corresponding positions in the other CH3 domain (EU-numbering).

58. The method of claim 46, wherein the heavy chain variable region of the second variable domain comprises the amino acid sequence of one of the sequences of SEQ ID NO: 1-3, 7, 8, 10, 13, 15, 16, 17, 21, 22, and 23.

59. The method of claim 46, wherein the heavy chain variable region of the second variable domain comprises the amino acid sequence of one of the sequences of SEQ ID NO: 2, 7, 8, 10, 13, and 23.

60. The method of claim 46, wherein the first variable domain comprises a heavy chain variable region with a CDR1 sequence SYGIS (SEQ ID NO: 24), a CDR2 sequence WISAYNANTNYAQKLQG (SEQ ID NO:155), and a CDR3 comprising the sequence DRHWHWWLDA (SEQ ID NO:139), and wherein the second variable domain comprises a heavy chain variable region with a CDR1 sequence TYSMN (SEQ ID NO: 152), a CDR2 sequence WINTYTGDPTYAQGFTG (SEQ ID NO:157), and a CDR3 comprising the sequence ETYFYDRGGYPFDP (SEQ ID NO: 31).

61. The method of claim 46, wherein the first variable domain comprises a heavy chain variable region with a CDR1 sequence SYGIS (SEQ ID NO: 24), a CDR2 sequence WISAYNANTNYAQKLQG (SEQ ID NO:155), and a CDR3 comprising the sequence DRHWHWWLDAFDY (SEQ ID NO: 37), and wherein the second variable domain comprises a heavy chain variable region with a CDR1 sequence TYSMN (SEQ ID NO: 152), a CDR2 sequence WINTYTGDPTYAQGFTG (SEQ ID NO:157), and a CDR3 comprising the sequence ETYFYDRGGYPFDP (SEQ ID NO: 31).

62. The method of claim 46, wherein the first variable domain comprises a heavy chain variable region with a CDR1 sequence SYGIS (SEQ ID NO: 24), a CDR2 sequence WISAYSGNTNYAQKLQG (SEQ ID NO: 140), and a CDR3 comprising the sequence DRHWHWWLDA (SEQ ID NO: 139), and wherein the second variable domain comprises a heavy chain variable region with a CDR1 sequence TYSMN (SEQ ID NO: 152), a CDR2 sequence WINTYTGDPTYAQGFTG (SEQ ID NO:157), and a CDR3 comprising the sequence ETYFYDRGGYPFDP (SEQ ID NO: 31).

63. The method of claim 46, wherein the first variable domain comprises a heavy chain variable region with a CDR1 sequence SYGIS (SEQ ID NO: 24), a CDR2 sequence WISAYSGNTNYAQKLQG (SEQ ID NO: 140), and a CDR3 comprising the sequence DRHWHWWLDA (SEQ ID NO: 139), and wherein the second variable domain comprises a heavy chain variable region with a CDR1 sequence SYSMN (SEQ ID NO: 28), a CDR2 sequence WINTYTGDPTYAQGFTG (SEQ ID NO:157), and a CDR3 sequence ETYYYDRGGYPFDP (SEQ ID NO: 30).

64. The method of claim 46, wherein the first variable domain comprises a heavy chain variable region comprising the amino acid sequence of SEQ ID NO:79 and the second variable domain comprises the amino acid sequence of SEQ ID NO:13.

65. The method of claim 46, wherein the first variable domain comprises a heavy chain variable region comprising the amino acid sequence of SEQ ID NO:80 and the second variable domain comprises the amino acid sequence of SEQ ID NO:13.

66. The method of claim 46, wherein the first variable domain comprises a heavy chain variable region comprising the amino acid sequence of SEQ ID NO:76 and the second variable domain comprises the amino acid sequence of SEQ ID NO:13.

67. The method of claim 46, wherein the first variable domain comprises a heavy chain variable region with a CDR1 sequence SYGIS (SEQ ID NO: 24), a CDR2 sequence WISAYSGNTNYAQKLQG (SEQ ID NO:140), and a CDR3 comprising the sequence DRHWHWWLDAFDY (SEQ ID NO:37), and wherein the second variable domain comprises a heavy chain variable region with a CDR1 sequence SYSMN (SEQ ID NO: 28), a CDR2 sequence WINTYTGDPTYAQGFTG (SEQ ID NO: 157), and a CDR3 sequence ETYYYDRGGYPFDP (SEQ ID NO: 30).

68. The method of claim 67, wherein the first variable domain comprises a heavy chain variable region with a CDR1 sequence SYGIS (SEQ ID NO: 24), a CDR2 sequence WISAYNANTNYAQKLQG (SEQ ID NO:155), and a CDR3 comprising the sequence DRHWHWWLDAFDY (SEQ ID NO: 37), and wherein the second variable domain comprises a heavy chain variable region with a CDR1 sequence SYSMN (SEQ ID NO: 28), a CDR2 sequence WINTYTGDPTYAQGFTG (SEQ ID NO: 157), and a CDR3 sequence ETYYYDRGGYPFDP (SEQ ID NO: 30).

69. The method of claim 46, wherein the first variable domain comprises a heavy chain variable region with a CDR1 sequence SYGIS (SEQ ID NO: 24), a CDR2 sequence WISAYNGNTNYAQKLQG (SEQ ID NO:113), and a CDR3 comprising the sequence DRHWHWWLDAFDY (SEQ ID NO: 37), and wherein the second variable domain comprises a heavy chain variable region with a CDR1 sequence TYSMN (SEQ ID NO: 152), a CDR2 sequence WINTYTGDPTYAQGFTG (SEQ ID NO:157), and a CDR3 comprising the sequence ETYFYDRGGYPFDP (SEQ ID NO: 31).

70. The method of claim 46, wherein the first variable domain comprises a heavy chain variable region with a CDR1 sequence SYGIS (SEQ ID NO: 24), a CDR2 sequence WISAYNGNTNYAQKLQG (SEQ ID NO:113), and a CDR3 comprising the sequence DRHWHWWLDAFDY (SEQ ID NO: 37), and wherein the second variable domain comprises a heavy chain variable region with a CDR1 sequence SYSMN (SEQ ID NO: 28), a CDR2 sequence WINTYTGDPTYAQGFTG (SEQ ID NO: 157), and a CDR3 sequence ETYYYDRGGYPFDP (SEQ ID NO: 30).


SEQUENCE LISTING (159 sequences)

SEQ ID NO: 1 - protein, 123 aa, synthetic construct
  REGION 1..123: note = MF8225
QVQLVQSGSELKKPGASVKVSCKASGYTFTTYSLNWVRQAPGQGLEWMGWINTYTGNPTYAQGFTGRFVFFLDTSVSTAYLQISSLKAEDTAVYYCARETYYYDSSGYPFDPWGQGTLVTVSS

SEQ ID NO: 2 - protein, 123 aa, synthetic construct
  REGION 1..123: note = MF8243
QVQLVQSGSELKKPGASVKVSCKASGYTFTSYAMNWVRQAPGQGLEWMGWINTNTGNPTYAQGFTGRFVFSLDTSVSTAYLQISSLKAEDTAVYYCARETYYYDRGGYPFDPWGQGTLVTVSS

SEQ ID NO: 3 - protein, 123 aa, synthetic construct
  REGION 1..123: note = MF8224
QVQLMQSGSELKKPGASVKVSCKASGYTFTTYSMNWVRQAPGQGLEWMGWINTNTGNPTYAQDFTGRFVFSLDTSVSTAYLQISSLKAEDTAVYYCARETYYYDSSGYPFDPWGQGTLVTVSS

SEQ ID NO: 4 - protein, 123 aa, synthetic construct
  REGION 1..123: note = MF8239
QVQLVQSGSELKKPGASVKVSCKASGYTFTDYAMNWVRQVPGQGLEWMGWINTYTGNPTYVQGFTGRFVFSLDTSVSTAYLQISSLKAEDTAVYYCARETYYYDSSGFPFDPWGQGTLVTVSS

SEQ ID NO: 5 - protein, 123 aa, synthetic construct
  REGION 1..123: note = MF8242
QVQLVQSGSELKKPGASVKVSCKASGYTFTNYAMNWVRQAPGQGLEWMGWINTNTGNPTYAQGFTGRFVFPLDTSVSTTYLQISSLKAEDTAVYYCARETYYYQSSGYLFDPWGQGTLVTVSS

SEQ ID NO: 6 - protein, 123 aa, synthetic construct
  REGION 1..123: note = MF8237
QVQLVQSGSELKKPGASVKVSCKASGYTFTSFGMSWVRQAPGQGLEWMGWINTNTGNPTYAQGFTGRFVFSLDTSVSTAYLQINSLKAEDTAVYYCARESYYYDRNDYPFDPWGQGTLVTVSS

SEQ ID NO: 7 - protein, 123 aa, synthetic construct
  REGION 1..123: note = MF8240
QVQLVQSGSELKKPGASVKVSCKASGYTFTTYSMNWVRQAPGQGLEWMGWINTNTGNPTYAQGFTGRFVFSLDTSVSTAYLQISSLNTEDTAVYYCARETYYYDVGGYPFDPWGQGTLVTVSS

SEQ ID NO: 8 - protein, 123 aa, synthetic construct
  REGION 1..123: note = MF8234
QVQLVQSGSELEKPGASVKVSCKASGYTFISYAMNWVRQAPGQGLEWMGWINTYTGNPTYAQGFTGRFVFSLDTSVSTAYLQISSLKAEDTAVYYCARETYYYDSGGYPFDPWGQGTLVTVSS

SEQ ID NO: 9 - protein, 123 aa, synthetic construct
  REGION 1..123: note = MF8245
QVQLVQSGSELKKPGASVKVSCKASGYTFTSYAVNWVRQAPGQGLEWMGWINTYTGNPTYAQGFTGRFVFSSDTSVNTAYLQISSLKAEDTAVYYCARETYFYDSSGYPFDPWGQGTLVTVSS

SEQ ID NO: 10 - protein, 123 aa, synthetic construct
  REGION 1..123: note = MF8231
QVQVVQSGSEVKKPGASVKVSCKASGYTFTTYSMNWVRQAPGQGLEWMGWINTYTGDPTYVQGFTGRFVFSLDTSVSTAYLQISSLKAEDTAVYYCARETYFYDRGGYPFDPWGQGTLVTVSS

SEQ ID NO: 11 - protein, 123 aa, synthetic construct
  REGION 1..123: note = MF8247
QVQLVQSGSELKKPGASVKVSCKASGYTFTDYAMNWVRQAPGQGLEWMGWINTYTGNPTYVQGFTGRFVFSLDTSVSTAYLQISSLKAEDTAVYYCARETYYYDSSAYPFDPWGQGTLVTVSS

SEQ ID NO: 12 - protein, 123 aa, synthetic construct
  REGION 1..123: note = MF8238
QVQLVQSGSELEKPGASVKVSCKASGYTFTTYSMNWVRQAPGQGLEWMGWINTYTGSPTYAQGFTGRFVFSLDTSVSTAYLQISSLKAEDTAIYYCARETFYFDSGGYPFDPWGQGTLVTVSS

SEQ ID NO: 13 - protein, 123 aa, synthetic construct
  REGION 1..123: note = MF8230
QVQLVQSGSELKKPGASVKVSCKASGYTFTTYSMNWVRQAPGQGLEWMGWINTYTGDPTYAQGFTGRFVFSLDTSVSTAYLQISSLKAEDTAVYYCARETYFYDRGGYPFDPWGQGTLVTVSS

SEQ ID NO: 14 - protein, 123 aa, synthetic construct
  REGION 1..123: note = MF8248
QVQLVQSGSELKKPGASVKVSCKASGYTFTTYAINWVRQAPGQGLEWMGWINTNTGNPTYAQGFTGRFVFSLDTSVSTAHLQISSLKAEDTAVYYCARETYYYATSGYPFDPWGQGALVTVSS

SEQ ID NO: 15 - protein, 123 aa, synthetic construct
  REGION 1..123: note = MF8246
QVQLVQSGSELKKPGASVKVSCKASGYTFTTYAMNWVRQAPGQGLEWMGWINTYTGDPTYAQGFTGRFVFSLDTSVNTAYLQISSLKAEDTAVYYCARETSYYDRTGYPFDPWGQGTLVTVSS

SEQ ID NO: 16 - protein, 123 aa, synthetic construct
  REGION 1..123: note = MF8223
QVQLVQSGSELKKPGASVKVSCKASGYTFTTYAMNWVRQAPGQGLEWMGWINTNTGNPTYAQGFTGRFVFSLDTSDSTAFLQISSLKAEDTAVYYCARETYYYDSSGYPFDPWGQGTLVTVSS

SEQ ID NO: 17 - protein, 123 aa, synthetic construct
  REGION 1..123: note = MF8222
QVELVQSGSELKKPGASVKVSCKASGYTFTTYSMNWVRQAPGQGLEWMGWINTNTGTPTYAQGFTGRFVFSLDTSVSTAYLQISSLKAEDTAVYYCARETYYYDSSGYPFDPWGQGTLVTVSS

SEQ ID NO: 18 - protein, 123 aa, synthetic construct
  REGION 1..123: note = MF8235
QVQLVQSGSELKKPGASVKVSCKASGYTFTTYSMNWVRQAPGQGLEWMGWINTNTGTPTYAQGFTGRFVFSLDTSVSTAYLQISSLKAEDTAVYYCARETYYYGSSGYPFAPWGQGTLVTVSS

SEQ ID NO: 19 - protein, 123 aa, synthetic construct
  REGION 1..123: note = MF8236
QVQLVQSGSELKKPGASVKVSCKASGYTFTTYSMNWVRQAPGQGLEWMGWINTYTGNPTYAQGFTGRFVFSLDTSVSTAYLQISSLTTEDTAVYYCARETYYYESSGYPFDPWGQGTLVTVSS

SEQ ID NO: 20 - protein, 123 aa, synthetic construct
  REGION 1..123: note = MF8241
QVQLVQSGSELEKPGASVKVSCKASGYTFTTYSMNWVRQAPGQGLEWMGWINTYTGSPTYAQGFTGRFVFSLDTSVSTAYLQISSLKAEDTAVYYCARETYYFDSGDYPFDPWGQGTLVTVSS

SEQ ID NO: 21 - protein, 123 aa, synthetic construct
  REGION 1..123: note = MF8244
QVQLVQSGSELEKPGASVKVSCKASGYTFTTYSMNWVRQAPGQGLEWMGWINTYTGSPTYAQGFTGRFVFSLDTSVSTAYLQISSLKAEDTAVYYCARETYYFDSGGYPFDPWGQGTLVTVSS

SEQ ID NO: 22 - protein, 123 aa, synthetic construct
  REGION 1..123: note = MF8221
QVQLVQSGSELKKPGASVKVSCKASGYTFTDYAMNWVRQAPGQGLEWMGWINTYTGNPTYVQGFTGRFVFSLDTSVSTAYLQISSLKAEDTAVYYCARETYYYDSSGYPFDPWGQGTLVTVSS

SEQ ID NO: 23 - protein, 123 aa, synthetic construct
  REGION 1..123: note = MF4356
QVQLVQSGSELKKPGASVKVSCKASGYTFTSYSMNWVRQAPGQGLEWMGWINTYTGDPTYAQGFTGRYVFSLDTSVNTAYLQISSLKAEDTAVYYCARETYYYDRGGYPFDPWGQGTLVTVSS

SEQ ID NO: 24 - protein, 5 aa, synthetic construct
  REGION 1..5: note = CDR1
SYGIS

SEQ ID NO: 25 - protein, 17 aa, synthetic construct
  REGION 1..17: note = CDR2
  VARIANT 6: note = May also be S
  VARIANT 7: note = May also be G
WISAYNANTNYAQKLQG

SEQ ID NO: 26 - protein, 10 aa, synthetic construct
  REGION 1..10: note = CDR3
  VARIANT 1: note = May also be G
  VARIANT 2: note = May also be S or Y
  VARIANT 3: note = May also be L or Y
  VARIANT 4: note = May also be W
  VARIANT 10: note = May also be G
DRHDHWWLDA

SEQ ID NO: 27 - protein, 5 aa, synthetic construct
  REGION 1..5: note = preferred option for C-terminal part of CDR3
DRHWD

SEQ ID NO: 28 - protein, 5 aa, synthetic construct
  REGION 1..5: note = CDR1
SYSMN

SEQ ID NO: 29 - protein, 17 aa, synthetic construct
  REGION 1..17: note = CDR2
WINTYTGDPTYAQGFTG

SEQ ID NO: 30 - protein, 14 aa, synthetic construct
  REGION 1..14: note = CDR3
ETYYYDRGGYPFDP

SEQ ID NO: 31 - protein, 14 aa, synthetic construct
  REGION 1..14: note = CDR3
ETYFYDRGGYPFDP

SEQ ID NO: 32 - protein, 12 aa, synthetic construct
  REGION 1..12: note = CDR3 variant
  VARIANT 1: note = Can also be G
  VARIANT 2: note = Can also be S or Y
  VARIANT 3: note = Can also be L or Y
  VARIANT 4: note = Can also be W
  SITE 5: note = misc_feature - Xaa can be any naturally occurring amino acid
  VARIANT 10: note = Can also be G
DRHDXHWWLDAF

SEQ ID NO: 33 - protein, 13 aa, synthetic construct
  REGION 1..13: note = CDR3 variant
  VARIANT 1: note = Can also be G
  VARIANT 2: note = Can also be S or Y
  VARIANT 3: note = Can also be L or Y
  VARIANT 4: note = Can also be W
  SITE 5: note = misc_feature - Xaa can be any naturally occurring amino acid
  VARIANT 10: note = Can also be G
DRHDXHWWLDAFD

SEQ ID NO: 34 - protein, 14 aa, synthetic construct
  REGION 1..14: note = CDR3 variant
  VARIANT 1: note = Can also be G
  VARIANT 2: note = Can also be S or Y
  VARIANT 3: note = Can also be L or Y
  VARIANT 4: note = Can also be W
  SITE 5: note = misc_feature - Xaa can be any naturally occurring amino acid
  VARIANT 10: note = Can also be G
DRHDXHWWLDAFDY

SEQ ID NO: 35 - protein, 11 aa, synthetic construct
  REGION 1..11: note = CDR3
DRHWHWWLDAF

SEQ ID NO: 36 - protein, 12 aa, synthetic construct
  REGION 1..12: note = CDR3
DRHWHWWLDAFD

SEQ ID NO: 37 - protein, 13 aa, synthetic construct
  REGION 1..13: note = CDR3
DRHWHWWLDAFDY

SEQ ID NO: 38 - protein, 6 aa, synthetic construct
  REGION 1..6: note = CDR1-tail
QSISSY

SEQ ID NO: 39 - protein, 7 aa, synthetic construct
  REGION 1..7: note = CDR3-tail
QQSYSTP

SEQ ID NO: 40 - protein, 107 aa, synthetic construct
  REGION 1..107: note = light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 41 - protein, 108 aa, synthetic construct
  REGION 1..108: note = light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 42 - protein, 1210 aa, Homo sapiens [sequence omitted]

SEQ ID NO: 43 - protein, 378 aa, Homo sapiens
  SIGNAL 1..23
  DOMAIN 24..378: note = ECD
MRPSGTAGAALLALLAALCPASRALEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 44 - protein, 1210 aa, Macaca mulatta
  SIGNAL 1..23
  DOMAIN 24..641: note = ECD
MGPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSEFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDTLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSSQKTKIISNRGENSCKATGQVCHALCSPEGCWGPEPRDCVSCQNVSRGRECVDKCNILEGEPREFVENSECIQCHPECLPQVMNITCTGRGPDNCIQCAHYIDGPHCVKTCPAGVMGENNTLVWKYADAGHVCHLCHPNCTYGCTGPGLEGCARNGPKIPSIATGMLGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 45 - protein, 16 aa, synthetic construct
  REGION 1..16: note = CDR2
WINTYTGDPTYAQGFT

SEQ ID NO: 46 - protein, 1408 aa, Homo sapiens
  SIGNAL 1..24
  DOMAIN 25..950: note = ECD
  DOMAIN 951..973: note = TM
  DOMAIN 974..1408: note = intracellular tail
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 47 - protein, 16 aa, synthetic construct
  REGION 1..16: note = EFGR CDR3
EGYYETTTYYYNLFDS

SEQ ID NO: 48 - protein, 11 aa, synthetic construct
  REGION 1..11: note = EGFR CDR3
ERFLEWLHFDY

SEQ ID NO: 49 - protein, 10 aa, synthetic construct
  REGION 1..10: note = EGFR CDR3
DRNWGWDFDY

SEQ ID NO: 50 - protein, 13 aa, synthetic construct
  REGION 1..13: note = EGFR CDR3
DRHWHWWLDAFDY

SEQ ID NO: 51 - protein, 11 aa, synthetic construct
  REGION 1..11: note = EGFR CDR3
GDLFITGTLDY

SEQ ID NO: 52 - protein, 14 aa, synthetic construct
  REGION 1..14: note = c-MET CDR3
KLEPTGYYYYYMDV

SEQ ID NO: 53 - protein, 14 aa, synthetic construct
  REGION 1..14: note = c-MET CDR3
KTSRYSGYHYYMDV

SEQ ID NO: 54 - protein, 8 aa, synthetic construct
  REGION 1..8: note = c-MET CDR3
AHYDILTG

SEQ ID NO: 55 - protein, 12 aa, synthetic construct
  REGION 1..12: note = c-MET CDR3
GKSHYSWDAFDY

SEQ ID NO: 56 - protein, 20 aa, synthetic construct
  REGION 1..20: note = c-MET CDR3
GTYYYGSGSFSTRVFDAFDV

SEQ ID NO: 57 - protein, 14 aa, synthetic construct
  REGION 1..14: note = c-MET CDR3
QSRRYSGYASYFDY

SEQ ID NO: 58 - protein, 14 aa, synthetic construct
  REGION 1..14: note = c-MET CDR3
QRRAYSGYNWYFDL

SEQ ID NO: 59 - protein, 12 aa, synthetic construct
  REGION 1..12: note = c-MET CDR3
RNDFWSGYLFDY

SEQ ID NO: 60 - protein, 13 aa, synthetic construct
  REGION 1..13: note = c-MET CDR3
KTTVGYYYYYMDV

SEQ ID NO: 61 - protein, 13 aa, synthetic construct
  REGION 1..13: note = c-MET CDR3
GPELGYYYYYMDI

SEQ ID NO: 62 - protein, 17 aa, synthetic construct
  REGION 1..17: note = c-MET CDR3
ASSMITFGGVIVSWFDP

SEQ ID NO: 63 - protein, 14 aa, synthetic construct
  REGION 1..14: note = c-MET CDR3
RVNRYSGYATYFDL

SEQ ID NO: 64 - protein, 14 aa, synthetic construct
  REGION 1..14: note = c-MET CDR3
ETYYYDRGGYPFDP

SEQ ID NO: 65 - protein, 14 aa, synthetic construct
  REGION 1..14: note = c-MET CDR3
RTSRYSGYHYYLDV

SEQ ID NO: 66 - protein, 8 aa, synthetic construct
  REGION 1..8: note = c-MET CDR3
LLYDLFDL

SEQ ID NO: 67 - protein, 13 aa, synthetic construct
  REGION 1..13: note = c-MET CDR3
SIDMATITDAFDI

SEQ ID NO: 68 - protein, 15 aa, synthetic construct
  REGION 1..15: note = c-MET CDR3
GTTGNPYYFYYYMDV

SEQ ID NO: 69 - protein, 127 aa, synthetic construct
  REGION 1..127: note = heavy chain variable region
  DOMAIN 1..30: note = FR1
  DOMAIN 31..35: note = CDR1
  DOMAIN 36..49: note = FR2
  DOMAIN 50..66: note = CDR2
  DOMAIN 67..97: note = FR3
  DOMAIN 98..116: note = CDR3
  DOMAIN 117..127: note = FR4
EVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSS

SEQ ID NO: 70 - protein, 122 aa, synthetic construct
  REGION 1..122: note = heavy chain variable region
  DOMAIN 1..30: note = FR1
  DOMAIN 31..35: note = CR1
  DOMAIN 36..49: note = FR2
  DOMAIN 50..66: note = CDR2
  DOMAIN 67..97: note = FR3
  DOMAIN 98..111: note = CDR3
  DOMAIN 112..122: note = FR4
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAKDRHWHWWLDAFDYWGQGTLVTVSS

SEQ ID NO: 71 - protein, 120 aa, synthetic construct
  REGION 1..120: note = heavy chain variable region
  DOMAIN 1..30: note = FR1
  DOMAIN 31..35: note = CDR1
  DOMAIN 36..49: note = FR2
  DOMAIN 50..66: note = CDR2
  DOMAIN 67..97: note = FR3
  DOMAIN 98..109: note = CDR3
  DOMAIN 110..120: note = FR4
QVQLVQSGSELKKPGASVKISCKASGYDFTNYAMNWVRQAPGHGLEWMGWINANTGDPTYAQGFTGRFVFSLDTSVSTAYLQISSLKAEDSAVYYCTRERFLEWLHFDYWGQGTLVTVSS

SEQ ID NO: 72 - protein, 128 aa, synthetic construct
  REGION 1..128: note = heavy chain variable region
  DOMAIN 1..30: note = FR1
  DOMAIN 31..35: note = CDR1
  DOMAIN 36..49: note = FR2
  DOMAIN 50..67: note = CDR2
  DOMAIN 68..100: note = FR3
  DOMAIN 101..117: note = CDR3
  DOMAIN 118..128: note = FR4
EVQLVESGGGLVKPGGSLRLSCAASGFTFSKAWMNWVRQAPGKGLEWVGRIKSKTDGGTTDYAAPVKGRFTISRDDSKNTLYLQMNSLKTEDTAVYYCTTASSMITFGGVIVSWFDPWGQGTLVTVSS

SEQ ID NO: 73 - protein, 123 aa, synthetic construct; identical to SEQ ID NO: 23
  REGION 1..123: note = heavy chain variable region
  DOMAIN 1..30: note = FR1
  DOMAIN 31..35: note = CDR1
  DOMAIN 36..49: note = FR2
  DOMAIN 50..66: note = CDR2
  DOMAIN 67..97: note = FR3
  DOMAIN 98..112: note = CDR3
  DOMAIN 113..123: note = FR4

SEQ ID NO: 74 - protein, 125 aa, synthetic construct
  REGION 1..125: note = heavy chain variable region
  DOMAIN 1..30: note = FR1
  DOMAIN 31..35: note = CDR1
  DOMAIN 36..49: note = FR2
  DOMAIN 50..66: note = CDR2
  DOMAIN 67..97: note = FR3
  DOMAIN 98..114: note = CDR3
  DOMAIN 115..125: note = FR4
EVQLVESGGGVVQPGRSLRLSCAASGFTFSNYGMHWVRQAPGKGLEWVAVISYDGSTKYSADSLKGRFTISRDNSKNTLYLQMNSLRADDTAVYYCAKEGWSFDSSGYRSWFDSWGQGTLVTVSS

SEQ ID NO: 75 - protein, 122 aa, synthetic construct
  REGION 1..122: note = MF3353
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDSYWHWWLGAFDYWGQGTLVTVSS

SEQ ID NO: 76 - protein, 122 aa, synthetic construct
  REGION 1..122: note = MF8229
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCARDSYWHWWLGAFDYWGQGTLVTVSS

SEQ ID NO: 77 - protein, 122 aa, synthetic construct
  REGION 1..122: note = MF8228
QVQLVQSGAEVKKPGASVKVSCKASGYTFTSYGISWVRQAPGQGLEWMGWISAYSGNTNYAQKLQGRVTMTTDTSTSTAYMELRSLRSDDTAVYYCARDSYWHWWLGAFDYWGQGTLVTVSS

SEQ ID NO: 78 - protein, 122 aa, synthetic construct; identical to SEQ ID NO: 70
  REGION 1..122: note = MF3370

SEQ ID NO: 79 - protein, 122 aa, synthetic construct
  REGION 1..122: note = MF8233
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCAKDRHWHWWLDAFDYWGQGTLVTVSS

SEQ ID NO: 80 - protein, 122 aa, synthetic construct
  REGION 1..122: note = MF8232
QVQLVQSGAEVKKPGASVKVSCKASGYTFTSYGISWVRQAPGQGLEWMGWISAYSGNTNYAQKLQGRVTMTTDTSTSTAYMELRSLRSDDTAVYYCAKDRHWHWWLDAFDYWGQGTLVTVSS

SEQ ID NO: 81 - protein, 122 aa, synthetic construct
  REGION 1..122: note = MF3393
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGYLDHWWLGAFDYWGQGTLVTVSS

SEQ ID NO: 82 - protein, 122 aa, synthetic construct
  REGION 1..122: note = MF8227
QVQLVQSGAEVKKPGASVKVSCKASGYTFTSYGISWVRQAPGQGLEWMGWISAYNANTNYAQKLQGRVTMTTDTSTSTAYMELRSLRSDDTAVYYCARGYLDHWWLGAFDYWGQGTLVTVSS

SEQ ID NO: 83 - protein, 122 aa, synthetic construct
  REGION 1..122: note = MF8226
QVQLVQSGAEVKKPGASVKVSCKASGYTFTSYGISWVRQAPGQGLEWMGWISAYSGNTNYAQKLQGRVTMTTDTSTSTAYMELRSLRSDDTAVYYCARGYLDHWWLGAFDYWGQGTLVTVSS

SEQ ID NO: 84 - protein, 107 aa, synthetic construct; identical to SEQ ID NO: 40
  REGION 1..107: note = common light chain

SEQ ID NO: 85 - protein, 107 aa, synthetic construct [sequence omitted]

SEQ ID NO: 86 - DNA, 321 bp, synthetic construct
  misc_feature 1..321: note = common light chain
  source 1..321: mol_type = other DNA
  CDS 1..321
gacatccagatgacccagtctccatcctccctgtctgcatctgtaggagacagagtcaccatcacttgccgggcaagtcagagcattagcagctacttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccaacgttcggccaagggaccaaggtggagatcaaa

SEQ ID NO: 87 - protein, 107 aa, synthetic construct; identical to SEQ ID NO: 40
  REGION 1..107: note = Synthetic Construct

SEQ ID NO: 88 - DNA, 324 bp, synthetic construct
  misc_feature 1..324: note = common light chain
  source 1..324: mol_type = other DNA
  CDS 1..324
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 89 - protein, 107 aa, synthetic construct [sequence omitted]

SEQ ID NO: 90 - protein, 108 aa, synthetic construct; identical to SEQ ID NO: 41
  REGION 1..108: note = GKV1-39/jk5 common light chain

SEQ ID NO: 91 - protein, 95 aa, synthetic construct
  REGION 1..95: note = V-region IGKV1-39A
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 92 - DNA, 294 bp, synthetic construct
  misc_feature 1..294: note = CH1 region
  source 1..294: mol_type = other DNA
  CDS 1..294
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagtt

SEQ ID NO: 93 - protein, 98 aa, synthetic construct
  REGION 1..98: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 94 - DNA, 45 bp, synthetic construct
  misc_feature 1..45: note = hinge
  source 1..45: mol_type = other DNA
  CDS 1..45
gagcccaaatcttgtgacaaaactcacacatgcccaccgtgccca

SEQ ID NO: 95 - protein, 15 aa, synthetic construct
  REGION 1..15: note = Synthetic Construct
EPKSCDKTHTCPPCP

SEQ ID NO: 96 - DNA, 330 bp, synthetic construct
  misc_feature 1..330: note = CH2 region
  source 1..330: mol_type = other DNA
  CDS 1..330
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 97 - protein, 110 aa, synthetic construct
  REGION 1..110: note = Synthetic Construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 98 - DNA, 330 bp, synthetic construct
  misc_feature 1..330: note = CH2 containing L235G and G236R silencing substitutions:
  source 1..330: mol_type = other DNA
  CDS 1..330
gcacctgaactcggcaggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 99 - protein, 110 aa, synthetic construct
  REGION 1..110: note = Synthetic Construct
APELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 100 - DNA, 321 bp, synthetic construct
  misc_feature 1..321: note = CH3: KK of DEKK
  source 1..321: mol_type = other DNA
  CDS 1..321
gggcagccccgagaaccacaggtgtacaccaagcccccatcccgggaggagatgaccaagaaccaggtcagcctgaagtgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 101 - protein, 106 aa, synthetic construct
  REGION 1..106: note = Synthetic Construct
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - DNA, 321 bp, synthetic construct
  misc_feature 1..321: note = CH3: DE of DEKK
  source 1..321: mol_type = other DNA
  CDS 1..321
gggcagccccgagaaccacaggtgtacaccgaccccccatcccgggaggagatgaccaagaaccaggtcagcctgacctgcgaggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggttga

SEQ ID NO: 103 - protein, 106 aa, synthetic construct
  REGION 1..106: note = Synthetic Construct
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 30 aa, synthetic construct
  REGION 1..30: note = FR1
EVQLVETGAEVKKPGASVKVSCKASDYIFT

SEQ ID NO: 105 - protein, 5 aa, synthetic construct
  REGION 1..5: note = CDR1
KYDIN

SEQ ID NO: 106 - protein, 14 aa, synthetic construct
  REGION 1..14: note = FR2
WVRQAPGQGLEWMG

SEQ ID NO: 107 - protein, 17 aa, synthetic construct
  REGION 1..17: note = CDR2
WMSANTGNTGYAQKFQG

SEQ ID NO: 108 - protein, 32 aa, synthetic construct
  REGION 1..32: note = FR3
RVTMTRDTSINTAYMELSSLTSGDTAVYFCAR

SEQ ID NO: 109 - protein, 18 aa, synthetic construct
  REGION 1..18: note = CDR3
SSLFKTETAPYYHFALDV

SEQ ID NO: 110 - protein, 11 aa, synthetic construct
  REGION 1..11: note = FR4
WGQGTTVTVSS

SEQ ID NO: 111 - protein, 30 aa, synthetic construct
  REGION 1..30: note = FR1
QVQLVQSGAEVKKPGASVKVSCKASGYTFT

SEQ ID NO: 112 - protein, 14 aa, synthetic construct
  REGION 1..14: note = FR2
WVRQAPGQGLEWMG

SEQ ID NO: 113 - protein, 17 aa, synthetic construct
  REGION 1..17: note = CDR2
WISAYNGNTNYAQKLQG

SEQ ID NO: 114 - protein, 32 aa, synthetic construct
  REGION 1..32: note = FR3
RVTMTTDTSTSTAYMELRSLRSDDTAVYYCAK

SEQ ID NO: 115 - protein, 11 aa, synthetic construct
  REGION 1..11: note = FR4
WGQGTLVTVSS

SEQ ID NO: 116 - protein, 30 aa, synthetic construct
  REGION 1..30: note = FR1
QVQLVQSGSELKKPGASVKISCKASGYDFT

SEQ ID NO: 117 - protein, 5 aa, synthetic construct
  REGION 1..5: note = CDR1
NYAMN

SEQ ID NO: 118 - protein, 14 aa, synthetic construct
  REGION 1..14: note = FR2
WVRQAPGHGLEWMG

SEQ ID NO: 119 - protein, 17 aa, synthetic construct
  REGION 1..17: note = CDR2
WINANTGDPTYAQGFTG

SEQ ID NO: 120 - protein, 32 aa, synthetic construct
  REGION 1..32: note = FR3
RFVFSLDTSVSTAYLQISSLKAEDSAVYYCTR

SEQ ID NO: 121 - protein, 11 aa, synthetic construct
  REGION 1..11: note = FR4
WGQGTLVTVSS

SEQ ID NO: 122 - protein, 30 aa, synthetic construct
  REGION 1..30: note = FR1
EVQLVESGGGLVKPGGSLRLSCAASGFTFS

SEQ ID NO: 123 - protein, 5 aa, synthetic construct
  REGION 1..5: note = CDR1
KAWMN

SEQ ID NO: 124 - protein, 14 aa, synthetic construct
  REGION 1..14: note = FR2
WVRQAPGKGLEWVG

SEQ ID NO: 125 - protein, 19 aa, synthetic construct
  REGION 1..19: note = CDR2
RIKSKTDGGTTDYAAPVKG

SEQ ID NO: 126 - protein, 32 aa, synthetic construct
  REGION 1..32: note = FR3
RFTISRDDSKNTLYLQMNSLKTEDTAVYYCTT

SEQ ID NO: 127 - protein, 11 aa, synthetic construct
  REGION 1..11: note = FR4
WGQGTLVTVSS

SEQ ID NO: 128 - protein, 30 aa, synthetic construct
  REGION 1..30: note = FR1
QVQLVQSGSELKKPGASVKVSCKASGYTFT

SEQ ID NO: 129 - protein, 14 aa, synthetic construct
  REGION 1..14: note = FR2
WVRQAPGQGLEWMG

SEQ ID NO: 130 - protein, 32 aa, synthetic construct
  REGION 1..32: note = FR3
RYVFSLDTSVNTAYLQISSLKAEDTAVYYCAR

SEQ ID NO: 131 - protein, 11 aa, synthetic construct
  REGION 1..11: note = FR4
WGQGTLVTVSS

SEQ ID NO: 132 - protein, 30 aa, synthetic construct
  REGION 1..30: note = FR1
EVQLVESGGGVVQPGRSLRLSCAASGFTFS

SEQ ID NO: 133 - protein, 5 aa, synthetic construct
  REGION 1..5: note = CDR1
NYGMH

SEQ ID NO: 134 - protein, 14 aa, synthetic construct
  REGION 1..14: note = FR2
WVRQAPGKGLEWVA

SEQ ID NO: 135 - protein, 17 aa, synthetic construct
  REGION 1..17: note = CDR2
VISYDGSTKYSADSLKG

SEQ ID NO: 136 - protein, 32 aa, synthetic construct
  REGION 1..32: note = FR3
RFTISRDNSKNTLYLQMNSLRADDTAVYYCAK

SEQ ID NO: 137 - protein, 16 aa, synthetic construct
  REGION 1..16: note = CDR3
EGWSFDSSGYRSWFDS

SEQ ID NO: 138 - protein, 11 aa, synthetic construct
  REGION 1..11: note = FR4
WGQGTLVTVSS

SEQ ID NO: 139 - protein, 10 aa, synthetic construct
  REGION 1..10: note = CDR3
DRHWHWWLDA

SEQ ID NO: 140 - protein, 17 aa, synthetic construct
  REGION 1..17: note = CDR2
WISAYSGNTNYAQKLQG

SEQ ID NO: 141 - protein, 23 aa, synthetic construct
  REGION 1..23: note = signal peptide
MRPSGTAGAALLALLAALCPASR

SEQ ID NO: 142 - protein, 622 aa, synthetic construct
  REGION 1..622: note = ECD of human EGFR
ALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 143 - protein, 23 aa, synthetic construct
  REGION 1..23: note = predicted TM region
IATGMVGALLLLLVVALGIGLFM

SEQ ID NO: 144 - protein, 542 aa, synthetic construct
  REGION 1..542: note = intracellular tail
RRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 145 - protein, 23 aa, synthetic construct
  REGION 1..23: note = signal peptide
MRPSGTAGAALLALLAALCPASR

SEQ ID NO: 146 - protein, 355 aa, synthetic construct
  REGION 1..355: note = ECD of EGFRvarIII
ALEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 147 - protein, 23 aa, synthetic construct
  REGION 1..23: note = signal peptide
MGPSGTAGAALLALLAALCPASR

SEQ ID NO: 148 - protein, 621 aa, synthetic construct [sequence omitted]

SEQ ID NO: 149 - protein, 908 aa, synthetic construct
  REGION 1..908: note = ECD of human cMET
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFT

SEQ ID NO: 150 - protein, 23 aa, synthetic construct
  REGION 1..23: note = transmembrane region
GLIAGVVSISTALLLLLGFFLWL

SEQ ID NO: 151 - protein, 434 aa, synthetic construct
  REGION 1..434: note = intracellular region
KKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 152 - protein, 5 aa, synthetic construct
  REGION 1..5: note = CDR1
TYSMN

SEQ ID NO: 153 - protein, 10 aa, synthetic construct
  REGION 1..10: note = synthetic construct
DSYWHWWLGA

SEQ ID NO: 154 - protein, 10 aa, synthetic construct
  REGION 1..10: note = CDR3
GYLDHWWLGA

SEQ ID NO: 155 - protein, 17 aa, synthetic construct
  REGION 1..17: note = MF8233_CDR2
WISAYNANTNYAQKLQG

SEQ ID NO: 156 - protein, 13 aa, synthetic construct
  REGION 1..13: note = MF8233_CDR3
DRHWHWWLDAFDY

SEQ ID NO: 157 - protein, 17 aa, synthetic construct
  REGION 1..17: note = MF8230_CDR2
WINTYTGDPTYAQGFTG

SEQ ID NO: 158 - skipped

SEQ ID NO: 159 - protein, 13 aa, synthetic construct
  REGION 1..13: note = CDR3
GYLDHWWLGAFDY